(19) United States
(12) Patent Application Publication
(10) Pub. No.: US 2010/0028952 A1
(43) Pub. Date: Feb. 4, 2010

(54) FERMENTATION AND PURIFICATION OF ACTINOMADURA CHROMOPROTEIN AND RELATED SPECIES

(75) Inventors: Eugene Joseph Vidunas, Middletown, NY (US); Gow-Jen Tsai, Airmont, NY (US); Ping Cai, New City, NY (US); Justin Moran, Valley Cottage, NY (US); Pamela Sue Fink, New City, NY (US); Lourdes Jeanette Gordon, White Plains, NY (US)

Correspondence Address: WYETH LLC;PATENT LAW GROUP, 5 GIRALDA FARMS, MADISON, NJ, 07940, US

(73) Assignee: WYETH, Madison, NJ (US)

(21) Appl. No.: 12/305,428

(22) PCT Filed: Jun. 21, 2007
(86) PCT No.: PCT/US2007/014568

§ 371 Date: Sep. 29, 2009

Related U.S. Application Data
(60) Provisional application No. 60/815,697, filed on Jun. 21, 2006.

(51) Int. Cl. C12P 21/00; C12N 1/20; C07K 1/00

(52) U.S. Cl. 435/71.2; 435/252.6; 530/416

(57) ABSTRACT

The present invention provides methods for production and purification of active chromoproteins produced by Actinomadura sp. 21G792. The chromoproteins are useful for developing pharmaceutical compositions and treating diseases such as cancer or bacterial infections.

The present application relates to U.S. Provisional Patent Application No. 60/815,697, filed on Jun. 21, 2006, which is incorporated herein by reference in its entirety for all purposes.

### SEQUENCE LISTING

This application includes a Sequence Listing setting forth the nucleic acid and amino acid sequences discussed herein.

### FIELD OF THE INVENTION

The present invention relates to methods for production and purification of active chromoproteins produced by Actinomadura sp. 21G792. The chromoproteins are useful for developing pharmaceutical compositions and treating diseases such as cancer or bacterial infections.

### BACKGROUND OF THE INVENTION

Enediynes, a potent class of cytotoxic polyketides produced by members of the Actinomycetales, have been used to treat cancer. The typical mode of action of the enediyne drugs is through single- and double-strand DNA cleavage. DNA cleavage is induced by hydrogen abstraction from the deoxyribose sugar backbone by a diradical generated from a Bergman-type cycloaromatization of the enediyne ring. Two enediynes are currently approved for the clinical treatment of cancer: calicheamicin conjugated to a CD33 monoclonal antibody (Mylotarg®, USA) and poly(styrene-co-maleic acid)-conjugated neocarzinostatin (Japan).

Enediyne natural products can be divided into two sub-categories. The first sub-class is characterized by a bicyclo[7,3,0]dodecadiyne (i.e., nine-membered) enediyne core or its precursor, and the second sub-class is characterized by a bicylco[7,3,1]tridecadiyne (i.e., ten-membered) enediyne core. Examples of the nine-membered enediynes include neocarzinostatin, C-1027, kedarcidin, macromomycin, N1999A2 and maduropeptin. Examples of the ten-membered sub-class include calicheamicin, esperamicin, dynemicin and namenamicin. An additional characteristic that distinguishes the nine-membered from the ten-membered enediynes is that with the exception of N1999A2, all nine-membered enediynes are produced as enediyne-protein complexes, wherein the enediyne chromophore is attached to an inactive apoprotein by non-covalent binding. For this reason the nine-membered enediynes are often referred to as chromoproteins. It is believed that the apoprotein plays the critical role of stabilizing the labile nine-membered enediyne chromophore and providing the targeted delivery of the cytotoxic chromophore to the chromatin.

The amino acid sequences of several apoproteins have been determined by directly sequencing the apoprotein or by deducing the amino acid from a cloned DNA sequence. The apoproteins identified to date are small, acidic proteins (108-114 amino acids, aa), which are generated from a pre-apoprotein by the removal of a 32-34 aa amino-terminal leader peptide. The biosynthetic pathways for two chromoproteins (neocarzinostatin and C-1027) have been cloned and sequenced. In these cases, the gene encoding the apoprotein was clustered with the genes required for the biosynthesis of the associated chromophore.

The apoprotein component of the chromoprotein complex presents an attractive target for the directed alteration of drug properties. For example, if the apoprotein amino acid or nucleic acid sequence is discovered, the chromophore-binding motif of the apoprotein can be altered using established molecular biology techniques, such as site-directed mutagenesis, to create a rationally altered apoprotein that binds its natural chromophore more strongly or weakly. Moreover, such alterations to the apoprotein could lead to, for example, a chromoprotein having decreased toxicity, or a chromophore having increased potency or stability. Additionally, extensive manipulation of the apoprotein could lead to an apoprotein with greatly altered binding specificities and, thus, the ability to function as a targeted drug delivery vehicle for molecules very different from the enediyne chromophore. Similarly, the enediyne chromophore is a target for modification. One way to alter the chromophore is to manipulate the genes involved in its biosynthesis.

Accordingly, there exists a need for novel chromoproteins, and for isolation and characterization of the genes and proteins involved in their synthesis. Further, methods for efficient production and purification of the chromoproteins is desirable.

### SUMMARY OF THE INVENTION

The present invention relates to the highly potent anti-cancer chromoprotein produced by a terrestrial actinomycete, Actinomadura sp. 21G792 (NRRL 30778). The Actinomadura sp. 21G792 chromoprotein is a non-covalent complex of an apoprotein and a chromophore comprising a nine-membered enediyne. The invention provides novel isoforms of the Actinomadura sp. 21G792 chromophore as well as chromoproteins comprising the novel isoforms. In particular, the invention provides active chromoproteins comprising chromophores having the structures:

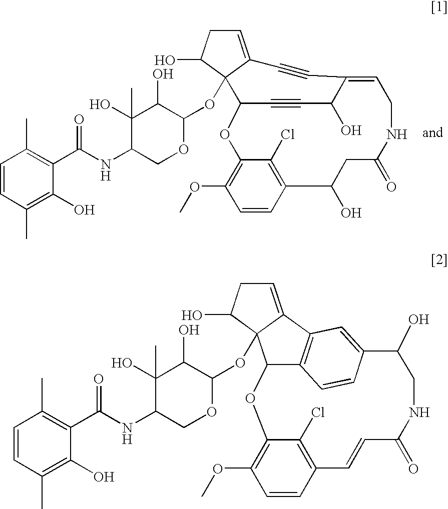

Structures [1] and [2] are otherwise referred to herein as chromophore-b and chromophore-c respectively.

In another aspect, the invention provides fermentation processes that result in improved chromoprotein yields. The fermentation process incorporates media that is formulated to maximize chromoprotein production and minimize chromoprotein instability and degradation.

In another aspect, the invention provides methods for purifying the Actinomadura sp. 21G792 chromoprotein. Scalable purification methods suitable for obtaining the chromoprotein from large scale fermentations are disclosed. Also disclosed are methods for separation of the chromoprotein isoforms.

The invention provides substantially pure forms of the Actinomadura sp. 21G792 chromoprotein and apoprotein, as well as pharmaceutical compositions comprising the chromoprotein and methods for administering the chromoprotein. Certain isoforms of the chromoprotein are shown to be useful for treatment of cancerous cells and tumors.

The present invention further provides a method for generating and purifying variants of the Actinomadura sp. 21G792 chromoprotein (including the apoprotein and chromophore species) that have altered biological activity. Such variant apoproteins can have altered chromophore binding properties, altered target specificity, or a combination thereof.

It will be understood that the present invention provides for production of large quantities of the apoprotein and the chromoprotein. It further will be appreciated that the invention may lead to the identification of other organisms capable of producing enediyne-related compounds or the identification of the genes involved in the synthesis of chromoproteins in, for example, organisms capable of producing enediyne related compounds, such as Actinomadura sp. 21G792. Additionally, it will be appreciated that the invention provides for the production of modified versions of the apoprotein which, for example, have decreased toxicity, increased potency, or increased stability. It also will be understood that manipulation of the Actinomadura sp. 21G792 apoprotein can lead to an apoprotein with altered binding specificities and, thus, the ability to function as a targeted drug delivery vehicle for chromophores different from the 21G792 enediyne chromophore. Finally, it will be appreciated that pharmaceutical compositions comprising the Actinomadura sp. 21G792 chromoprotein can be developed and administered to mammals, preferably humans, having bacterial infections or cancerous growths.

### BRIEF DESCRIPTION OF THE DRAWINGS

FIG. 1 is an HPLC chromatogram of the Actinomadura sp. 21G792 chromoprotein. The analytical conditions of the HPLC were as follows. Column: TosoHaas DEAE 5 PW (10 um particle size, 7.5 mm×7.5 cm in size). Buffer: 0-0.5 M linear gradient NaCl with constant 0.05 M Tris-HCl in 25 min at a flow rate of 0.8 ml/min.

FIG. 2 is a UV spectrum of the Actinomadura sp. 21G792 chromoprotein.

FIG. 3 is an HPLC chromatogram of the 21G792 apoprotein. The analytical conditions of the HPLC were as follows. Column: VYDAC Protein C4 (300 A, 3.0×100 mm in size). Solvent: 10-30% Acetonitrile in H₂O with constant 0.05% TFA in 6 minutes at 2 ml/min.

FIG. 4 is a UV spectrum of the 21G792 apoprotein.

FIG. 5 shows a molecular weight determination for the apoprotein (12.92409 kDa by MALDI-MS).

FIG. 6 depicts the structure of Actinomadura sp. 21G792 chromophore species (chromophores-b, -c, and -d).

FIG. 7 provides the nucleotide sequence and deduced amino acid sequence of the 21G792 pre-apoprotein and apoprotein. The putative ribosome binding site is boxed, and the leader peptide is underlined. The slash mark indicates the cleavage site for leader peptide and apoprotein.

FIG. 8 depicts the open reading frames of Actinomadura sp. 21G792 chromoprotein gene cluster. Genes located on cosmid 41417 are indicated by a solid line above the orf arrows. Those located on cosmid 21gD are indicated by the dashed line composed of small dashes, and those located on cosmid 21gB are indicated by the dashed line composed of large dashes. Locations of probes used to identify each cosmid are indicated by black barbells. PstI (P) and EcoRI (E) restriction sites are labeled.

FIG. 9 depicts a pathway for synthesis of the tyrosine-derived component (3-[2-chloro-3-hydroxy-4-methoxy-phenyl]-3-hydroxy-propionic acid) of the Actinomadura sp. 21G792 chromophore.

FIG. 10 depicts structural domains of the orf17 gene product. Core motifs of the condensation (C), adenylation (A) and peptidyl-carrier protein (PCP) domains are boxed and labeled. Residues contributing to the A domain substrate specificity code for the orf17 gene product and SgcC4 of the C-1027 biosythetic pathway are in bold and underlined. Identical residues are marked with an asterisk, a colon indicates conserved residues and a semi-colon indicates semi-conserved residues.

FIG. 11 depicts a pathway for synthesis of the madurosamine (4-amino-4-deoxy-3-C-methyl-β-ribopyranose) component of the Actinomadura sp. 21G792 chromophore.

FIG. 12 depicts the alignment of Orf38 with dNDP-glucose-4,6-dehydratases and UDP-glucuronate decarboxylases. Glucose-4,6-dehydratase sequences included in the alignment are Orf5 from the Streptomyces neyagawaensis concanamycin A gene cluster (AAZ94396), MtmE from the Streptomyces argillaceus mithramycin gene cluster (CAA71847), and SpcE from the Streptomyces spectabilis spectinomycin gene cluster (AAD31797). Glucuronate decarboxylase sequences included in the alignment are Uxs1 from Pisum sativum (BAB40967), Uxs3 from Arabidopsis thaliana (AAK70882), Uxs1 from Arabidopsis thaliena (AAK70880), Uxs2 from Arabidopsis thaliana (AAK70881), Uxs1 from Mus musculus (AAK85410) and Uxs1 from Cryptococcus neoformans (MK59981). Identical residues are marked with an asterisk, a colon indicates conserved residues and a semi-colon indicates semi-conserved residues.

FIG. 13 depicts a pathway for synthesis of the 2-hydroxy-3,6-dimethyl benzoic acid component of the Actinomadura sp. 21G792 chromophore.

FIG. 14 depicts the alignment of the region between the A4 and A5 core motifs of Orf31 and ten aryl acid-AMP ligases. Structural anchors are shaded in black. Proposed constituents of the carboxy acid binding pockets are shaded in grey. Residues proposed to be involved in discrimination between the activation of DHBA and salicylic acid are identified with a number sign. Identical residues are marked with an asterisk, a colon indicates conserved residues and a semi-colon indicates semi-conserved residues.

FIG. 15 depicts a biosynthetic pathway for the generation of the enediyne core of the Actinomadura sp. 21G792 chromophore.

FIG. 16 depicts the domain organization and comparison of Orf5 with the SgcE and NcsE enediyne PKSs. aa, amino acid; KS, ketosynthase; AT, acetyltransferase; ACP, acyl carrier protein; KR, ketoreductase; DH, dehydratase; TD, terminal domain.

FIG. 17 depicts a route to assembly of the four components of the Actinomadura sp. 21G792 chromophore.

FIG. 18 is a chromatogram showing an elution profile for the Actinomadura sp. 21G792 chromoprotein from a DEAE Sepharose anion exchange column.

FIG. 19 is a chromatogram showing an elution profile for the Actinomadura sp. 21G792 chromoprotein from a Phenyl Sepharose HP column.

FIG. 20 is a chromatogram showing an elution profile for the Actinomadura sp. 21G792 chromoprotein from a Superdex 75 size fractionation column.

FIG. 21 shows a Phenyl 5PW chromatogram (absorbance at 230 nm) of Superdex 75 fraction 6 (panel A) and absorbance spectra (200-400 nm) for the chromoprotein and apoprotein peaks (panel B).

FIG. 22 shows a BioSil SEC 125 chromatogram of Superdex 75 fraction 6 (panel A) and an absorbance spectrum for the coeluted chromoprotein and apoprotein (panel B).

FIG. 23 shows an SDS-PAGE analysis of chromoprotein-containing samples from broth and relevant column fractions from various steps of purification. Proteins were separated under reducing conditions and stained (Coomassie). The predominant band migrating between 31 and 36.5 kDa is identified as chromoprotein. Lanes 1 and 18: MW standards; Lane 2: clarified broth (DEAE load); Lane 3: DEAE 0.25 M pool; Lane 4: DEAE fraction D1; Lane 5: DEAE fraction D2; Lane 6: DEAE fraction D3; Lane 7: phenyl sepharose pool 8; Lane 8: phenyl sepharose fraction P9 (chromoprotein); Lane 9: phenyl sepharose fraction P10 (chromoprotein); Lane 10: phenyl sepharose fraction P11; Lane 11: phenyl sepharose fraction P14 (apoprotein); Lane 12: phenyl sepharose fraction P15 (apoprotein); Lane 13: Superdex 75 load (2 ul); Lane 14: Superdex fraction S5; Lane 15: Superdex fraction S6; Lane 16: Superdex fraction S7; Lane 17: Superdex fraction S8.

FIG. 24 shows an SDS-PAGE analysis of the Actinomadura sp. 21G792 chromoprotein (fraction S6) sequentially purified by anion exchange chromatography, hydrophobic interaction chromatography, and size exclusion chromatography. The predominant band migrating between 37.0 and 28.9 kDa is identified as chromoprotein. Lanes 1 and 5: MW standards; Lane 2: chromoprotein reference standard; Lane 3: Superdex 75 fraction S6; Lane 4: Mixture of reference standard and Superdex 75 fraction S6.

FIG. 25 shows separation of chromoproteins containing related chromophore isoforms. A chromoprotein preparation was separated using Phenyl Sepharose HP. Peak B corresponds to chromoprotein-b which comprises chromophore-b. Peak A includes chromoproteins-c and -d, comprising chromophores-c and -d. Apoprotein elutes separately from the chromoprotein species.

FIG. 26 shows the enediyene composition of the Phenyl Sepharose pools (Peaks A and B of FIG. 25).

FIG. 27 is a graph demonstrating that the 21G792 chromoprotein induced dose-dependent DNA strand breaks occur in p21-proficient and p21-deficient HCT116 human colon carcinoma cells at >100 ng/ml chromoprotein concentrations.

FIG. 28 is a DNA cleavage assay showing that the 21G792 chromoprotein induced single strand breaks and double strand breaks, the reaction continued to progress over 24 hours, and DNA cleavage did not require a thiol agent.

FIG. 29 depicts digestion of Histone H1 by the Actinomadura sp. 21G792 chromoprotein and inhibition by DNA. Protease inhibitors are PMSF, Leupeptin, Aprotinin, and Pepstatin A. The apoprotein has no activity.

FIG. 30 depicts relative sensitivity of histones H1, H2A, H2B, H3, and H4 to digestion by the Actinomadura sp. 21G792 chromoprotein. Basic proteins such as myelin basic protein, but not neutral/acidic proteins, are also susceptible to cleavage.

FIG. 31 depicts histone H1 reduction in cells treated with the Actinomadura sp. 21G792 chromoprotein, but not bleomycin or calicheamicin.

FIG. 32A is a protein immunoblot showing that exposure of HCT116 cells to the chromoprotein at various concentrations results in the activation of the p53/p21 checkpoint. FIG. 32B depicts phosphorylation of the serine-15 amino acid residue of p53 at the cleavage of poly-ADP-ribose phosphorylase (ParP).

FIGS. 33 and 34 are a series of graphs showing the in vivo potency of the 21G792 chromoprotein against tumors of subcutaneously injected LoVo (colon cancer); HCT116 (colon); HT29 (colon); LOX (melanoma); HN5 (head & neck); and PC-3 (prostate) cells in athymic (nude) mice.

FIG. 35 depicts uptake of FITC labeled Actinomadura sp. 21G792 chromoprotein by HCT116 cells.

FIG. 36 depicts uptake of FITC labeled Actinomadura sp. 21G792 chromoprotein and apoprotein by HCT116 cells.

FIG. 37 depicts uptake of labeled Actinomadura sp. 21G792 chromoprotein in the presence of a 10 fold greater concentration of unlabeled chromoprotein.

FIG. 38 depicts the effect of an energy uncoupling agent (sodium azide) or a tubulin disrupting agent (nocodazole) on uptake of the Actinomadura sp. 21G792 apoprotein by HCT116 cells.

FIG. 39 depicts linkage of a monoclonal antibody to a derivative of the Actinomadura sp. 21G792 chromophore.

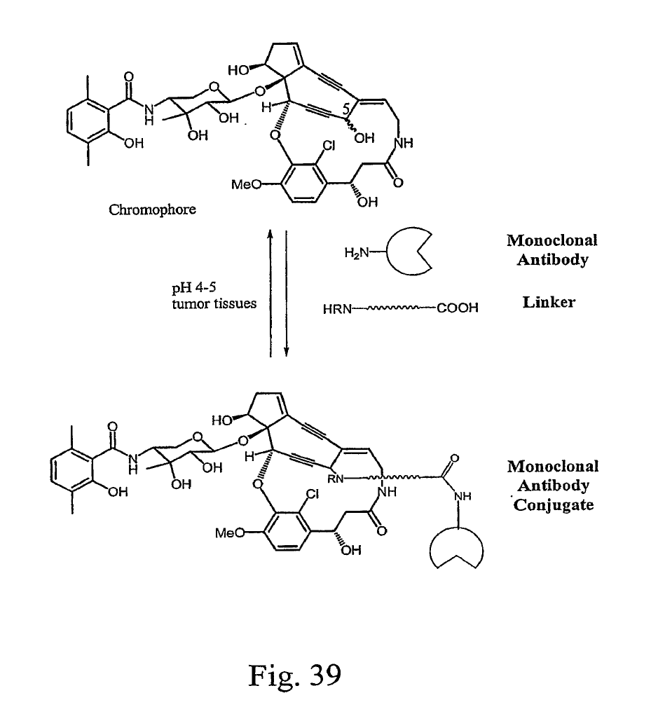

### DETAILED DESCRIPTION OF THE INVENTION

Enediyne antibiotics are produced by a variety of organisms generally belonging to the order Actinomycetales, including but not limited to the genera Streptomyces, Micromonospora, and Actinomadura. The present invention relates to a novel chromoprotein produced by Actinomadura sp. 21G792, deposited at the Agricultural Research Service Culture Collection (NRRL, 1815 North University Street, Peoria, Ill., 61064). The deposits were made under the terms of the Budapest Treaty. Actinomadura sp. 21G792 has been given accession number NRRL 30778. Of such organisms known to date, Actinomadura sp. 21G792 appears to be most similar to the Actinomadura strain deposited as ATCC 39144 (U.S. Pat. No. 4,546,084). As assessed by 16S rDNA sequences, the strains are related species or subspecies.

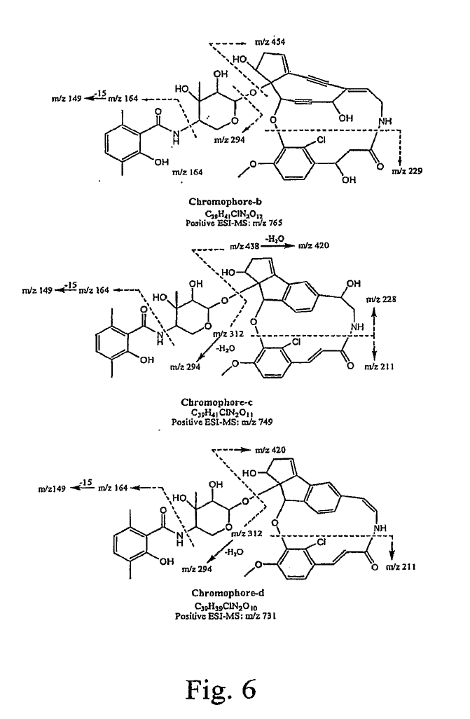

The present invention provides novel chromoproteins and chromophores. It has been discovered that Actinomadura sp. 21G792 produces multiple related chromophores. The chromophores are individually complexed with an apoprotein to form multiple related chromoproteins. Accordingly, the invention provides chromophore-b, chromophore-c, and chromophore-d, as depicted in FIG. 6. At least chromophore-b and chromophore-c have anti-tumor and anti-bacterial activities.

The invention provides methods for fermenting and cultivating Actinomadura sp. 21G792. Cultivation of Actinomadura sp. 21G792 may be carried out in a wide variety of liquid culture media. Media that are useful for the production of the Actinomadura sp. 21G792 chromoprotein include an assimilable source of carbon, such as dextrin, sucrose, molasses, glycerol, etc.; an assimilable source of nitrogen, such as protein, protein hydrolysate, polypeptides, amino acids, corn steep liquor, etc.; and inorganic anions and cations, such as potassium, sodium, iron, magnesium, ammonium, calcium, sulfate, carbonate, phosphate, chloride, etc. Trace elements such as boron, molybdenum, copper, etc., are often supplied as impurities of other constituents of the media. For example, a common nitrogen source Martone J-1 contains iron, magnesium, and other trace metals.

It has been discovered that inclusion of certain halide salts results in improved growth and a significant increase in chromoprotein production. Previously, the beneficial effect of including an iodide salt has been observed for production of an iodine-containing chromophore, but the effect of halides on production of the Actinomadura sp. 21G792 chromophore, which contains no iodine, was unexpected. Inclusion of a bromide salt also resulted in improved growth and chromoprotein yield. However, variation in the amount of chloride ion had no apparent effect.

Cultures of Actinomadura sp. 21G792 can use complex carbohydrates that contain glucose, such as molasses and hydrolyzed starch. For large scale fermentation, in order to achieve consistent results, it is desirable to use media comprising defined carbon sources. In addition to glucose, useful carbon sources that support growth and chromoprotein production in tube fermentation also include sucrose and maltrin. Carbon sources that do not support good chromoprotein yields include maltose, lactose, galactose, mannose, mannitol, glycerol, soybean oil, and cottonseed oil. In large scale fermentation, glucose is a preferred carbon source. In a large scale fermentation that included glucose, sucrose, and fructose, only glucose was utilized during the course of the fermentation (even though sucrose is assimilable in tube fermentation).

A useful nitrogen source for Actinomadura sp. 21G792 is peptone. However, non-animal derived nitrogen sources are generally desirable for production of pharmaceutical agents. Various non-animal nitrogen sources were tested, of which Marcor Martone J-1 provided particularly good results (see Examples). Other preferred non-animal nitrogen sources that produce comparable yields include Marcor Martone L-1, Marcor Bean Peptone, Amberferm 4415, Hy Soy T. Other examples of non-animal nitrogen sources that are useful but may produce lower yields include Amberferm 4000, Amberferm 4015, Amisoy, corn hydrolysate, wheat hydrolysate (DMV International #WGE80M), and Pharmamedia. No product was detected when the nitrogen source was soy hydrolysate (DMV International #SE50MAF) or a mixture of 75% soy hydrolysate and 25% wheat hydrolysate.

To obtain bioactive protein-containing products, it is important to avoid stresses that degrade proteins that have been secreted or released into the fermentation medium. In large scale fermentations, which generally involve mechanical agitation or mixing and introduction of air (or oxygen) through a sparger, careful selection of antifoam agents is necessary to provide good yields. According to the invention, Pluronic L-61, Pluracol P2000 (polypropylene glycols) and Antarox 17-R2 surfactants have been identified to be useful in the production of complexes of apoproteins and bioactive molecules. The surfactants are particularly useful for enediyne-containing chromoproteins such as the Actinomadura sp. 21G792 chromoprotein, kedarcidin, and C-1027. Among tested surfactants, Pluronic L-61 resulted in the highest yield of the Actinomadura sp. 21G792 chromoprotein. The chromoprotein was also produced with Pluracol P2000 (polypropylene glycols) and Antarox 17-R2, but in reduced amounts. Surfactants that yielded almost no chromoprotein were Pluronic L-31, Pluronic L-81, Pluronic L-101, Clerol FBA 515B, Clerol FBA 265, Clerol FBA 975-US, Clerol 5074, and Antarox BL-214.

In a particular embodiment of the invention, the fermentation medium comprises 8.75 g/L glucose monohydrate, 0.01 g/L ferrous sulfate heptahydrate, 0.02 g/L magnesium sulfate heptahydrate, 2.0 g/L calcium carbonate (Mississippi™ Lime), 4.0 g/L Marcor martone J-1, 2 g/L sodium acetate trihydrate, 0.5 g/L potassium iodide, and 0.5 g/L Pluronic L-61.

In another embodiment of the invention, the fermentation medium comprises 8.75 g/L glucose monohydrate, 0.01 g/L ferrous sulfate heptahydrate, 0.02 g/L magnesium sulfate heptahydrate, 2.0 g/L calcium carbonate (Mississippi™ Lime), 4.0 g/L Marcor martone J-1, 2 g/L sodium acetate trihydrate, 0.5 g/L sodium bromide, and 0.5 g/L Pluronic L-61.

The present invention also provides substantially pure proteins and polypeptides. The term “substantially pure” as used herein in reference to a given polypeptide means that the polypeptide is substantially free from other biological macromolecules. For example, the substantially pure polypeptide is at least about 75%, at least about 80%, at least about 85%, at least about 95%, or about 99% pure by dry weight. Purity can be measured by any appropriate standard method known in the art, for example, by column chromatography, polyacrylamide gel electrophoresis, or HPLC analysis. It will be appreciated that substantially pure proteins include substantially pure chromoproteins, which are complexes of an apoprotein and an enediyne chromophore.

In an embodiment of the invention, a chromoprotein is purified from fermentation medium or an extract of Actinomadura sp. 21G792 by anion exchange chromotography. The method provides for efficient concentration of the chromoprotein and removes (among other impurities) most of the undesired pigments produced in fermentation. In one embodiment, the anion exchange resin is DEAE Sepharose FF. A chromatography load containing the chromoprotein is adjusted to pH 8.3 and applied to the anion exchange resin. The chromoprotein species are eluted by increasing the ionic strength of the mobile phase. In a particular embodiment in which the chromoprotein is eluted with a 0.25 M NaCl step gradient, and most of the active material is recovered in the first two column volumes. Depending on the load volume and the column size, when purifying chromoprotein from clarified fermentation broth, the concentration can be more than 10 fold.

In another embodiment of the invention, the Actinomadura sp. 21G792 chromoprotein is purified by hydrophobic interaction chromatography. The purification method separates the chromoprotein from the apoprotein, removes other protein and non-protein components, and allows efficient concentration of the chromoprotein. This solid support may be any organic, inorganic or composite material, porous, super-porous or nonporous, which is adequate for chromatography that is derivatized, for example, with poly(alkene glycols) (poly(propylene glycol), poly(ethylene glycol)), alkanes, alkenes, alkynes, aryls, 1,4-butanediol diglycidyl ether or other molecules which confer a hydrophobic character to the support. In a particular example, Phenyl Sepharose HP is used at about pH 8.2 and the chromoprotein is eluted with a gradient to 100 mM NaCl.

Fractionations that rely on binding of chromoprotein to a matrix (e.g., anion exchange, hydrophobic interaction) is often performed using a column, but alternatively can be performed in a batch process. For example, as an alternative to ion exchange column chromatography, a batch process binding of the chromoprotein species to a suitable anion exchanger such as Sephadex A50 can be used. In a batch process, a chromoprotein-containing solution (e.g., clarified fermentation broth buffered by the addition 1/50th volume of 1M Tris pH 8.3 for storage) is batch absorbed overnight stirring at room temperature to Sephadex A50 slurry equilibrated in 20 mM Tris pH 8.3. Where the solution is clarified fermentation broth, a useful volume ratio of Sephadex A50 to clarified fermentation broth is 1:15. Upon determination of complete binding, the Sephadex is by harvested by filtration and washed, for example, with 12 volumes of 20 mM Tris pH 8.3. The chromoprotein species are eluted in batch mode with 20 mM Tris, 250 mM NaCl pH 8.3 after 30 min stirring. The filtrate is then adjusted to binding conditions suitable for the next purification step. For example, where the next step is binding to Phenyl Sepharose HP, as exemplified herein, binding conditions for Phenyl Sephaorese HP can be obtained by addition of solid ammonium sulfate to 1.5 M concentration and 0.45 μm filtration.

In a further embodiment of the invention, the Actinomadura sp. 21G792 chromoprotein is purified by size exclusion chromatography which removes high and low molecular weight protein components. For example, gel filtration chromatography uses a size exclusion resin to separate species by molecular weight. Other means include, but are not limited to, ultrafiltration with suitable exclusion membranes and hydrophobic techniques. Suitable matrixes comprise Sephacryl S-200, Superose 12, Sepharose S-300, Superdex, Trisacryl, acrylamide, Sephadex or similar resins known to those of skill in the art which are capable of fractionating the sample in the desired size range. Gel filtration may be carried out at a temperature of about 2° to about 25° C. Typically, chromatography steps for chromoprotein purification are carried out at room temperature. A suitable buffer comprises about 0.1 to about 1.0M salt, preferably NaCl, at a pH of about 7 to about 8.5. In one embodiment the separation range of the chromatography matrix has a lower bound of about 3×10³. In another embodiment, the separation range of the chromatography matrix has an upper bound of about 10⁶. In a particular embodiment of the invention, the size exclusion matrix is Superdex 75 which has a separation range from about 3×10³ to about 7×10⁵. When the separation matrix is Superdex 75, the chromoprotein preparation can be separated using 20 mM Tris, 100 mM NaCl pH 8.2. Those of skill in the art will be able to optimize the buffer used with a particular matrix. Collected chromoprotein-containing fractions can be pooled as desired, for example to maximize yield or to minimize impurities.

In preferred embodiments, two or more of the purification methods are employed sequentially. Concentration of the chromoprotein can be achieved in each of the purification steps, depending on the column volume (CV) and the number of eluted CVs that are pooled. Alternatively or additionally, the chromoprotein can be concentrated by other methods known in the art, such as ammonium sulfate precipitation, microconcentration, and the like.

According to the invention, one or more chromoprotein species can be isolated or purified. In an embodiment of the invention, Actinomadura sp. 21G792 is grown using fermentation conditions selected for chromoprotein yield. Typically, the chromoprotein species are purified by a three step process as outlined above. The chromoprotein species are separable by hydrophobic interaction chromatography. In the example provided, phenyl sepharose chromatography yielded two chromoprotein peaks (containing three chromoprotein species). Further, the relative amount of apoprotein was reduced.

Components of the chromoprotein and of the chromophore biosynthetic pathway, or precursors of those components (i.e., the pre-apoprotein), are encoded by a contiguous set of open reading frames (orfs) referred to as the chromoprotein biosynthetic gene cluster. Accordingly, the invention provides an isolated nucleic acid that encodes an orf of the Actinomadura sp. 21G792 chromoprotein biosynthetic gene cluster (See Table 1), or an expressed (i.e., processed) fragment thereof (e.g., an apoprotein; SEQ ID NO:150). In one embodiment, the invention provides a nucleic acid having a nucleotide sequence that encodes the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150. In a preferred embodiment, the nucleic acids comprise the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149. It will be appreciated that the nucleic acids of the invention include complementary sequences.

TABLE 1
Open Reading Frames of the 21G792 Chromoprotein Gene Cluster

| | | SEQ | | SEQ |
|---|---|---|---|---|
| | Start/Stop | ID | Length | ID |
| Orf | (bp) | NO | (aa) | NO |
| 9* | Start/1391 | 1 | incomplete | 2 |
| 8* | 1475/1861 | 3 | 128 | 4 |
| 7* | 1916/2371 | 5 | 151 | 6 |
| 6* | 2672/4270 | 7 | 532 | 8 |
| 5* | 4984/4349 | 9 | 211 | 10 |
| 4* | 5054/6631 | 11 | 525 | 12 |
| 3* | 6685/6891 | 13 | 68 | 14 |
| 2* | 7472/6984 | 15 | 162 | 16 |
| 1* | 8971/7475 | 17 | 498 | 18 |
| 1 | 9268/10263 | 19 | 331 | 20 |
| 2 | 10592/11494 | 21 | 300 | 22 |
| 3 | 11498/13534 | 23 | 678 | 24 |
| 4 | 13541/14533 | 25 | 330 | 26 |
| 5 | 14530/20364 | 27 | 1944 | 28 |
| 6 | 20369/20827 | 29 | 152 | 30 |
| 7 | 20824/21375 | 31 | 183 | 32 |
| 8 | 21372/22766 | 33 | 464 | 34 |
| 9 | 23607/22852 | 35 | 251 | 36 |
| 10 | 24877/23867 | 37 | 336 | 38 |
| 11 | 25277/25933 | 39 | 218 | 40 |
| 12 | 25930/27588 | 41 | 552 | 42 |
| 13 | 27602/28699 | 43 | 365 | 44 |
| 14 | 28792/29577 | 45 | 261 | 46 |
| 15 | 29591/30280 | 47 | 229 | 48 |
| 16 | 30631/30344 | 49 | 95 | 50 |
| 17 | 30845/34207 | 51 | 1120 | 52 |
| 18 | 34204/35817 | 53 | 537 | 54 |
| 19 | 35852/37498 | 55 | 548 | 56 |
| 20 | 37516/38898 | 57 | 460 | 58 |
| 21 | 39250/40578 | 59 | 442 | 60 |
| 22 | 40705/42282 | 61 | 525 | 62 |
| 23 | 43151/42654 | 63 | 165 | 64 |
| 24 | 43376/44761 | 65 | 461 | 66 |
| 25 | 44805/46031 | 67 | 408 | 68 |
| 26 | 46045/47190 | 69 | 381 | 70 |
| 27 | 47187/48416 | 71 | 409 | 72 |
| 28 | 49128/48430 | 73 | 232 | 74 |
| 29 | 49328/50728 | 75 | 466 | 76 |
| 30 | 50725/51582 | 77 | 285 | 78 |
| 31 | 53282/51636 | 79 | 548 | 80 |
| 32 | 58519/53279 | 81 | 1746 | 82 |
| 33 | 59639/58593 | 83 | 348 | 84 |
| 34 | 59897/61078 | 85 | 393 | 86 |
| 35 | 61119/61565 | 87 | 148 | 88 |
| 36 | 61568/62773 | 89 | 401 | 90 |
| 37 | 62785/64128 | 91 | 447 | 92 |
| 38 | 64131/65117 | 93 | 328 | 94 |
| 39 | 65134/66753 | 95 | 539 | 96 |
| 40 | 68054/66834 | 97 | 406 | 98 |
| 41 | 68270/69292 | 99 | 340 | 100 |
| 42 | 69375/70757 | 101 | 460 | 102 |
| 43 | 71889/70846 | 103 | 347 | 104 |
| 44 | 72452/72036 | 105 | 138 | 106 |
| 45 | 72706/74379 | 107 | 557 | 108 |
| 46 | 75114/74422 | 109 | 230 | 110 |
| 47 | 75189/76400 | 111 | 403 | 112 |
| 48 | 77794/76460 | 113 | 444 | 114 |
| 49 | 78801/77968 | 115 | 277 | 116 |
| 50 | 78892/79533 | 117 | 213 | 118 |
| 51 | 80344/79544 | 119 | 266 | 120 |
| 52 | 80936/80346 | 121 | 196 | 122 |
| 53 | 81022/81351 | 123 | 109 | 124 |
| 54 | 81348/81776 | 125 | 142 | 126 |
| 55 | 82077/82955 | 127 | 292 | 128 |
| 56 | 82998/84011 | 129 | 337 | 130 |
| 57 | 84224/85282 | 131 | 352 | 132 |
| 58 | 85643/85434 | 133 | 69 | 134 |
| 59 | 87546/85768 | 135 | 592 | 136 |
| 60 | 87826/87647 | 137 | 59 | 138 |
| 61 | 87909/87832 | 139 | 25 | 140 |
| 62 | 88485/87982 | 141 | 167 | 142 |
| 63 | 88571/89350 | 143 | 259 | 144 |
| 64 | 89542/89976 | 145 | 144 | 146 |
| 65 | End [90573]/89980 | 147 | incomplete | 148 |

*involved in primary metabolism

The invention provides nucleic acids that specifically hybridize (or specifically bind) under stringent hybridization conditions to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:11, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149. Also contemplated are nucleic acids that would specifically bind to the aforementioned sequences but for the degeneracy of the nucleic acid code. The nucleic acids can be of sufficient length to encode a complete protein (e.g., a complete orf) or a fragment thereof. Also included are nucleic acids that encode modified proteins. Examples of protein modifications include, but are not limited to, fusions to targeting molecules such as antibodies, antibody fragments, receptor ligands and the like.

The nucleic acids further include probes and primers. In certain embodiments, the probes or primers may be degenerate. Further, in accordance with their use, probes and primers may be single or double stranded. Probes and primers include, for example, oligonucleotides that are at least about 12 nucleotides in length, preferably at least about 15 nucleotides in length, and more preferably at least about 18 nucleotides in length, and further include PCR amplification products that might be generated using primers of the invention.

Hybridization under stringent conditions refers to conditions under which a probe will hybridize preferentially to its target subsequence, and to a lesser extent to, or not at all to, other sequences. It also will be understood that stringent hybridization and stringent hybridization wash conditions in the context of nucleic acid hybridization experiments such as southern and northern hybridizations are sequence dependent, and are different under different environmental parameters. It is well known in the art to adjust hybridization and wash solution contents and temperatures such that stringent hybridization conditions are obtained. Stringency depends on such parameters as the size and nucleotide content of the probe being utilized. See Sambrook et al., 1989, Molecular Cloning—A Laboratory Manual (2nd ed.) Vol. 1-3, Cold Spring Harbor Laboratory, Cold Spring Harbor Press, NY, and other sources for general descriptions and examples. Another guide to the hybridization of nucleic acids is found in Tijssen, 1993, Laboratory Techniques in Biochemistry and Molecular Biology—Hybridization with Nucleic Acid Probes, part I, chapter 2, Overview of principles of hybridization and the strategy of nucleic acid probe assays, Elsevier, N.Y.

Preferred stringent conditions are those that allow a probe to hybridize to a sequence that is more than about 90% complementary to the probe and not to a sequence that is less than about 70% complementary. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2 times SSC wash at 65° C. for 15 minutes (see, Sambrook et al., 1989). Often, a high stringency wash is preceded by a low stringency wash to remove background probe signal. An example of a medium stringency wash for a duplex of, e.g., more than 100 nucleotides, is 1 times SSC at 45° C. for 15 minutes. An example of a low stringency wash for a duplex of, e.g., more than 100 nucleotides, is 4-6 times SSC at 40° C. for 15 minutes. In general, a signal to noise ratio that is two times (or higher) that observed for an unrelated probe in the particular hybridization assay indicates detection of a specific hybridization.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code. Accordingly, nucleotide sequences of the invention include sequences of nucleotides that are at least about 70%, preferably at least about 80%, and more preferably at least about 90% identical to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149 or fragments thereof that are at least about 50 nucleotides, more preferably at least about 100 nucleotides in length.

The present invention is also directed to methods of producing one or more proteins encoded by the chromophore gene cluster. Such proteins may be produced by expressing one or more nucleic acids comprising SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149 in a host cell. For example, one or more of the aforementioned nucleic acids can be operably linked to regulatory control nucleic acids to affect expression, and incorporated into a vector for expression in a host cell. In one embodiment of the invention, the apoprotein or the pre-apoprotein is produced.

Control elements useful in the present invention include promoters, optionally containing operator sequences and ribosome binding sites. Other regulatory sequences may also be desirable, such as those which allow for regulation of expression of apoprotein or pre-apoprotein relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. Various expression vectors are known in the art, e.g., cosmids, P is, YACs, BACs, PACs, HACs.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes that confer antibiotic resistance or sensitivity to the plasmid.

The vectors described above can be inserted in any prokaryotic or eukaryotic cell suitable for protein expression. Host cells include, but are not limited to Actinomadura, Streptomyces, Micromonospora, Actinomyces, Nonomurea, Pseudomonas, and the like. Preferred host cells are those of species or strains (e.g. bacterial strains) that naturally express enediynes such as Actinomadura, Streptomyces, and Micromonospora. (See, e.g., Pfeifer et al., 2001, Science 291, 1790-2; Martinez et al., 2004, Appl. Environ. Microbiol. 70, 2452-63) In one embodiment, the proteins are expressed in E. coli. Recovery of the expression products can be accomplished according to standard methods well known to those of skill in the art. Thus, for example, the proteins can be expressed with a convenient tag to facilitate isolation (e.g., a His₆ tag). Other standard protein purification techniques are suitable and well known to those of skill in the art (see, e.g., Quadri et al., 1998, Biochemistry 37, 1585-95; Nakano et al., 1992, Mol. Gen. Genet. 232, 313-21). When the entire chromoprotein gene cluster is expressed, the chromoprotein can be recovered. By selecting certain orfs for expression, chromoprotein related compounds can be produced. For example, the pre-apoprotein can be produced by expression of orf23.

One may also use a nucleic acid molecule comprising SEQ ID NO:1,

SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149, or a fragment thereof as a probe. Such probes are useful to identify nucleic acids of the invention. One may use the nucleotide sequences as a probe by any suitable method, including a method similar to that described in the Examples below. As described herein, a dNDP-glucose-4,6-dehydratase (DH) probe was used to identify cosmid clones of Actinomadura sp. 21G792 genomic DNA that might contain a gene or gene cluster encoding an apoprotein or other chromophore related proteins. Similarly, the nucleic acids of the invention can be used to identify orfs encoding apoproteins and chromophore related proteins, particularly nine-membered ring enediyne chromophores, in other organisms. Such organisms generally include organisms that produce secondary metabolites, such as, for example, fungi, bacillus, pseudomonads, myxobacteria and cyanobacteria. Preferably, the nucleic acids are used to identify genes of an organism of the order Actinomycetales (Taxonomic Outline of the Procaryotic Genera: Bergey's Manual® of Systematic Bacteriology, 2^{nd} Edition) including but not limited to an organism of the genus Actinomyces, Streptomyces or Micromonospora. More preferably, the nucleic acids are used to identify genes of species and subspecies of Actinomadura.

The present invention also provides substantially pure proteins and polypeptides. The term “substantially pure” as used herein in reference to a given polypeptide means that the polypeptide is substantially free from other biological macromolecules. For example, the substantially pure polypeptide is at least 75%, 80%, 85%, 95%, or 99% pure by dry weight. Purity can be measured by any appropriate standard method known in the art, for example, by column chromatography, polyacrylamide gel electrophoresis, or HPLC analysis. It will be appreciated that substantially pure proteins include chromoproteins, wherein an apoprotein is complexed with an enediyne molecule. Such attachment can be, for example, by a covalent or non-covalent bond, e.g., a hydrogen bond.

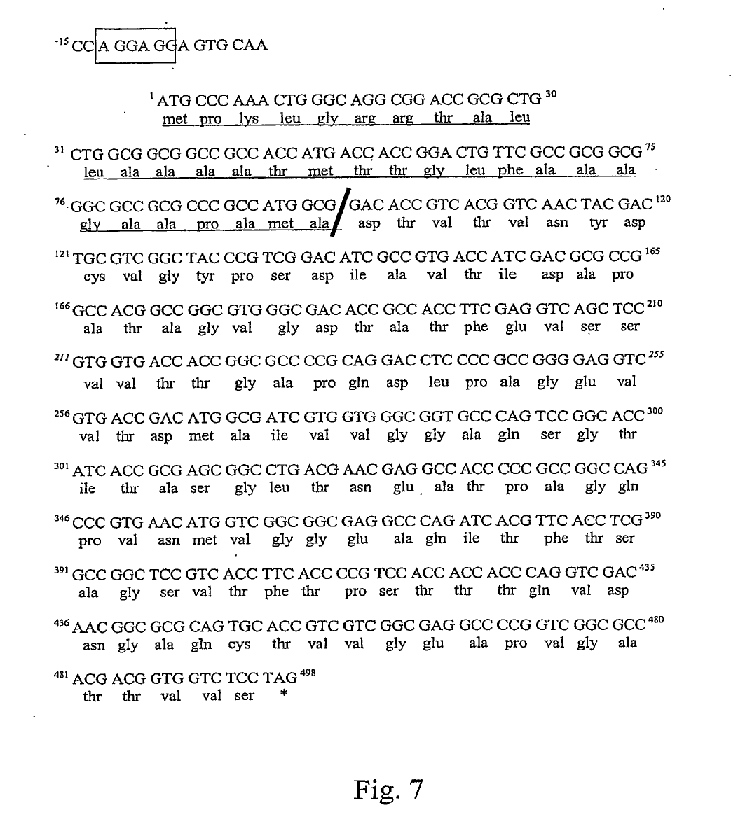

Proteins and polypeptides of the invention include those encoded by the orfs of the chromoprotein gene cluster of Actinomadura sp. 21G792. In preferred embodiments, the proteins and polypeptides are those comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150. In a particular preferred embodiment, the protein is the 21G792 pre-apoprotein (SEQ ID NO:64) or apoprotein (SEQ ID NO:150) (FIG. 7). Amino acid compositions of the 21G792 pre-apoprotein and apoprotein are provided in Table 2.

TABLE 2
Amino Acid Composition of the
Actinomadura sp. 21G792 Apoprotein

| Amino Acid | Number | Composition (%) |
|---|---|---|
| Asp | 8 | 6.02 |
| Asn | 4 | 3.01 |
| Thr | 23 | 17.29 |
| Ser | 9 | 6.77 |
| Glu | 5 | 3.76 |
| Gln | 6 | 4.51 |
| Pro | 8 | 6.02 |
| Gly | 16 | 12.03 |
| Ala | 17 | 12.78 |
| Val | 21 | 15.79 |
| Cys | 2 | 1.50 |
| Met | 2 | 1.50 |
| Ile | 5 | 3.76 |
| Leu | 2 | 1.50 |
| Tyr | 2 | 1.50 |
| Phe | 3 | 2.26 |

It will also be appreciated that proteins or polypeptides of the invention further include those having substantially the same amino acid sequence as the aforementioned preferred proteins and polypeptides. Substantially the same amino acid sequence is defined herein as a sequence with at least about 70%, preferably at least about 80%, and more preferably at least about 90% homology, as determined by the FASTA search method in accordance with Pearson and Lipman, 1988, Proc. Natl. Acad. Sci. USA 85, 2444-8, including sequences that are at least about 70%, preferably at least about 80%, and more preferably at least about 90% identical, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150.

Such proteins have similar activities to those of Actinomadura sp. 21G792, particularly where there are conservative amino acid substitutions. A conservative amino acid substitution is defined as a change in the amino acid composition by way of changing one or more amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter relevant peptide, polypeptide or protein characteristics (e.g., charge, isoelectric point, affinity, avidity, conformation, solubility) or activity. Typical conservative substitutions are selected within groups of amino acids, which groups include, but are not limited to:

(1) hydrophobic: methionine (M), alanine (A), valine (V), leucine (L), isoleucine (1);
(2) hydrophilic: cysteine (C), serine (S), threonine (T), asparagine (N), glutamine (Q);
(3) acidic: aspartic acid (D), glutamic acid (E);
(4) basic: histidine (H), lysine (K), arginine (R);
(5) aromatic: phenylalanine (F), tyrosine (Y) and tryptophan (W);
(6) residues that influence chain orientation: gly, pro. Accordingly, the present invention also embraces apoproteins and polypeptides having similar amino acid compositions to the 21G792 apoprotein, wherein the amino acid sequences are substantially the same as SEQ ID NO:64 or SEQ ID NO:150, particularly where amino acid substitutions are conservative.

The invention provides for changes to one or more orfs of the Actinomadura sp. 21G792 chromoprotein gene cluster, for example, by introduction of one or more random or targeted mutations, deletions, or insertions. In this manner, the chromophore, the apoprotein, or both may be modified in order to create a chromoprotein that exhibits, for example, decreased toxicity, increased potency, or increased stability. It is recognized that certain enediyne chromophores cleave DNA at sites specific to the chromophore. Further, various chromoproteins possess unique proteolytic activities towards histones. Accordingly, manipulation of the Actinomadura sp. 21G792 apoprotein and/or chromophore can also provide a chromoprotein with altered specificity. Alternatively, the apoprotein can be modified to serve as a carrier or delivery vehicle for an active molecule of choice. The invention also provides for a modified Actinomadura sp. 21G792 chromophore or apoprotein/chromophore complex that can be linked to another biological molecule. In one embodiment, the biological molecule provides for specific targeting of chromophore or chromoprotein. Such a biological molecule can be, for example, an antibody or other ligand for a cell surface molecule or receptor.

For example, a nucleic acid encoding an altered Actinomadura sp. 21G792 apoprotein can be inserted into an expression vector and into a host cell, the host cell cultured under conditions suitable for expression of the apoprotein, and the apoprotein recovered from the host cell or culture medium. Preferably, the host cell is capable of producing an enediyne chromophore or other molecule that can form a complex with the altered apoprotein. Examples of such cells include a variety of antibiotic producing organisms of the order Actinomycetales, particularly enediyne producing organisms such as Actinomadura and Streptomyces. Host cells further include common hosts such as E. coli and yeast. Of course, the altered apoprotein can be expressed in Actinomadura sp. 21G792. In one embodiment, the altered apoprotein will be over-expressed in the host cell. If any other endogenous apoprotein is present in the host cell, the altered apoprotein will be expressed at a higher level, the other apoprotein will be under-expressed, or the altered apoprotein will be expressed with a tag to facilitate such purification. In a preferred embodiment, the nucleic acid encoding the altered apoprotein is substituted for the endogenous apoprotein gene by homologous recombination. As such, the altered apoprotein can then be isolated in a complex with an enediyne or other molecule, e.g., an active agent, and then such a complex can be screened, e.g., against a cancer cell line, to determine bioactivity.

In yet another embodiment, a) the altered apoprotein is expressed in the host cell and is recovered without being complexed to an enediyne or other molecule, b) the altered apoprotein is then subjected to various enediyne or other molecules, c) an acceptable technique is used to determine whether the apoprotein forms a complex with the enediyne or other molecules, and optionally d) the complex is screened for bioactivity. In yet another embodiment, the altered apoprotein is expressed in the host cell and is recovered without being complexed to an enediyne or other molecule, the altered apoprotein is then subjected to various enediyne or other molecules, and the complex is screened for bioactivity.

In another example, nucleic acids encoding a modified chromophore biosynthetic pathway are expressed.

Functions of polypeptides expressed from the Actinomadura sp. 21G792 biosynthetic cluster may be deduced by comparing ORF sequences with known proteins and sequence motifs. (Table 3)

TABLE 3
DEDUCED FUNCTIONS FOR THE ORFS OF THE 21G792
CHROMOPROTEIN GENE CLUSTER

| | | | Access. No.^{c}, | |
|---|---|---|---|---|
| ORF | Size^{a} | Similar Protein | (% id./% sim.) | Proposed Function |
| Orf9* | 462^{b} | ATP synthase beta | AAU08241, n/a | primary metabolism |
| | | subunit, AtpD, | | |
| | | Nonomuraea sp. ATCC | | |
| | | 39727 | | |
| Orf8* | 128 | ATP synthase epsilon | AAU08242, | primary metabolism |
| | | chain, AtpC, | 57/73 | |
| | | Nonomuraea sp. ATCC | | |
| | | 39727 | | |
| Orf7* | 151 | putative membrane | BAC70590, | primary metabolism |
| | | protein, Streptomyces | 44/57 | |
| | | avermitilis MA-4680 | | |
| Orf6* | 532 | probable | AAZ56436, | primary metabolism |
| | | aminopeptidase, | 45/61 | |
| | | Thermobifida fusca YX | | |
| Orf5* | 211 | cobalamin | AAZ56437, | primary metabolism |
| | | adenosyltransferase, | 65/77 | |
| | | Thermobifida fusca YX | | |
| Orf4* | 525 | GMC oxidoreductase, | AAF10542, | primary metabolism |
| | | Deinococcus radiodurans | 49/60 | |
| | | R1 | | |
| Orf3* | 68 | hypothetical protein, | BAD81225, | primary metabolism |
| | | Oryza sativa | 41/52 | |
| Orf2* | 162 | acetyltransferases, | ZP_00132424, | primary metabolism |
| | | Haemophilus somnus | 42/55 | |
| | | 2336 | | |
| Orf1* | 498 | aldehyde | ZP_00657819, | primary metabolism |
| | | dehydrogenase, | 57/73 | |
| | | Nocardioides sp. JS614 | | |
| Orf1 | 331 | unknown, NcsE2, | AAM78016, | unknown |
| | | Streptomyces | 62/69 | |
| | | carzinostaticus | | |
| Orf2 | 300 | unknown, MadE3, | AAQ17107, | unknown |
| | | Actinomadura madurae | 100/100 | |
| Orf3 | 678 | unknown, MadE4, | AAQ17108, | unknown |
| | | Actinomadura madurae | 99/99 | |
| Orf4 | 330 | unknown, MadE5, | AAQ17109, | unknown |
| | | Actinomadura madurae | 100/100 | |
| Orf5 | 1944 | Type I PKS, MadE, | AAQ17110, | Iterative type I |
| | | Actinomadura madurae | 99/99 | PKS: KS, AT, ACP, |
| | | | | DH, KR, TD |
| Orf6 | 152 | putative thioesterase, | AAQ17111, | thioesterase |
| | | MadE10, Actinomadura | 100/100 | |
| | | madurae | | |
| Orf7 | 183 | putative oxidoreductase, | AAQ17112, | oxidoreductase |
| | | MadE6, Actinomadura | 100/100 | |
| | | madurae | | |
| Orf8 | 464 | putative P450 | AAQ17113, | P450 hydroxylase |
| | | hydroxylase, MadE7, | 99/99 | |
| | | Actinomadura madurae | | |
| Orf9 | 251 | transcriptional regulator, | AAM78008, | AraC family, |
| | | NcsR5, Streptomyces | 52/65 | transcriptional |
| | | carzinostaticus | | regulator |
| Orf10 | 336 | transcriptional regulator | BAC53615, | StrR-like |
| | | protein, KasT, | 49/63 | transcriptional |
| | | Streptomyces | | regulator |
| | | kasugaensis | | |
| Orf11 | 218 | putative regulatory | AAL06694, | unknown |
| | | protein, SgcR1, | 58/72 | |
| | | Streptomyces | | |
| | | globisporus | | |
| Orf12 | 552 | oxidoreductase, NcsE9, | AAM78005, | oxidoreductase |
| | | Streptomyces | 79/87 | |
| | | carzinostaticus | | |
| Orf13 | 365 | unknown, SgcM, | AAL06686, | unknown |
| | | Streptomyces | 46/52 | |
| | | globisporus | | |
| Orf14 | 261 | unknown, NcsE11, | AAM78004, | unknown |
| | | Streptomyces | 61/73 | |
| | | carzinostaticus | | |
| Orf15 | 229 | O-methyltransferase, | ZP_00573484, | O- |
| | | Frankia sp. EAN1pec | 49/67 | methyltransferase |
| Orf16 | 95 | NRPS PCP-domain, | BAB69396, | aryl carrier protein |
| | | NRPS7-5, Streptomyces | 41/53 | |
| | | avermitilis MA-4680 | | |
| Orf17 | 1120 | type II NRPS A domain, | AAL06681, | NRPS: C, A, PCP |
| | | SgcC1, Streptomyces | 41/49 | |
| | | globisporus | | |
| Orf18 | 537 | aminomutase, SgcC4, | AAL06680, | aminomutase |
| | | Streptomyces | 73/84 | |
| | | globisporus | | |
| Orf19 | 548 | putative halogenase, | ZP_00548729, | halogenase |
| | | Frankia sp. Ccl3 | 62/75 | |
| Orf20 | 460 | type II NRPS C domain, | AAL06678, | type II NRPS C |
| | | SgcC5, Streptomyces | 46/59 | domain |
| | | globisporus | | |
| Orf21 | 442 | squalene | AAL06669, | monooxygenase |
| | | monooxygenase-like | 50/56 | |
| | | protein, SgcD2, | | |
| | | Streptomyces | | |
| | | globisporus | | |
| Orf22 | 525 | transmembrane efflux | AAF13999, | transmembrane |
| | | protein, SgcB, | 48/67 | efflux protein |
| | | Streptomyces | | |
| | | globisporus | | |
| Orf23 | 165 | hypothetical protein, | BAC71199, | pre-apoprotein |
| | | Streptomyces avermitilis | 33/44 | |
| | | MA-4680 | | |
| Orf24 | 461 | adenosylmethionine-8- | BAD39928, | aminotransferase |
| | | amino-7-oxononanoate | 43/58 | |
| | | aminotransferase, | | |
| | | Symbiobacterium | | |
| | | thermophilum | | |
| Orf25 | 408 | P450 hydroxylase, | BAC75180, | P450 hydroxylase |
| | | Cyp28, Streptomyces | 45/59 | |
| | | avermitilis MA-4680 | | |
| Orf26 | 381 | hypothetical protein, | CAC22728, | unknown |
| | | Streptomyces coelicolor | 33/46 | |
| | | A3(2) | | |
| Orf27 | 409 | putative cytochrome | AAC25766, | P450 |
| | | P450 oxidoreductase, | 45/60 | oxidoreductase |
| | | Streptomyces lividans | | |
| | | 1326 | | |
| Orf28 | 232 | conserved hypothetical | AD63964, | unknown |
| | | protein, Bacillus clausii | 51/71 | |
| | | KSM-K16 | | |
| Orf29 | 466 | glycosyltransferase, | AAL06670, | glycosyltransferase |
| | | SgcA6, Streptomyces | 43/57 | |
| | | globisporus | | |
| Orf30 | 285 | putative hydrolase, | BAC69810, | epoxide hydrolase |
| | | Streptomyces avermitilis | 39/52 | |
| | | MA-4680 | | |
| Orf31 | 548 | putative salicyl-AMP | BAC78380, | aryl acid-AMP |
| | | ligase, SdgA, | 54/64 | ligase |
| | | Streptomyces sp. WA46 | | |
| Orf32 | 1746 | type I PKS, NcsB, | AAM77986, | iterative type I PKS: |
| | | Streptomyces | 47/59 | KS, AT, DH, KR, |
| | | carzinostaticus | | ACP |
| Orf33 | 348 | O-methyltransferase, | ZP_00671263, | C- |
| | | Trichodesmium | 35/55 | methyltransferase |
| | | erythraeum | | |
| Orf34 | 393 | oxidoreductase, SgcL, | AAB13590, | oxidoreductase |
| | | Streptomyces | 67/78 | |
| | | globisporus | | |
| Orf35 | 148 | unknown, SgcT, | AAL06676, | unknown |
| | | Streptomyces | 61/76 | |
| | | globisporus | | |
| Orf36 | 401 | probable | AAG23279, | aminotransferase |
| | | aminotransferase, | 55/68 | |
| | | SpnR, | | |
| | | Saccharopolyspora | | |
| | | spinosa | | |
| Orf37 | 447 | UDP-glucose | AAM70332, | NDP-glucose |
| | | dehydrogenase CalS8, | 52/63 | dehydrogenase |
| | | Micromonospora | | |
| | | echinospora | | |
| Orf38 | 328 | CalS9, Micromonospora | AAM70333, | NDP-glucuronate |
| | | echinospora | 61/71 | decarboxylase |
| Orf39 | 539 | chlorophenol-4- | AAL06674, | aromatic ring |
| | | monooxygenase, SgcC, | 73/82 | hydroxylase |
| | | Streptomyces | | |
| | | globisporus | | |
| Orf40 | 406 | putative C-3 methyl | CAC48364, | C- |
| | | transferase, DvaC, | 58/74 | methyltransferase |
| | | Amycolatopsis | | |
| | | balhimycina | | |
| Orf41 | 340 | alcohol dehydrogenase, | AAK90613, | alcohol |
| | | Agrobacterium | 55/71 | dehydrogenase |
| | | tumefaciens str. C58 | | |
| Orf42 | 460 | squalene | AAL06669, | monooxygenase |
| | | monooxygenase-like | 60/72 | |
| | | protein, SgcD2, | | |
| | | Streptomyces | | |
| | | globisporus | | |
| Orf43 | 347 | NDP-1-glucose | BAC79029, | dNDP-glucose |
| | | synthase, med-ORF18, | 55/71 | synthase |
| | | Streptomyces sp. AM- | | |
| | | 7161 | | |
| Orf44 | 138 | putative lyase, | CAC37263, | lyase |
| | | Streptomyces coelicolor | 47/61 | |
| | | A3(2) | | |
| Orf45 | 557 | putative methylmalonyl- | BAC70414, | carboxylyase/carboxyl |
| | | CoA decarboxylase | 66/79 | transferase, |
| | | alpha subunit, MmdA2, | | lipid metabolism |
| | | Streptomyces avermitilis | | |
| | | MA-4680 | | |
| Orf46 | 230 | possible trancriptional | CAD93534, | TetR family, |
| | | regulator, | 37/50 | transcriptional |
| | | Mycobacterium bovix | | regulator |
| Orf47 | 403 | retinal pigment epithelial | ZP_00577676, | dioxygenase |
| | | membrane protein, | 31/40 | |
| | | Sphingopyxis alaskensis | | |
| | | RB2256 | | |
| Orf48 | 444 | putative dioxygenase, | AAK06796, | dioxygenase |
| | | SimC5, Streptomyces | 43/53 | |
| | | antibioticus | | |
| Orf49 | 277 | conserved hypothetical | AAZ55273, | dNDP-sugar |
| | | protein, Thermobifida | 51/64 | epimerase |
| | | fusca YX | | |
| Orf50 | 213 | transcriptional regulatory | BAC49474, | TetR family, |
| | | protein, Bradyrhizobium | 45/60 | transcriptional |
| | | japonicum | | regulator |
| Orf51 | 266 | putative membrane | CAB61706, | unknown |
| | | protein, Streptomyces | 52/66 | |
| | | coelicolor A3(2) | | |
| Orf52 | 196 | putative TetR-family | CAB71239, | TetR family, |
| | | transcriptional regulator, | 30/47 | transcriptional |
| | | Streptomyces coelicolor | | regulator |
| | | A3(2) | | |
| Orf53 | 109 | transcriptional regulator, | BAB53793, | ArsR family, |
| | | Mesorhizobium loti | 50/70 | transcriptional |
| | | | | regulator |
| Orf54 | 142 | conserved hypothetical | CAD17332, | unknown |
| | | protein, Ralstonia | 49/58 | |
| | | solanacearum | | |
| Orf55 | 292 | LysR family regulatory | ZP_00571435, | LysR family, |
| | | protein, Frankia sp. | 43/54 | transcriptional |
| | | EAN1pec | | regulator |
| Orf56 | 337 | class A beta-lactamase, | AAG44836, | unknown |
| | | Bla, Nocardia asteroides | 46/58 | |
| Orf57 | 352 | hypothetical protein, | ZP_00667098, | unknown |
| | | Syntrophobacter | 26/40 | |
| | | fumaroxidans | | |
| Orf58 | 69 | none | — | unknown |
| Orf59 | 592 | RNA-directed DNA | ZP_00570947, | unknown |
| | | polymerase, Frankia sp. | 70/80 | |
| | | EAN1pec | | |
| Orf60 | 59 | none | — | unknown |
| Orf61 | 25 | none | — | unknown |
| Orf62 | 167 | putative regulatory | CAC44216, | regulator |
| | | protein, Streptomyces | 40/47 | |
| | | coelicolor A3(2) | | |
| Orf63 | 259 | conserved hypothetical | CAB62713, | unknown |
| | | protein, Streptomyces | 32/50 | |
| | | coelicolor A3(2) | | |
| Orf64 | 144 | NUDIX hydrolase, | ZP_00572338, | DNA repair |
| | | Frankia sp. EAN1pec | 38/56 | |
| Orf65 | 197^{b} | putative binding-protein- | CAE50656, n/a | ABC transporter |
| | | dependent integral | | |
| | | membrane protein, | | |
| | | Corynebactrium | | |
| | | diphtheriae | | |

^{a}Numbers are in amino acids
^{b}Incomplete Orf
^{c}NCBI accession numbers of closest homologs are given
*Involved in primary metabolism

Consistent with those functions, a convergent biosynthetic pathway is provided for synthesis of the Actinomadura sp. 21G792 enediyne. Four primary components of the complex (enediyne core, madurosamine, 2-hydroxy-3,6-dimethyl benzoic acid, and 3-(2-chloro-3-hydroxy-4-methoxyphenyl)-3-hydroxy-propanoic acid) are produced separately and then assembled to form the final bioactive product.

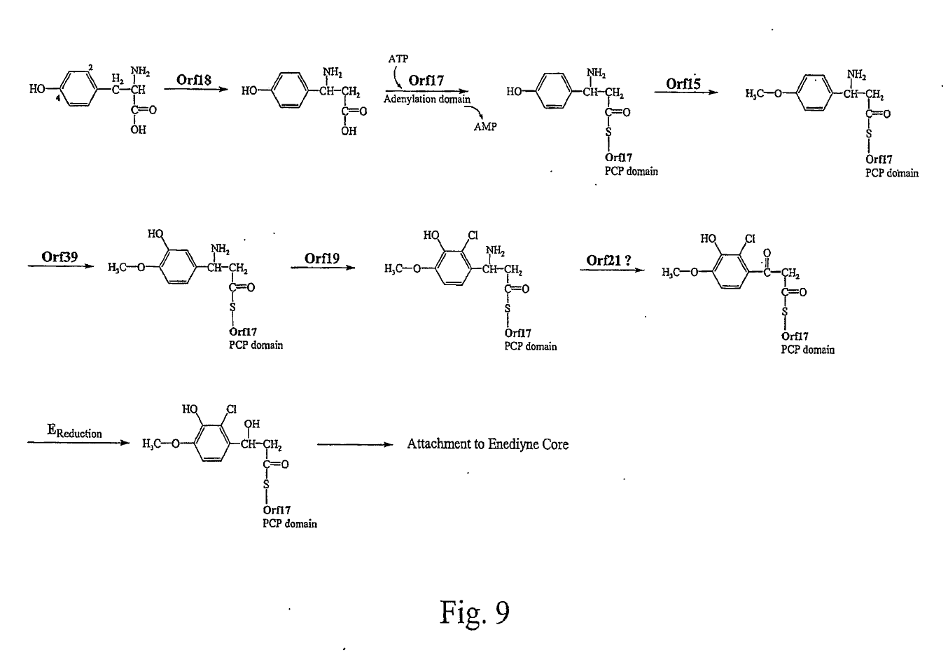

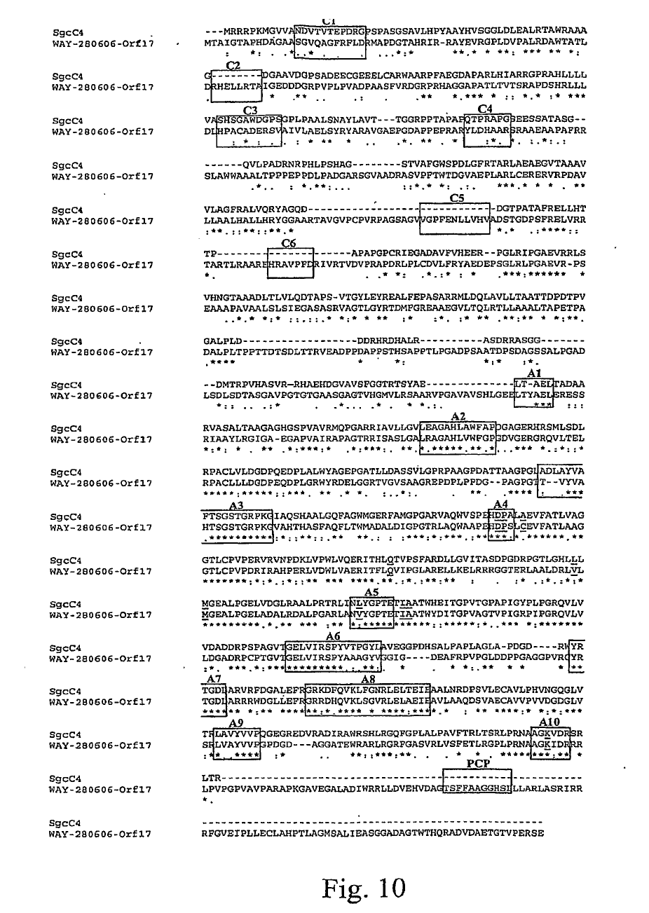

3-(2-chloro-3-hydroxy-4-methoxy-phenyl)-3-hydroxy-propanoic acid moiety biosynthesis. To produce the 3-(2-chloro-3-hydroxy-4-methoxy-phenyl)-3-hydroxy-propionic acid-derived portion of the enediyne (FIG. 9), tyrosine is first converted to β-tyrosine by the gene product of orf18. Orf18 shows high similarity to several histidine and phenylalanine ammonia lyases, but is most similar to SgcC4 of the C-1027 biosynthetic pathway (73% identity, 84% similarity), which catalyzes the conversion of β-tyrosine to β-tyrosine. (Liu et al., 2002, Science, 297, 1170-73, Van Lanen et al., 2005, J. Am. Chem. Soc., 127, 11594-5). Next, β-tyrosine is activated as an aminoacyl adenylate by the adenylation (A) domain of the orf17 gene product, and transferred to the sulfhydryl group of the phosphopantetheinyl prosthetic group on the adjacent peptidyl carrier protein (PCP), forming β-tyrosinyl-S-Orf17. Orf17 is similar to a wide array of nonribosomal peptide synthetases (NRPSs). Based on sequence analysis of the deduced amino acid sequence, Orf17 comprises three functional domains, a condensation (C) domain, an A domain and a PCP domain (FIG. 10). See, Konz and Marahiel, 1999, Chem. Biol., 6, R39-R47. The substrate specificity code of the A domain was extracted from the region between the A4 and A5 A domain structural motif, revealing the specificity code DPCQVMVIAK (Table 4). Table 4 also depicts the substrate and substrate specificity codes for SgcC1 from the C1027 biosynthetic cluster (Challis et al., 2000, Chem. Biol. 7, 211-24) and GrsA from the gramicidin biosynthetic cluster (Stachelhaus et al., 1999, Chem. Biol., 6, 493-505).

TABLE 4
COMPARISON OF ADENYLATION DOMAIN SUBSTRATE SPECIFICITY CODES

| | Amino Acid Position (GrsA numbering) | | | | | | | | | | |
|---|---|---|---|---|---|---|---|---|---|---|---|
| | 235 | 236 | 239 | 278 | 299 | 301 | 322 | 330 | 331 | 517 | Substrate |
| GrsA | D | A | W | T | I | A | A | I | C | K | Phe |
| Orf17 | D | P | C | Q | V | M | V | I | A | K | β-Tyr |
| SgcC1 | D | P | A | Q | L | M | L | I | A | K | β-Tyr |

Orf17 is most similar to SgcC1 from the C-1027 biosynthetic cluster (41% identity, 49% similarity). SgcC1 encodes a type II non-ribosomal peptide synthetase (NRPS) that is composed of a lone A domain. In vitro characterization of the enzyme has shown that it specifically activates β-tyrosine prior to loading it on SgcC2, a type II NRPS composed of a single PCP domain. (Van Lanen et al., 2005). Comparison of the substrate specificity codes of SgcCl and Orf17 reveals that the codes are remarkably similar (DPCQVMVIAK for Orf17 versus DPAQLMLIAK for SgcCl). This similarity is not surprising as both enzymes activate the same substrate. Interestingly, the stop codon of orf17 overlaps the start of orf18 by 3 bp, indicating that the expression of these two genes might be translationally coupled. Coordinating the expression of these genes is not unexpected, as expression of orf17 without the concurrent expression of orf18 to supply β-tyrosine, would result in the production of the orf17 gene product without a supply of its intended substrate.

Once loaded on the PCP of Orf17 via a thioester linkage, β-tyrosinyl-S-Orf17 is next methylated by Orf15 to give 3-amino-3-(4-methoxy-phenyl)-propanyl-S-Orf17. Orf15 shows strong similarity to many S-adenosylmethionine (SAM)-dependent O-methyltransferases and possesses three sequence motifs common to SAM-dependent methyltransferases (Motif I-VVDVGTFTG, SEQ ID NO:166; Motif 2-PAADLVFL, SEQ ID NO:167; Motif 3-LLRPGGLLVA, SEQ ID NO:168). Kagan and Clarke, (1994) Arc. Biochem. Biophys., 310, 417-427. As Actinomadura sp. 21G792 enediyne possesses a single O-methyl group, Orf15 is the enzyme most likely to catalyze this reaction. This enzyme-tethered intermediate is subsequently hydroxylated by Orf39 to yield 3-amino-3-(3-hydroxy-4-methoxy-phenyl)-propanyl-S-Orf17. BlastP analysis indicates that Orf39 is a hydroxylase similar to many hydroxylases responsible for the hydroxylation of phenolic substrates. It is strikingly similar to SgcC of the C-1027 biosynthetic cluster (73% identity, 82% similarity), which was shown, in vitro, to hydroxylate a chlorinated □-tyrosinyl-S-PCP intermediate. (Liu et al, 2002; Van Lanen et al., 2005). Following hydroxylation, the orf19 gene product chlorinates the C-2 position of the aromatic ring to yield 3-amino-3-(2-chloro-3-hydroxy-4-methoxy-phenyl)-propanyl-S-Orf17. Orf19 is homologous to several alkyl halidases involved in secondary metabolism, most notably SgcC3 from the C-1027 biosynthetic cluster (58% identity, 70% similarity), which has been shown to perform the chlorination of PCP bound β-tyrosine. (Liu et al, 2002; Van Lanen et al., 2005).

Since the β-tyrosine derivative incorporated into the Actinomadura sp. 21G792 enediyne bears a hydroxyl group in place of the amino group, one can envision the amino group of the 3-amino-3-(2-chloro-3-hydroxy-4-methoxy-phenyl)-propanyl-S-Orf17 intermediate being replaced by Orf21 via oxidative deamination. BlastP analysis reveals that Orf21 shows similarity to several putative FAD and NADPH-dependant monooxygenases/hydroxylases and domain analysis shows that it contains an FAD binding domain common to many monooxygenases. This domain is common to amino acid oxidases where oxidative deamination is well documented, thus Orf21 is a likely candidate to perform this transformation. It is important to note however, that there are several other candidates that could potentially catalyze this reaction including Orf42, which is also similar to FAD and NADPH-dependant monooxygenases/hydroxylases. Additionally, two Orfs (Orf25 and Orf27), which are similar to P450 hydroxylases, are present in the biosynthetic cluster and as P450 hydroxylases have also been implicated in oxidative deamination reactions, one of these enzymes might also catalyze this step. (Li et al., 2000, J. Bacteriol. 182, 4087-95) Following oxidative deamination, reduction of the ketone likely introduced by Orf21 or one of the other candidate enzymes, is likely to occur. The most obvious enzyme capable of catalyzing such a reaction would be a ketoreductase, similar to those employed in polyketide biosynthesis. Examination of the Actinomadura sp. 21G792 enediyne biosynthetic cluster did not identify any enzymes showing similarity to ketoreductase-like enzymes. There are several enzymes in the cluster that have unknown functions that might catalyze the required reduction, or the enzyme responsible for catalyzing the oxidative deamination might also catalyze the reduction reaction. Alternatively, an enzyme encoded outside of the current biosynthetic pathway could catalyze the expected reduction. Following ketoreduction the tyrosine derivative 3-(2-chloro-3-hydroxy-4-methoxy-phenyl)-3-hydroxy-propanyl-S-Orf17, is ready to be incorporated into the Actinomadura sp. 21G792 enediyne complex. The incorporation of this component of the Actinomadura sp. 21G792 enediyne into the final product is discussed below.

This synthetic pathway is not considered limiting but merely illustrative. Using this as a model, one of ordinary skill in the art can design numerous other synthetic schemes to produce the 3-(2-chloro-3-hydroxy-4-methoxy-phenyl)-3-hydroxy-propanyl component of the Actinomadura sp. 21G792 chromophore or a derivative of this component.

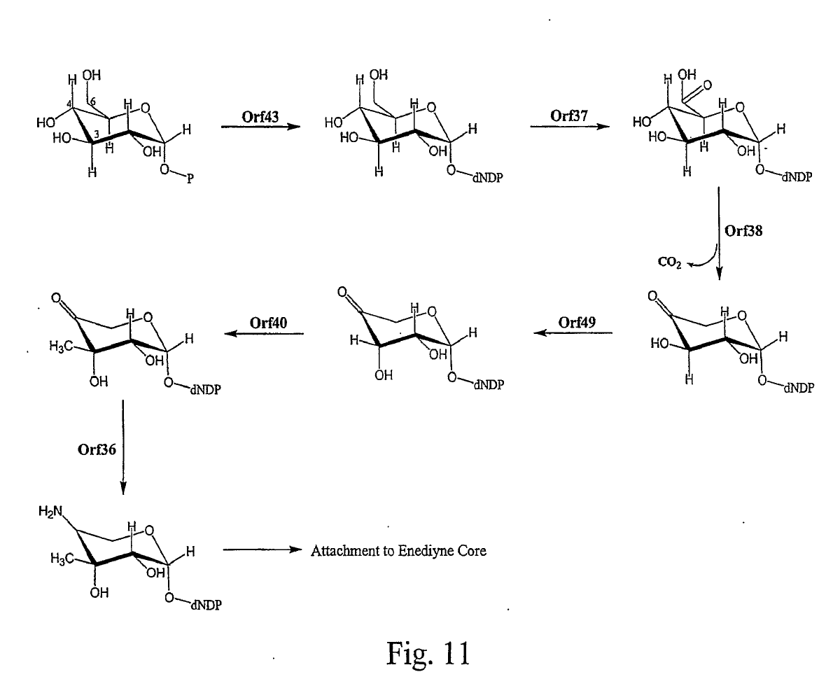

Madurosamine moiety biosynthesis. Analysis of the Actinomadura sp. 21G792 enediyne biosynthetic pathway identified five genes likely involved in madurosamine (4-amino-4-deoxy-3-C-methyl-β-ribopyranose) biosynthesis (FIG. 11). The first step in madurosamine (MDA) biosynthesis, as with all deoxysugars, is activation of D-glucose-1-phosphate (G-1-P) by a glucose-dNDP synthase. Trefzer et al., 1999, Nat. Prod. Rep. 16, 283-99. Orf43, which is homologous to several glucose-dNDP synthases, is responsible for activating G-1-P. Based on sequence homology of Orf43 to other proteins in the GenBank database, it likely catalyzes the formation of dTDP or dUDP-glucose.

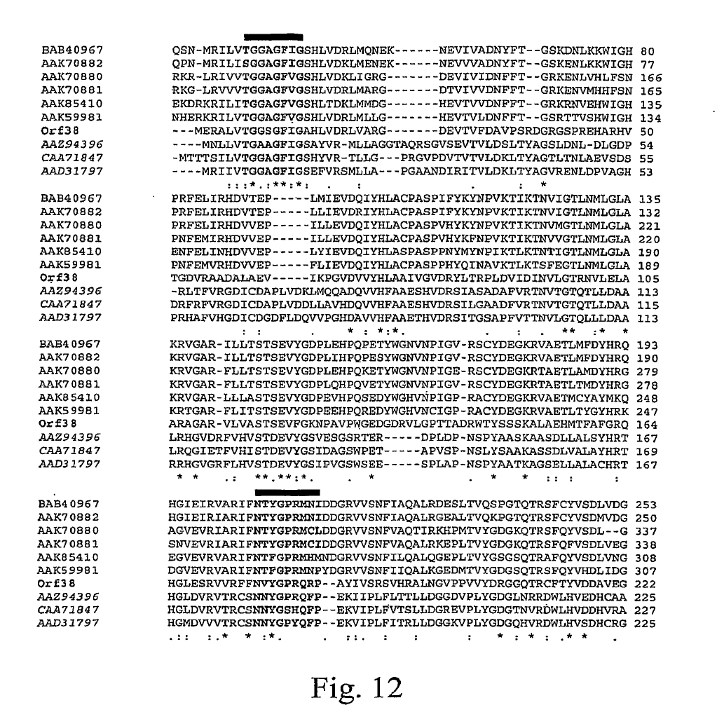

Next, Orf37, an enzyme highly homologous to dNDP-sugar dehydrogenases, oxidizes the primary alcohol to an acid, producing dNDP-D-glucuronate. Orf38, a probable dNDP-glucuronate decarboyxlase, then converts dNDP-D-glucuronate to dNDP-xylose. A fragment amplified from orf38 was used as a probe to identify the first cosmid containing the Actinomadura sp. 21G792 enediyne biosynthetic cluster (See Examples) based on the prediction that biosynthesis of madurosamine might involve a dNDP-glucose-4,6-dehydratase including a 4,6-deoxyglucose intermediate. However, comparison of UDP-glucuronate decarboxylase and TDP-glucose-4,6-dehydratase amino acid sequences to that of Orf38 shows that the conserved amino acid motifs used by Decker et al. to design PCR primers used to amplify glucose-4,6-dehydratase genes, are also present in Orf38 and in the glucuronate decarboxylase sequences (FIG. 12). (Decker et al., 1994, FEMS Micro. Lett., 141, 195-201). Consequently it is not surprising that a glucuronate decarboxylase was amplified using these primers. Additionally, it should be noted that the stop codon of orf37 overlaps with the start codon of orf38, indicating that these orfs might be translationally coupled.

Following decarboxylation of dNDP-glucuronate, the C-3 hydroxyl of dNDP-D-xylose is epimerized by Orf49, producing dNDP-L-xylose. Orf49 is most similar to an uncharacterized protein from Thermobifida fusca (Accession no. AAZ55273.1) and its next most closely related homolog is ovmX (40% identity, 53% similarity), a putative NDP-sugar epimerase from Streptomyces antibioticus ATCC 11891 involved in the biosynthesis of oviedomycin. (Lombo et al., 2004, Chembiochem 5, 1181-7)

Following epimerization, the gene product of orf40 methylates the 3-carbon of dNDP-L-xylose. Orf40 shows significant similarity to a number of NDP-hexose C-methyltransferases and possesses three sequence motifs common to a wide variety of SAM dependent methyltransferases (Motif 1-IVEIGCNDG, SEQ ID NO:169; Motif 2-GPADVLYG, SEQ ID NO:170; Motif 3-LLKPDGIFVF, SEQ ID NO:171). (Kagan and Clarke, 1994, Arc. Biochem. Biophys., 310, 417-27). As a result, Orf40 is expected to perform this methylation. While another C-methylation is expected to occur in the biosynthesis of the 2-hydroxy-3,6-dimethyl-benzoic acid (HDBA) moiety of the Actinomadura sp. 21G792 enediyne, the C-methyltransferase expected to catalyze that methylation (Orf33), appears to form a small operon with the polyketide synthase responsible for generating the HDBA carbon skeleton, consequently Orf40 is not expected to participate in that transformation.

The methylated dNTP-sugar next undergoes C-4 transamination to form dNTP-madurosamine. This reaction is likely catalyzed by Orf36, which is highly homologous to SpnR (55% identity, 68% similarity) from the spinosyn biosynthetic cluster, which has been shown to carry out the C-4 transamination of a deoxysugar intermediate in the formation of D-forosamine. (Zhao et al., 2005, JACS, 127, 7692-3) The incorporation of the madurosamine component of Actinomadura sp. 21G792 enediyne into the final product will be discussed below.

This synthetic pathway is not considered limiting but merely illustrative. Using this as a model, one of ordinary skill in the art can design numerous other synthetic schemes to produce the MDA component of Actinomadura sp. 21G792 enediyne or a derivative of this component.

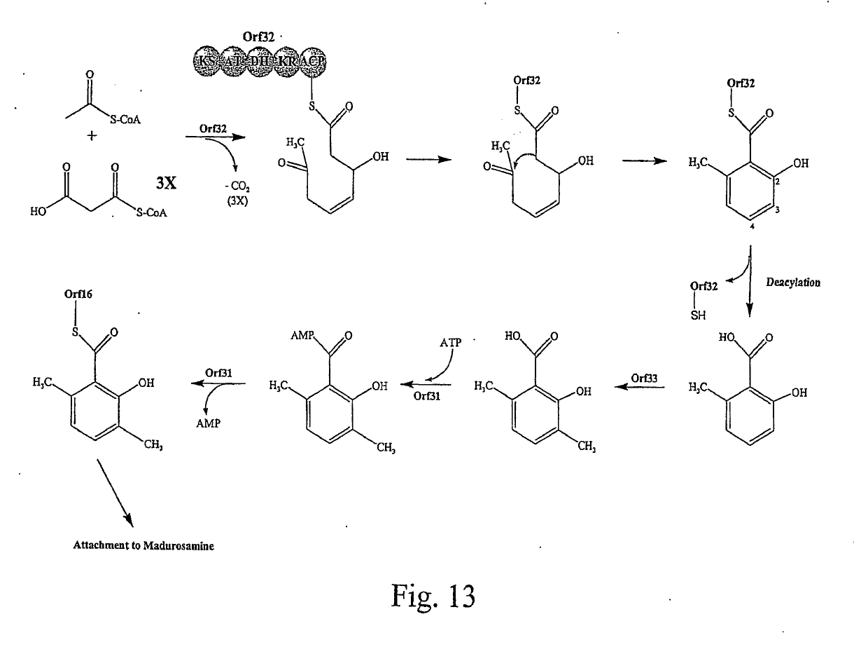

2-Hydroxy-3,6-dimethyl-benzoic acid moiety biosynthesis. The 2-hydroxy-3,6-dimethyl benzoic acid (HDBA) component of Actinomadura sp. 21G792 enediyne is most likely synthesized by two gene products, Orf32 an iterative type I polyketide synthase (PKS) and Orf33, a SAM-dependent C-methyltransferase (FIG. 13). Until recently, the bacterial paradigm for the biosynthesis of aromatic polyketides called for an iterative type II PKS. (Shen et. al., 2003, Curr. Opin. Chem. Biol. 7, 285-95) Examination of the Actinomadura sp. 21G792 enediyne biosynthetic cluster did not reveal the presence of any genes homologous to type II PKSs. Orf32, however, showed significant similarity to NcsB (47% identity, 59% similarity), an iterative type I PKS responsible for the production of the napthoic acid moiety of neocarzinostatin and to several 6-methylsalicylic acid synthases of fungal origin. (Liu et al., 2005, Chem. Biol., 293-302) Orf32 consists of 5 domains common to type I PKSs including a ketosynthase (KS), acyltransferase (AT), dehydratase (DH), ketoreductase (KR) and acyl carrier protein (ACP). It catalyzes the formation of a linear tetraketide from one acetyl-coenzyme A (coA) and 3 malonyl-coAs by iterative decarboxylative condensation followed by selective ketoreduction and dehydration at C-4 and ketoreduction at C-2. The nascent tetraketide intermediate then undergoes a nonenzymatic intramolecular aldol condensation to form the cyclized, 6-methylsalicylic (6MSA) acid intermediate.

The gene product of orf33 subsequently methylates the C-3 position of the 6MSA intermediate to form HDBA. Orf33 is similar to a wide variety of SAM-dependent methyltransferases including N-, C- and O-methyltransferases. Consistent with its classification, Orf33 possesses three sequence motifs common to a wide variety of SAM-dependent methyltransferases (Motif 1-VLDLGGGDG, SEQ ID NO:172; Motif 2-DGCDAILY, SEQ ID NO:173; Motif 3-ALPEGGVCVV, SEQ ID NO:174). (Kagan and Clarke, 1994) While the other methyltransferases present in the biosynthetic cluster might catalyze this reaction, Orf33 is immediately upstream of Orf32 and appears to be part of a small operon devoted to the production of HDBA and as a result, is the enzyme most likely to perform this reaction. Release of the cyclized polyketide from the PKS does not require a thioesterase, as is the case with most polyketides. Rather, it is released via a ketene pathway, analogous to that reported for 6-methylsalicylic acid biosynthesis. Spencer and Jordan, (1992) Biochem. J., 288, 839-846.

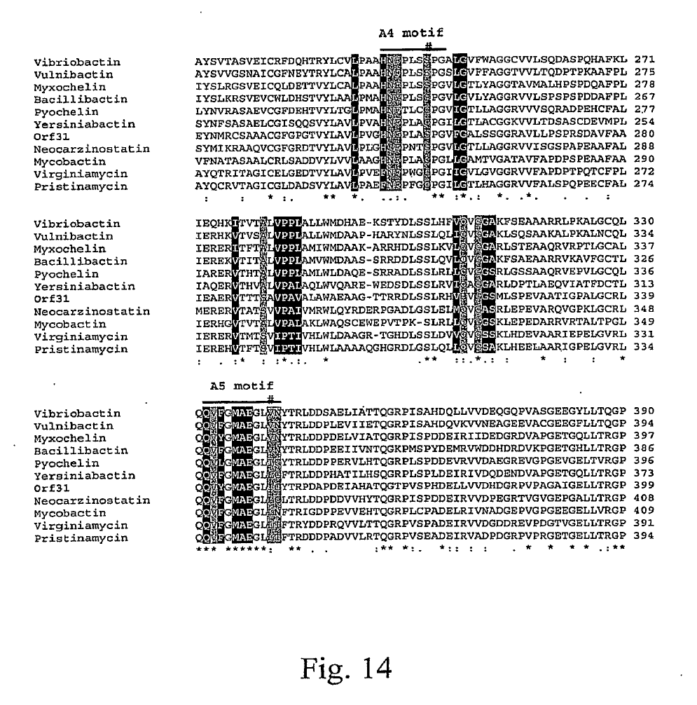

Following release from Orf32, HDBA is activated as an aryl adenylate by the gene product of orf31. Orf31 is similar to a number of aryl acid AMP-ligases. The best-studied examples of these types of enzymes come from investigations into siderophore biosynthesis. In the case of many siderophores, an aryl acid such as salicylate or 2′,3′-dihydroxybenzoate is adenylated as a first step in the assembly of the nonribosomal peptide core of the siderophore (see, Crosa and Walsh, 2002, Microbiol Mol. Biol. Rev., 66, 223-49 for a review). In addition to activating the aryl acid as an adenylate, these enzymes also transfer the aryl acids to the sulfhydryl group of the phosphopantetheinyl prosthetic group of a so-called aryl carrier protein (ArCP). Comparison of the crystal structure of the 2′,3′-dihydroxybenzoate-AMP ligase (DhbE) involved in the biosynthesis of the siderophore bacillibactin to that of other adenylating enzymes, including the NRPS GrsA adenylation domain and firefly luciferase revealed that aryl acid-activating domains contain a signature sequence not present in amino-acid activating domains. (May et al., 2002, PNAS 99, 12120-5). In DhbE, the so-called core A4 motif normally present in amino acid-activating domains (YxFDxS), is replaced by the sequence motif HNYPLSSPG. In amino acid-activating domains the invariant Asp residue stabilizes the □-amino group of the amino acid substrate, while in aryl acid-activating domains, the Asp residue is replaced with the conserved neutral Asn, which hydrogen bonds with the 2′-hydroxyl group of DHBA or salicylic acid. (May et al., 2002). As HDBA possesses a 2′-hydroxyl, one would expect Orf31 to possess the aryl acid-activating A4 motif. Examination of the Orf31 sequence revealed the motif HNFPLASPG (SEQ ID NO:175), which is consistent with enzymes activating aryl acids (FIG. 14).

As for amino acid-activating domains of NRPSs (Stachelhaus et al., 1999, Chem. Biol., 6, 493-505; Challis et al., 2000, Chem. Biol. 7, 211-24), a substrate specificity code for aryl acid-activating domains can be extracted from the region between the A4 and A5 core motifs. (May et al., 2002). Table 5 shows the comparison of the Orf31 substrate specificity code to substrate specificity codes of other aryl acid-activating domains involved in the biosynthesis of the following secondary metabolites: virginiamycin (VisB, accession number BAB83672), pristinamycin (SnbA, accession number CAA67140), mycobactin (MbtA, accession number CAB03759), yersiniabactin (YbtE, accession number AAC69591), pyochelin (PchD, accession number AAD55799), neocarzinostatin (NcsB2, accession number AAM77987), vibriobactin (VlbE, accession number O07899), vulnibactin (Vva1301, accession number BAC97327), bacillibactin (DhbE, accession number AAC44632), and myxochelin (MxcE, accession number AF299336). Positions are numbered according to the GrsA phenylalanine-activating adenylation domain (Stachelhaus et al., 1999). Residues proposed to be involved in discrimination between the activation of 2′,3′-dihydroxybenzoic acid (DHBA) and salicylic acid are identified with an asterisk. Residues at each position matching that found in Orf31 are shaded in grey. HPA, 3-hydroxypicolinic acid.

Comparison of the Orf31 substrate specificity code to the codes of other aryl acid-activating enzymes and two enzymes that activate 3-hydroxypicolinic acid indicates that Orf31 activates either salicylic acid or HDBA. (Table 5).

TABLE 5
Comparison of aryl acid-activating domain substrate specificity codes

| | Amino Acid Position (GrsA numbering) | | | | | | | | | | |
|---|---|---|---|---|---|---|---|---|---|---|---|
| | 235 | 236 | 239 * | 278 | 299 | 301 | 322 | 330 * | 331 | 517 | Substrate |
| Virginiamycin | N | F | C | S | Q | G | V | L | T | K | HPA |
| Pristinamycin | N | F | C | S | Q | G | V | L | T | K | HPA |
| Mycobactin | N | F | C | A | Q | G | V | L | N | K | Salicylic |
| | | | | | | | | | | | acid |
| Yersiniabactin | N | F | C | A | Q | G | V | L | C | K | Salicylic |
| | | | | | | | | | | | acid |
| Pyochelin | N | F | C | A | Q | G | V | I | C | K | Salicylic |
| | | | | | | | | | | | acid |
| Neocarzinostatin | G | F | G | S | Q | G | V | L | C | K | Naphthoic |
| | | | | | | | | | | | acid |
| Orf31 | N | F | S | S | H | G | V | I | C | K | HDBA |
| Vibriobactin | N | F | S | A | Q | G | V | V | N | K | DHBA |
| Vulnibactin | N | F | S | A | Q | G | V | V | N | K | DHBA |
| Bacillibactin | N | Y | S | A | Q | G | V | V | N | K | DHBA |
| Myxochelin | N | F | S | A | Q | G | V | V | N | K | DHBA |

After activation of salicylic acid or HDBA, Orf31 catalyzes the transfer of the activated aryl acid to the sulfhydryl group of the phosphopantetheinyl prosthetic group attached to the ArCP, encoded by orf16. Orf16 is a small protein (95 aa), which is similar to many PCP and ArCP involved in secondary metabolism (˜30-40% identical) and it possesses the characteristic 4′-phosphopantheine attachment motif, including the invariant serine residue (GTFFQLRGQSI; SEQ ID NO:176). After attachment to the ArCP, the salicylate derivative is ready for incorporation into the Actinomadura sp. 21G792 enediyne complex, as discussed below.

This synthetic pathway is not considered limiting but merely illustrative. Using this as a model, one of ordinary skill in the art can design numerous other synthetic schemes to produce the 2-hydroxy-3,6-dimethylbenzoic acid component of Actinomadura sp. 21G792 chromophore or a derivative of this component.

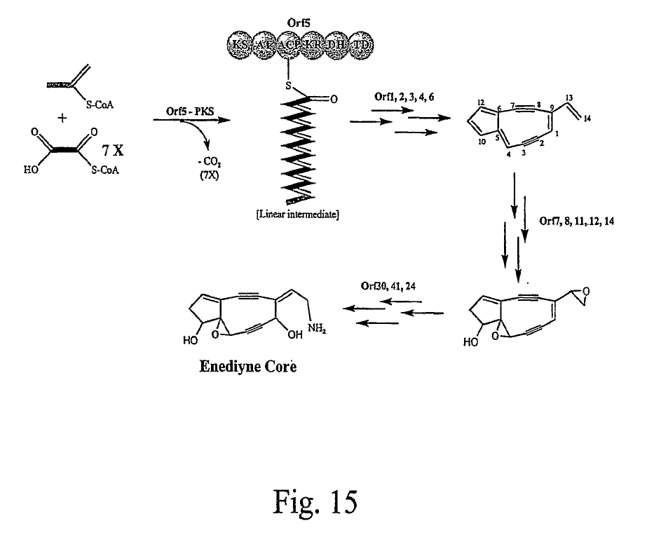

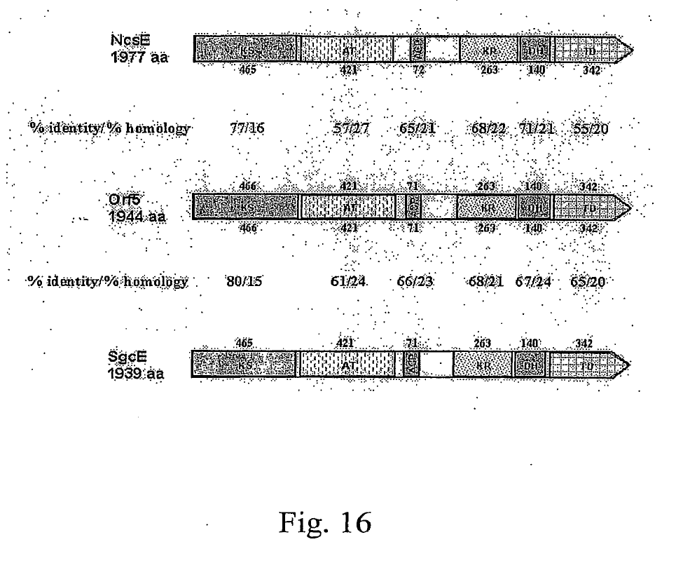

Enediyne core biosynthesis. At least fourteen genes were identified within the Actinomadura sp. 21G792 enediyne biosynthetic cluster whose deduced functions would support their roles in the Actinomadura sp. 21G792 enediyne core biosynthesis as outlined in FIG. 15. Orf5 encodes an iterative type I PKS that shows end-to-end sequence homology to the enediyne PKSs involved in the biosynthesis of neocarzinostatin (NcsE), C-1027 (SgcE) and calicheamicin (CalE8). (Liu et al., 2005; Liu et al., 2002; Ahlert et al., 2002, Science, 297, 1173-76). Like previously identified enediyne PKSs, Orf5 is composed of 6 domains: a KS, AT, ACP, KR, DH, and a so-called “terminal domain” (TD) (FIG. 16). The TD shows homology to 4′-phosphopantetheinyl transferases. Consequently, the TD has been proposed to catalyze the autoactivation of the enediyne PKS by post-translationally modifying the ACP active site serine with 4′-phosphopantetheine. (Zazopolous et al., 2003, Nature Biotech., 21, 187-90). Orf5 is expected to produce the nascent linear polyunsaturated polyketide intermediate from one acetyl-coA and 7 malonyl-coAs in an iterative fashion. The linear intermediate is possibly released from Orf5 and/or cyclized by Orf6, which shows similarity to a group of thioesterase proteins found in all enediyne biosynthetic clusters. Id. This group of proteins is predicted to function as thioesterases based on their homology to 4-hydroxybenzoyl-coA thioesterase of Pseudomonas sp. strain CBS-3. Id.

The polyketide intermediate is further processed by several gene products (Orfs 1-4, 7, 8, 11, 12, 14) to furnish the enediyne core (FIG. 15). These gene products are highly conserved in enedyine biosynthetic clusters. In addition to Orf5 and 6, homologs of Orfs 1-4 are found in all enediyne biosynthetic pathways studied to date (Id.), while homologs of Orfs 7, 8, 11, 12 and 14 are common to the 9-membered enediyne C-1027 and neocarzinostatin biosynthetic clusters. (Liu et al., 2005; Liu et al., 2002). Orfs 1-4, 11 and 14 are not homologous to any proteins of known function while Orfs 7, 8 and 12 resemble various oxidoreductases. Interestingly, it is possible that the expression of most of these genes is co-regulated, as orfs2-8 appear to be translationally coupled (e.g. the stop codon of orf2 overlaps the start codon of orf3, and the stop codon of orf3 overlaps the start codon or orf4, etc.) as are orf11 and orf12.

The enediyne core (FIG. 15) is further modified by a minimum of three gene products, Orf30, Orf41 and Orf24, which are likely involved in producing a terminal amide from the C13-C14 epoxide of the enediyne core. orf30 encodes a probable epoxide hydrolase, orf41 encodes an alcohol dehydrogenase and orf24 encodes an aminotransferase. The fully modified enediyne core moiety is subsequently adorned with the other chromophore components to produce the active metabolite.

This synthetic pathway is not considered limiting but merely illustrative. Using this as a model, one of ordinary skill in the art can design numerous other synthetic schemes to produce the endiyne core of the Actinomadura sp. 21G792 chromophore or a derivative of this component.

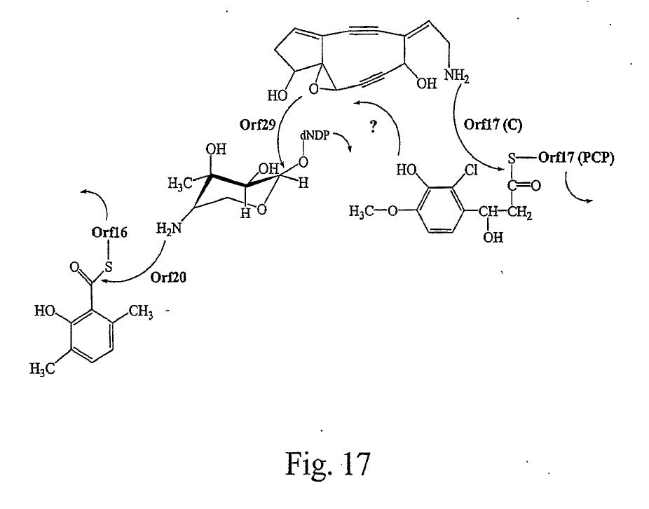

Assembly of the Actinomadura sp. 21G792 chromophore (FIG. 17). The biosynthesis of Actinomadura sp. 21G792 enediyne follows the current paradigm for enediyne biosynthesis, which calls for a convergent strategy for the assembly of the individual components of the molecular complex. (Liu et al., 2005; Liu et al., 2002; Ahlert et al., 2002). Following production of each component, they are systematically attached to the enediyne core to eventually furnish the final molecule as outlined in FIG. 17. The attachment of the enediyne core to the 3-(2-chloro-3-hydroxy-4-methoxy-phenyl)-3-hydroxy-propanyl-moiety is likely catalyzed by the condensation domain of Orf17. The catalysis of this reaction by Orf17 is consistent with the general peptide bond-forming activity normally attributed to the condensation domains of NRPSs. The mechanism used to attach the aromatic ring of the 3-(2-chloro-3-hydroxy-4-methoxy-phenyl)-3-hydroxy-propanyl-moiety to the enediyne core via ether bond formation is not known, however, it may occur concurrently with the opening of the C5-C6 epoxide and/or involve one or more of the P450 or monooxygenase encoding orfs contained within the Actinomadura sp. 21G792 enediyne biosynthetic cluster. The madurosamine moiety is coupled to the enediyne core via an β-glycosidic linkage. The gene product of orf29, which shows strong sequence similarity to a wide variety of glycosyltransferases involved in natural product biosynthesis, catalyzes this transfer. Orf29 is most similar to SgcA6 from the C-1027 biosynthetic pathway (43% identity, 57% similarity), which is proposed to catalyze the glycosylation of the C-1027 enediyne core. (Liu et al., 2002). Finally, Orf20, a type I NRPS condensation domain, transfers the HDBA-moiety from the phosphopatetheine arm of Orf16 to the amino group of madurosamine, in a reaction analogous to peptide bond formation in nonribosomal peptide biosynthesis.

Using this as a model, one of ordinary skill in the art can design numerous other synthetic schemes to produce the Actinomadura sp. 21G792 chromophores, particularly chromophore-b and chromophore-c, as well as derivatives of the chromophores.

The invention provides novel biosynthetic pathways comprising biosynthetic components of the Actinomadura sp. 21G792 chromophore, wherein one or more components has been mutated, or substituted or supplemented with a component from a biosynthetic pathway of a different enediyne chromophore, such that a variant of the Actinomadura sp. 21G792 chromophore is produced. Using standard molecular genetic techniques, individual orfs or combinations of orfs, as provided above, can be manipulated to produce novel bioactive analogs of the Actinomadura sp. 21G792 chromophore and/or chromoprotein. In one preferred embodiment, a novel chromophore is coexpressed with the Actinomadura sp. 21G792 apoprotein. In another embodiment, the Actinomadura sp. 21G792 chromophore is coexpressed with a variant of the Actinomadura sp. 21G792 apoprotein. In yet another embodiment, a novel chromophore is coexpressed with a variant of the Actinomadura sp. 21G792 apoprotein.

In an embodiment of the invention, inactivation of orf15 in Actinomadura sp. 21G792 produces an analog lacking the O-methyl that is usually found on the □-tyrosinyl moiety of the molecule. (See, e.g., FIG. 10) This change leaves a hydroxyl group in place of an O-methyl (see R¹ below). One reason for providing the hydroxyl group substitution would be to use it as a chemical handle for the further chemical derivitization of the analog by standard synthetic chemistry techniques. Similarly, inactivation of the halogenase encoded by orf19 prevents chlorination of PCP bound β-tyrosine, with the result that Cl is absent from the Actinomadura sp. 21G79 analog (see R² below). The R³ group indicated below is normally CH₃ and can be changed to H by inactivation the product of orf40 which methylates the 3-carbon of dNDP-L-xylose.

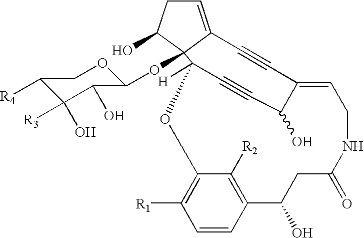

The R⁴ group of the Actinomadura sp. 21G792 chromophore is

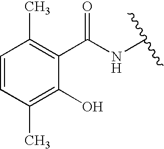

(designated R⁵), where R⁵ is linked to the sugar moiety at the amide nitrogen. Inactivation of orf32, causing production of an enediyne analog lacking the HDBA moiety (see, e.g., FIGS. 13, 17), or inactivation of orf20 results in substitution of R⁵ by NH₂. Further, the R⁴ moiety may be modified. For example,

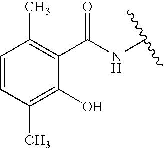

(designated R⁶) is obtained by inactivating orf33.

In another embodiment, orf32 is inactivated as above, and the mutant is used to produce a library of Actinomadura sp. 21G792 enediyne analogs where the HDBA moiety is replaced by other aryl acids. The aryl acids are introduced by feeding the orf32 mutant a variety of native aryl acids, N-acetyl cysteamine-linked

aryl acids, or aryl acids linked to other thioester carriers such as methyl thioglycolate in the fermentation broth. (See, e.g., Jacobsen et al. (1997) Science 277, 367-9). Each of the orfs involved in the addition of a component to the Actinomadura sp. 21G792 molecular complex can be mutated singly and in combination with other orfs to produce a large library of Actinomadura sp. 21G792 enediyne analogs for biological testing.

Thus, the invention provides compounds having the formula:

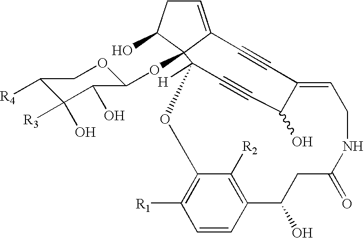

wherein R¹ is OH or OCH₃; R² is Cl or H; R³ is CH₃ or H; and R⁴ is selected from NH₂, R⁵, and R⁶. Further, by culturing an orf32 mutant in fermentation broth supplemented with particular native aryl acids, N-acetyl cysteamine-linked aryl acids, or aryl acids linked to other thioester carriers such as methyl thioglycolate, enediyne analogs can be produced wherein R⁴ is

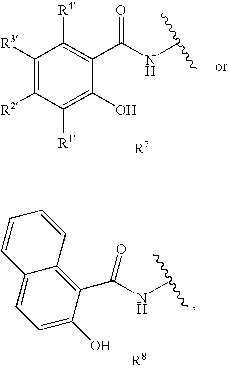

wherein R¹′ is H, CH₃, OH, OCH₃, Cl, C₃H₇, or NO₂; R²′ is H, CH₂, NH₂, OH, F, OCH₃, F, Cl, NO₂, OC₂H₅, or NC₂H₆; R³′ is H, CH₃, Cl, CH₃, NH₂, OH, F, COH, OCH₃, C₁, OC₂H₅, or NO₂; and R⁴′ is OH or OCH₃.

In other embodiments, one or more orfs from different secondary metabolic pathways can be introduced into Actinomadura sp. 21G792. Selected orfs can be introduced into the host chromosome by homologous recombination or by site specific integration mediated, for example, by a phage int/attP functionality (e.g. pSET152 or a similar vector). Alternatively selected orfs can be introduced on a self replicating vector. Once expressed, the gene products can proceed to modify the Actinomadura sp. 21G792 chromophore. For example, sgcA, sgcA1, sgcA2, sgcA3, sgcA4, sgcA5 and sgcA6 from the C-1027 biosynthetic gene cluster could be introduced into an Actinomadura sp. 21G792 strain in which one or more of the madurosamine biosynthetic orfs had been inactivated, in order to produce an Actinomadura sp. 21G792 enediyne analog comprising the C-1027 deoxy aminosugar, or a derivative thereof, in place of madurosamine.

The invention also provides for the introduction of genes from the chromoprotein biosynthetic cluster of Actinomadura sp. 21G792 into other secondary metabolite-producing microorganisms to modify the cognate secondary metabolite produced by that organism. For example, an analog of a different enediyne chromophore (e.g., the C-1027 chromophore) is produced by providing a host that expresses the biosynthetic pathway for that chromophore, and into which one or more of the components has been substituted or supplemented from the chromoprotein biosynthetic pathway of Actinomadura sp. 21G792.

In addition to making analogs of the Actinomadura sp. 21G792 chromoprotein, one can also increase fermentation titers by inactivating negative regulators as well as by increasing the expression level or gene copy number of positive regulators. The Actinomadura sp. 21G792 biosynthetic cluster contains at least eight orfs (orfs 9, 10, 46, 50, 52, 55, 62 and 63) identified as putative transcriptional regulators based on homology to sequences contained in the GenBank database. The function of these regulators can be tested in a systematic fashion to identify which regulator are positive regulators and which are negative regulators. Based on the findings, one could rationally alter one or more of these genes to increase fermentation titers of the Actinomadura sp. 21G792 chromoprotein.

Typically, organisms that produce toxic secondary metabolites possess one or more genes that confer self-resistance to the producing organism. The products of these genes usually confer resistance by chemically modifying, sequestering or transporting the toxic metabolite. In some cases, the target of the metabolite is innately insensitive to the metabolite, or the target is modified to confer insensitivity to the metabolite. The Actinomadura sp. 21G792 biosynthetic cluster contains at least two orfs whose gene products are likely involved in self-resistance. orf23, which encodes the apoprotein component of the Actinomadura sp. 21G792 complex, is presumably involved in sequestering the active chromophore, thereby shielding the DNA of the producing organism from cleavage by the chromophore. The gene product of orf22, encodes a protein similar to many transmembrane efflux proteins, and is most similar to SgcB from the C-1027 biosynthetic pathway, which has been proposed to act as an efflux pump for the C-1027 chromophore-apoprotein complex (Liu et al. (2005) Chem. Biol., 293-302). Using orf22 and orf23, one can potentially confer resistance to the Actinomadura sp. 21G792 chromoprotein. In one embodiment, these orfs can be introduced into a cell chosen to heterologously express the Actinomadura sp. 21G792 biosynthetic pathway, thereby allowing that cell to produce high levels of Actinomadura sp. 21G792 chromoprotein while being immune to its toxic effects. In another embodiment, these orfs can be introduced into donor cells chosen for biotransformation of Actinomadura sp. 21G792. Such cells would otherwise be killed by the extreme toxicity of Actinomadura sp. 21G792 before biotransformation could occur.

The entire Actinomadura sp. 21G792 biosynthetic cluster, or a selected portion, can be expressed in heterologous hosts such as bacteria. Examples of useful bacteria include, for example, members of the genera Streptomyces, Actinomadura, Nonomurea, Micromonospora, Escherichia, and Pseudomonas. (See, e.g., Pfeifer et al, 2001; Martinez et al., 2004) The biosynthetic cluster can also be heterologously expressed in a eukaryotic host such as yeast. In one embodiment, the Actinomadura sp. 21G792 biosynthetic cluster is advantageously expressed in an organism already modified for high level secondary metabolite production, thereby allowing for increased levels of Actinomadura sp. 21G792 chromoprotein production relative to that usually achieved using Actinomadura sp. 21G792. (See, e.g., Rodriguez et al., 2003, J. Ind. Microbiol. Biotechnol. 30, 480-8). In another embodiment, the Actinomadura sp. 21G792 biosynthetic cluster is advantageously expressed in an organism that is particularly amenable to genetic manipulation in order to expedite the generation of Actinomadura sp. 21G792 chromoprotein analogs (See, e.g., Bentley et al., 2002, Nature 417, 141-7; Binnie et al., 1997, Trends Biotechnol. 15, 315-20).

Various methods are known in the art that are useful for transferring recombinant DNAs encoding all or part of the Actinomadura sp. 21G792 chromoprotein biosynthetic pathway. Broad host-range plasmids are available that can be used to transfer and express such DNAs in a variety of hosts (e.g., pIJ101 for Streptomyces (Kieser et al., 1982, Mol. Gen. Genet. 185:223-8), pJRD215 for Actinomyces (Yeung et al., 1994, J. Bacteriol. 176:4173-6)). Methods for transferring such vectors include conjugation, electroporation and protoplast transformation. Shuttle vectors capable of replication in Escherichia coli and conjugal transfer from E. coli to gram-positive bacterial species such as Streptomyces spp. can also be used. (See, e.g., Mazodier et al., 1989, J. Bacteriol. 171:3583-5; Kieser et al., 2000, Practical Streptomyces genetics. A laboratory manual. John Innes Foundation, Norwich, United Kingdom).

It may be desired to prepare pharmaceutical compositions comprising a chromoprotein, wherein the chromoprotein comprises a complex of an apoprotein of the present invention and a chromophore, preferably the chromophore produced by Actinomadura sp. 21G792. Preferably, the polypeptide is attached to the chromophore via a non-covalent bond. Generally, preparing pharmaceutical compositions will entail preparing a pharmaceutical composition that is essentially free of pyrogens, as well as any other impurities that could be harmful to humans or animals. It may also be desirable to employ appropriate buffers to render the complex stable and allow for uptake by target cells.

Aqueous compositions of the present invention include an effective amount of the chromoprotein, further dispersed in a pharmaceutically acceptable carrier or aqueous medium. Such compositions also are referred to as inocula. The phrases “pharmaceutically or pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the chromoproteins, its use in the therapeutic compositions is contemplated. Supplementary active ingredients, including antibacterial or anti-tumor agents, also may be incorporated into the compositions.

In an embodiment of the invention, a chromophore of the invention is taken up by a cell, for example, by pinocytosis. In another embodiment, the chromophore is modified so as to be targeted to a particular cell or cell type. In one such embodiment, a chromoprotein may be delivered to target tissues in the form of polymers or conjugates employing monoclonal antibodies or other proteinaceous carriers as the targeting unit. Various polymer-based and antibody conjugate delivery systems are known and are currently being utilized in chemotherapeutic strategies involving the naturally-occurring C-1027 enediyne. In the present invention, the chromoproteins may, for example, be chemically-modified to form poly(styrene-co-maleic acid)-conjugated chromoproteins useful as therapeutics, particularly chemotherapeutics. (See, e.g., Maeda and Konno, 1997, in Neocarzinostatin: the Past, Present, and Future of an Anticancer Drug, H. Maeda, K. Edo, N. Ishida, Eds., Springer-Verlag, New York, pp. 227-267).

Polymeric micelles containing both hydrophobic and hydrophilic segments are new drug delivery systems recently developed to increase therapeutic indexes for chemotherapeutic agents (Yokoyama et al., 1990, Cancer Res. 50:1693-700; Kabanov et al., 1989, FEBS Lett. 258:343-5). Micelle size can be controlled so that the micelle particles are more permeable to blood vessels in tumor tissues than in normal tissues, owing to the enhanced permeability and retention (EPF) effect (Maeda, 2001, Adv Enzyme Regul. 41:189-207). This allows a favorable drug distribution in tumor tissues and hence the in vivo efficacy is expected to increase. The 21G792 chromoprotein can be non-covalently incorporated into specially designed micelles by mixing with a block copolymer solution. The metabolic stability of the resulting drug can be significantly increased (Yokoyama et al., 1991, Cancer Res. 51:3229-36), which potentially is advantageous for delivering 21G792 chromoprotein in cancer chemotherapy.

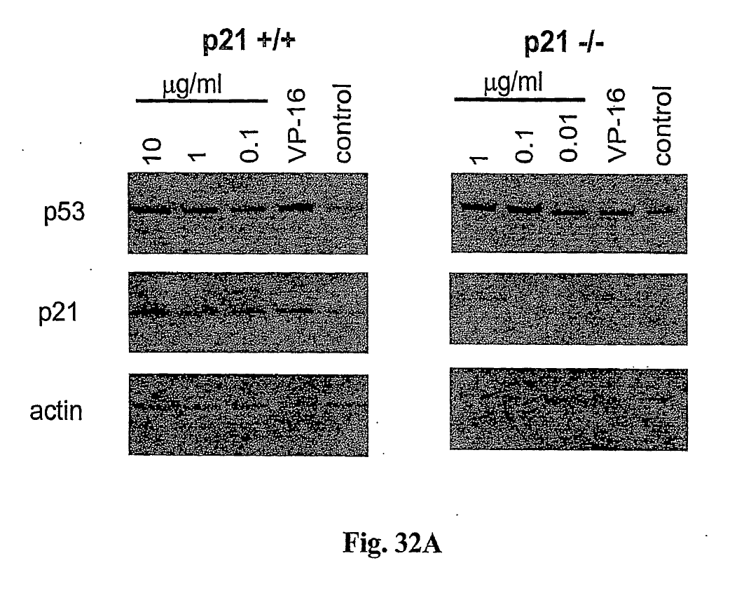

The chromoprotein (i.e., the apoprotein or chromophore) can be conjugated to a protein for delivery to a cell or a pathogen by the use of chemical linkers, or other related methods. The chromophore in the 21G792 chromoprotein has been reacted with sodium azide and secondary amines to give a series of derivatives. These derivatives contain an azide or secondary amino group at C-5 to replace the hydroxyl group in the natural chromophore. A linker with an amino group at one terminus and a carboxyl group at the other can be used to connect a monoclonal antibody and the chromophore to form a chromophore-antibody conjugate for targeted drug delivery. The amino group of the linker that is to replace the C-5 hydroxyl group is designed so that the conjugate can be hydrolyzed back to the chromophore under the more acidic condition in tumor tissues. An exemplary linkage is depicted in FIG. 30.

In addition, the chromoproteins may be conjugated with monoclonal antibodies to form monoclonal antibody (MAb)-chromoprotein conjugates. Antibodies with high affinity for antigens, preferably having specificity for antigenic determinants on the surface of malignant cells, are a natural choice as targeting moieties. Antibody-mediated specific delivery of the chromoproteins to tumor cells is expected to not only augment their anti-tumor efficacy, but also prevent nontargeted uptake by normal tissues, thus increasing their therapeutic indices. Examples of such antibody carriers that may be used in the present invention include monoclonal antibodies, chimeric antibodies, humanized antibodies, human antibodies, biologically active fragments thereof and their genetically or enzymatically engineered counterparts. Preferably, such antibodies are directed against cell surface antigens expressed on target cells and/or tissues in proliferative disorders such as cancer. The anti-CD33 monoclonal antibody is illustrative of a useful Mab for this approach and may effectuate the targeting of a chromoprotein to cancerous tissues in various contexts, including in patients afflicted with acute myeloid leukemia. (See, e.g., Sievers et al., 1999, Blood 93, 3678-84) Another example of a useful monoclonal antibody conjugate is described in PCT Publication No. WO 03/029623 in which, for example, an anti-CD22 monoclonal protein is conjugated to an enediyne for targeted delivery to B-cell lymphomas. As previously noted, several MAb-C-1027 conjugates are under evaluation as promising anticancer drugs. (Brukner, 2000, Curr. Opinion Oncologic, Endocrine & Met. Invest. Drugs 2, 344). Other proteinaceous carriers in addition to antibody carriers include hormones, growth factors, antibody mimics, and their genetically or enzymatically engineered counterparts, hereinafter referred to singularly or as a group as “carriers.” The essential property of a carrier is its ability to recognize and bind to an antigen or receptor associated with undesired cells and to be subsequently internalized. Examples of carriers that are applicable in the present invention are disclosed in U.S. Pat. Nos. 5,053,394 and 5,773,001, which are incorporated herein in their entirety. Preferred carriers for use in the present invention are antibodies and antibody mimics.

A number of non-immunoglobulin protein scaffolds have been used for generating antibody mimics that bind to antigenic epitopes with the specificity of an antibody (PCT publication No. WO 00/34784). For example, a “minibody” scaffold, which is related to the immunoglobulin fold, has been designed by deleting three beta strands from a heavy chain variable domain of a monoclonal antibody (Tramontano et al., 1994, J. Mol. Recognit. 7:9-24). This protein includes 61 residues and can be used to present two hypervariable loops. These two loops have been randomized and products selected for antigen binding, but thus far the framework appears to have somewhat limited utility due to solubility problems. Another framework used to display loops is tendamistat, a protein that specifically inhibits mammalian alpha-amylases and is a 74 residue, six-strand beta-sheet sandwich held together by two disulfide bonds, (McConnell and Hoess, 1995, J. Mol. Biol. 250:460-70). This scaffold includes three loops, but, to date, only two of these loops have been examined for randomization potential.

Other proteins have been tested as frameworks and have been used to display randomized residues on alpha helical surfaces (Nord et al., 1997, Nat. Biotechnol. 15, 772-7; Nord et al., 1995, Protein Eng. 8, 601-8), loops between alpha helices in alpha helix bundles (Ku and Schultz, 1995, Proc. Natl. Acad. Sci. USA 92, 6552-6), and loops constrained by disulfide bridges, such as those of the small protease inhibitors (Markland et al., 1996, Biochemistry 35, 8045-57; Markland et al., 1996, Biochemistry 35, 8058-67; Rottgen and Collins, 1995, Gene 164, 243-50; Wang et al., 1995, J. Biol. Chem. 270, 12250-6).

The targeting molecule and chromoprotein may be covalently associated by chemical cross-linking or through genetic fusion such as by application of recombinant DNA techniques. In the latter approach, the apoprotein may be fused at its C-terminus or N-terminus to the N-terminus or C-terminus of the cell targeting protein molecule. When the cell targeting molecule is an antibody, the N-terminus of the apoprotein is preferably fused to the C-terminus of the light and/or heavy chain of the antibody. For chemical cross-linking, some common protein-antibody linkers are succinate esters and other dicarboxylic acids, glutaraldehyde and other dialdehydes. Other such linkers are well known in the art.

Solutions of therapeutic compositions may be prepared in water suitably mixed with a surfactant (e.g., hydroxypropylcellulose). Dispersions also may be prepared in glycerol, liquid polyethylene glycols, mixtures thereof, and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical administration. Topical administration would be particularly advantageous for treatment of skin cancers, to prevent chemotherapy-induced alopecia or other dermal hyperproliferative disorder. Alternatively, administration will be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, the preferred route is aerosol delivery to the lung. Volume of the aerosol is between about 0.01 ml and 0.5 ml. Similarly, a preferred method for treatment of colon-associated disease would be via enema. Volume of the enema is between about 1 ml and 100 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance.

Throughout this application, various publications are referenced in parentheses by name or number. The disclosures of these publications in their entireties are hereby incorporated by reference into this application to more fully describe the state of the art to which this invention pertains.

### EXAMPLES

It is to be understood and expected that variations in the principles of the invention herein disclosed may be made by one skilled in the art and it is intended that such modifications are to be included within the scope of the present invention.

Examples of the invention which follow are set forth to further illustrate the invention and should not be construed to limit the invention in any way.

### Example 1

### Isolation and Characterization of the Chromoprotein and Apoprotein

### Example 1-Seed Culture

Actinomadura sp. 21G792 was preserved as frozen whole cells (frozen vegetative mycelia, FVM) prepared from cells grown for 72 hours in ATCC medium 172 (Dextrose 1%, Soluble Starch 2%, Yeast Extract 0.5%, and N-Z Amine Type A 0.5%, CaCO₃ 0.1% pH 7.3). Glycerol was added to 20% and the cells were frozen at −150° C.

A seed medium having a pH of 6.9 was prepared containing: 1.0% dextrose; 2.0% soluble starch; 0.5% yeast extract; 0.5% N-Z Amine Type A (Sheffield); and 0.1% CaCO₃. In a 25 mm×150 mm glass culture tube, 7 ml of the seed medium and two glass beads were inoculated with cells of Actinomadura sp. 21G792 cultured on ATCC agar medium #172 (ATCC Media Handbook, 1^{st} edition, 1984). Sufficient inoculum from the agar culture was used to provide a turbid seed after 72 hours of growth. The primary seed tubes were incubated at 28° C., 250 rpm using a gyro-rotary shaker with a 2 inch throw, for 72 hours. The primary seed (˜14% inoculum) was then used to inoculate a 250 ml Erlenmeyer flask containing 50 ml of medium #172. These secondary seed flasks were incubated at 28° C., 250 rpm using a gyro-rotary shaker (2″ stroke), for 48 hours.

### Example 2

### Example 2-Fermentation

A fermentation production medium having a pH of 6.9 was prepared containing: 2.0% sucrose; 0.5% molasses; 0.5% CaCO₃; 0.2% peptone; 0.002% magnesium sulfate-7H₂O; 0.001% ferrous sulfate-7H₂O; 0.05% sodium bromide; and 0.2% sodium acetate. Sixty 250 ml Erlenmeyer flasks were each prepared with 50 ml of the fermentation production medium and inoculated with 2 ml (4.0%) of the secondary seed fermentation and incubated at 28° C. at 250 rpm using a gyro-rotary shaker (2″ stroke). The fermentation as described was then allowed to proceed for approximately 72 to 96 hours and harvested for further processing.

The combined whole broth (60×50 ml) was centrifuged at 3800 rpm for 30 minutes. The supernatant was then lyophilized and the residual powder was suspended in a small volume (e.g., 300 ml) of H₂O. Upon centrifugation, the brownish solution was then loaded onto a glass column containing 6 L of Sephadex G75 in H₂O at 4° C. in the dark. Fractions of 40 ml each were collected and tested in a biochemical induction assay (BIA). The most potent fractions were then combined (15 fractions, 600 ml total) and lyophilized. The grayish powder was then dissolved in H₂O (4 ml) and analyzed by HPLC to contain two major peaks, one corresponding to the apoprotein and the other corresponding to the chromoprotein.

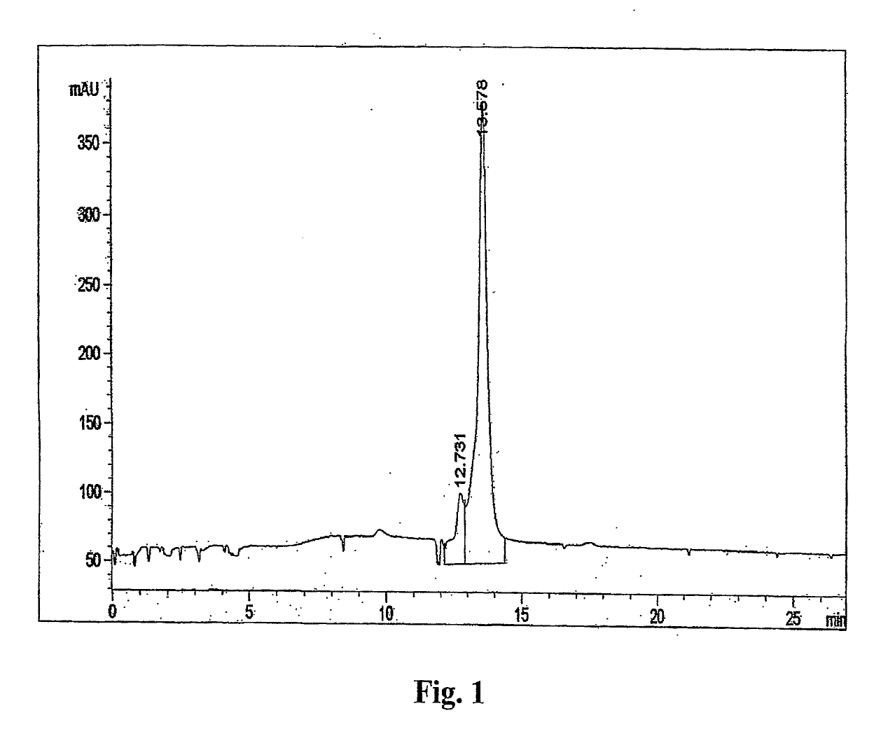

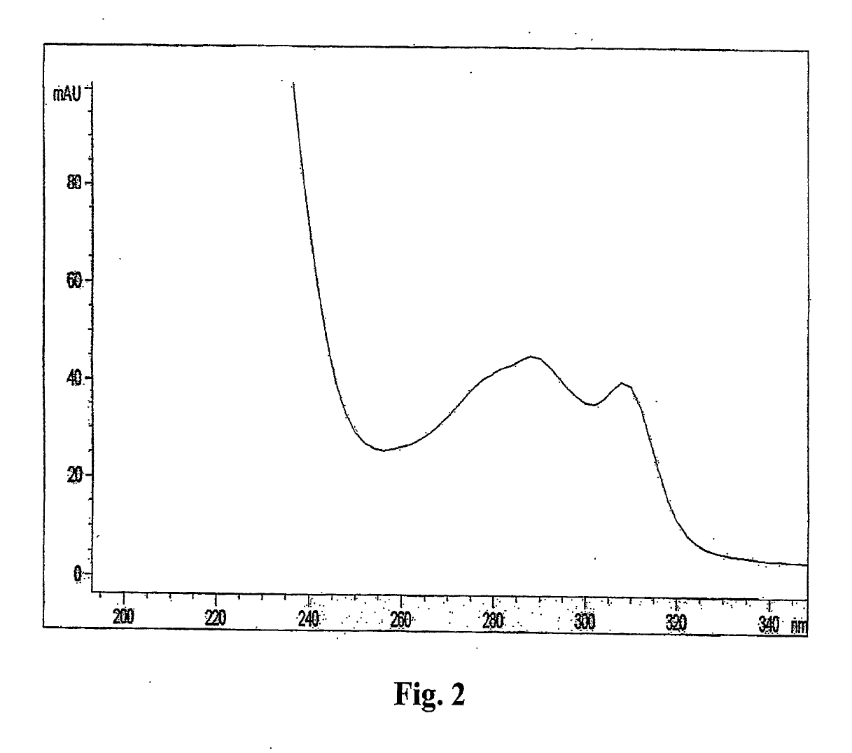

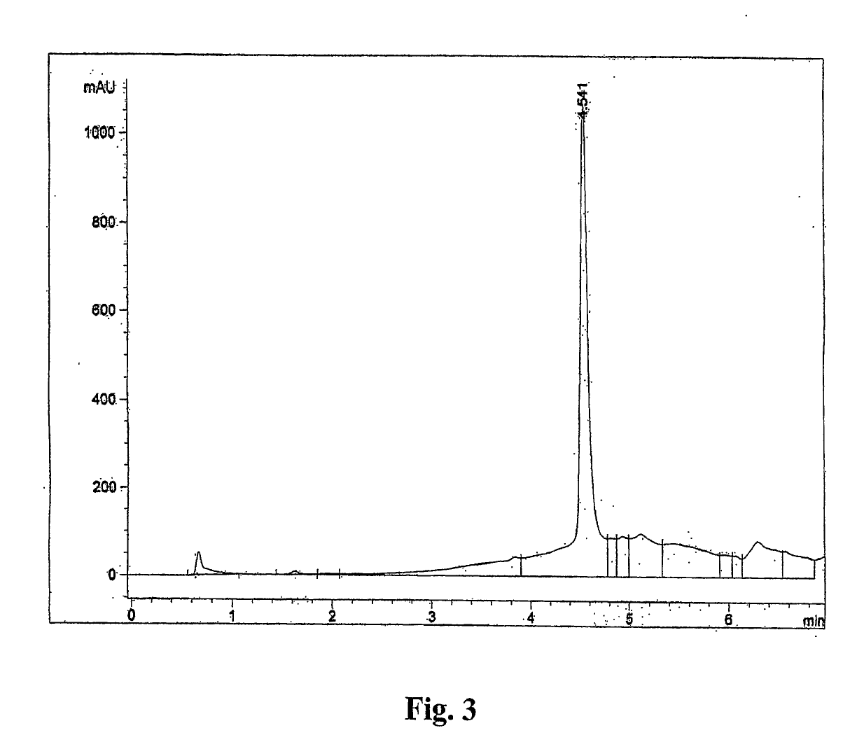

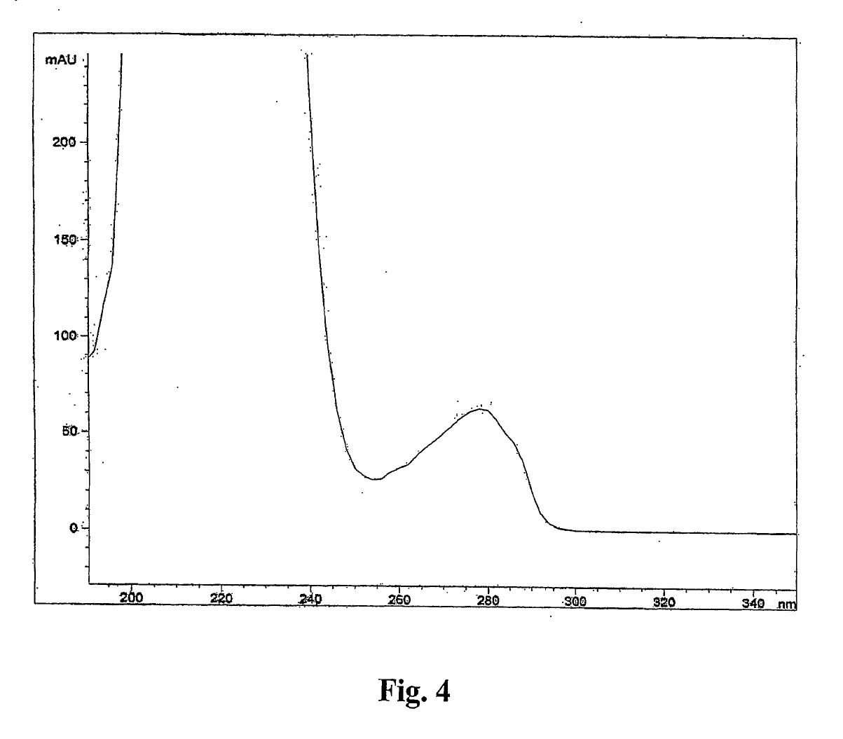

The above solution was subjected to preparative HPLC chromatography on a TosoHaas DEAE 5PW column (13 um particle size, 21.5 mm×15 cm in size) with a buffer system (0-0.5 M linear gradient NaCl with constant 0.05 M Tris-HCl in 30 min) at a flow rate of 4 ml/min. The respective peaks of apoprotein and chromoprotein were collected, desalted with Pierce Dialysis Cassette (7000 MWCO), and lyophilized. The resulting powders of apoprotein and chromoprotein were then repurified by the same preparative HPLC conditions, desalted, and lyophilized. The final products of chromoprotein (grayish powder, 10.5 mg) and apoprotein (white powder, 19.8 mg) were analyzed by analytical HPLC (FIGS. 1 and 3, respectively). The ultraviolet absorption (UV) spectra of the chromoprotein and apoprotein are shown in FIGS. 2 and 4.

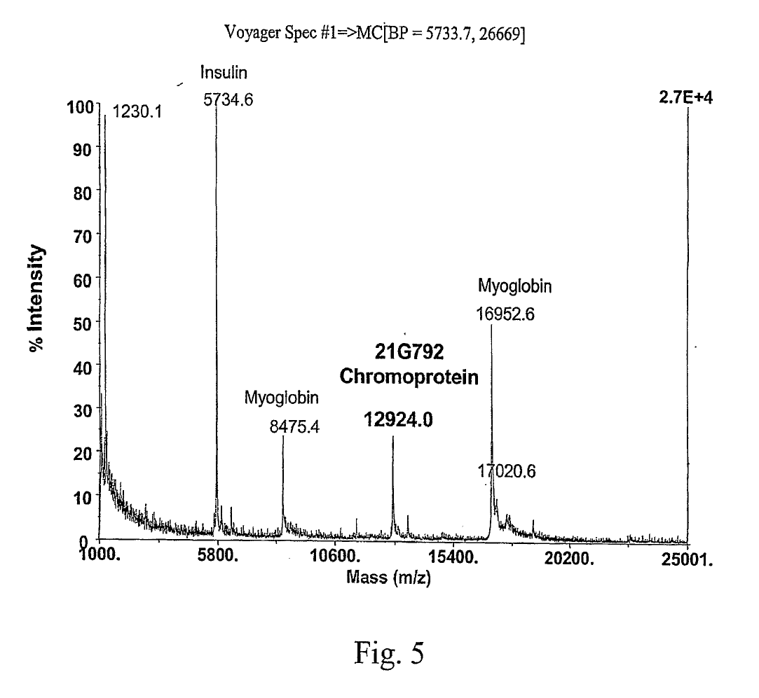

The molecular weight of the apoprotein was determined to be 12.92409 kDa by MALDI-MS. The MALDI spectrum is shown in FIG. 5.

### Example 3

### Example 3-Inoculum

1 liter of sterile inoculum medium was prepared for a pilot scale fermentation using the following components: 5.0 g/L Phytone (BBL), 5.0 g/L yeast extract (Bacto), 40 g/L soluble starch, 20 g/L glucose, 1.28 g/L magnesium sulfate heptahydrate, 0.025M MOPS. About 1 mL of seed culture was transferred to the sterila flask before it was placed on a shaker and incubated at 30° C. and 200 rpm. The entire contents of the flask were transferred to a fermenter aseptically after 48 to 72 hours of incubation.

### Example 4

### Example 4-Pilot Scale Fermentation

Chromoprotein fermentation was conducted in the 100 L pilot fermenter using Actinomadura sp. strain 21G792 as the producing microorganism. The following medium was prepared with purified water up to 65 L: glucose monohydrate, 2.75 g/L; ferrous sulfate heptahydrate, 0.01 g/L; magnesium sulfate heptahydrate, 0.02 g/L; calcium carbonate (Mississippi™ Lime), 2.0 g/L; Marcor martone J-1, 4.0 g/L; sodium acetate trihydrate, 2 g/L; potassium iodide, 0.5 g/L; Pluronic L-61, 0.5 g/L. The pH of the medium solution was adjusted to 7.0 with sulfuric acid. The medium was then sterilized and cooled down to the fermentation temperature before an additional amount of glucose monohydrate (about 6 g/L) and one liter of inoculum were transferred to the fermenter.

The fermentation temperature was controlled at a proper value (30° C.) to support the cell growth and production and carried out with proper agitation, pressure, and aeration to maintain the dissolved oxygen above 20% of the saturation value. In this example, the fermentation was controlled at 250 rpm, 5 psig, and 30 lpm.

The batch was harvested at day 4. The concentration of chromoprotein was 152 mg/L at harvest. The harvested mash was clarified by centrifugation in a centrifuge that accepts 1 L centrifuge bottles. The centrifugation conditions used were 7000 rpm, 20° C., and 20 minutes per cycle. The supernatant was collected for further product recovery.

### Example 5

### Example 5-Pilot Scale Fermentation

Chromoprotein fermentation using Actinomadura sp. strain 21G792 was performed in the following medium: glucose monohydrate, 8.75 g/L; ferrous sulfate heptahydrate, 0.01 g/L; magnesium sulfate heptahydrate, 0.02 g/L; calcium carbonate (Mississippi™ Lime), 2.0 g/L; Marcor martone J-1, 4.0 g/L; sodium acetate trihydrate, 2 g/L; sodium bromide, 0.5 g/L; Pluronic L-61, 0.5 g/L. The batch was harvested at 70 hours and produced about 60 mg/L of chromoprotein.

### Example 6

### Example 6-Chromoprotein Assay

This assay method separates the chromoprotein from UV absorbing components of fermentation broth and resolves the chromoprotein and apoprotein forms. The instrument is a Waters Alliance 2695 equipped with a 2996 PDA detector, Millennium chromatography control and analysis software. The column is a TSK Phenyl 5PW 10 μm diameter particle 7.5×75 mm supplied by TOSOH Biosciences or SUPELCO. Mobile phase is A: 1.5 M ammonium sulfate, 20 mM Tris pH 7.8-8.3, B: 20 mM Tris 100 mM NaCl pH 7.8-8.3. The runtime is 30 min at a flow rate of 1 mL/min. Detection is at 230 nm, with spectra collected over 200-400 nm with a 4.8 nm bandwidth. A gradient elution is programmed as: 0-3 min hold 100% A, 3-23 min linear to 100% B, 23-24 linear to 100% A, 24-30 hold 100% A. The injection volume is 100 μL or less. In-process samples are applied without further cleanup, other than 0.45 μm filtration. Chromoprotein and apoprotein peaks are identified by comparison to a reference standard retention time and spectra. Retention times of 18 min for chromoprotein and 20 min for apoprotein are typical. Quantitation of the integrated chromoprotein peak is performed as follows:

mg/mL chromoprotein=Dil factor*(component area μV*sec×7.55×10⁻⁶μg/μV*sec)/injection volume μl)

### Example 7

### Example 7-Chromoprotein Assay

This assay method separates the chromoprotein from other UV absorbing components of the fermentation broth and other contaminating proteins on the basis of molecular size. The chromoprotein and apoprotein forms coelute. The instrument is a Waters Alliance 2695 with a 2996 PDA detector and Millennium chromatography control and analysis software. The column is a BioSil SEC 125, 7.8×300 mm supplied by BioRad. Mobile phase is A: 20 mM Tris 100 mM NaCl pH 7.8-8.3. Detection is at 230 nm, with spectra collected over 200-400 nm with a 4.8 nm bandwidth. The runtime is 20 min at a flow rate of 1 mL/min. The elution is iosocratic. The injection volume is 100 ul or less. In-process samples are applied without further cleanup other than 0.45 μm filtration. The chromoprotein peak is identified by comparison to reference standard retention time and spectra. A retention time of 7.8 min for chromoprotein is typical. Quantitation of the integrated chromoprotein peak is performed as follows:

mg/mL chromoprotein=Dil factor*(component area μV*sec×7.55×10⁻⁶μg/μV*sec)/injection volumeμl)

### Example 8

### Example 8-Biochemical Induction Assay

The biochemical induction assay identifies DNA damaging agents which induce the SOS response in E. coli BR513-80. BR513-80 contains a translational fusion of the λ PL promoter and the lacZ gene. The λ PL promoter, activated as part of the SOS response after exposure to DNA damaging agents, drives the synthesis of β-galactosidase. β-galactosidase activity is detected by the cleavage of the 6-bromo-2-naphthyl-β-D-galactopyranoside (BNG) which reacts with Fast Blue RR salt to produce a red/purple color.

A basal layer of solid media is prepared using LBE (Bacto Tryptone 10 g/L, Yeast Extract 5 g/L, NaCl 10 g/L, 1M Tris Base 5 mL/L), or equivalent, containing 15 g/L agar, 0.2% glucose, and “E” solution diluted 1/125. (“E” solution contains 5 g magnesium sulfate heptahydrate, 50 g citric acid monohydrate, 250 g potassium phosphate dibasic, and 87.5 g sodium ammonium phosphate diluted in order into 1 L deionized H₂O.) About 170 ml of the basal layer medium is used per Nunc BioAssay plate, or about 50 mL per 150 mm petri dish.

A BR513-80 overnight culture is diluted 1:20 in LBE broth or equivalent, and the absorbance of the diluted culture is measured at 600 nm. The absorbance of the undiluted culture is calculated, and 5.7 is divided by this value to obtain the volume (in mL) of culture to use as an inoculum per 40 mL soft agar. Typically this volume is about 0.9-1.3 mL. The calculated culture volume is swirled in soft agar, poured evenly over the base layer (40 ml per Nunc BioAssay plate, or 20 mL per 150 mm petri dish), and allowed to solidify for at least 10-15 minutes.

Samples and standards to be assayed are applied in 10 μL aliquots directly to the surface of the soft agar overlay. Useful standards include bleomycin at 10, 5, 2.5, and 0.31 μg/mL, and calicheamicin γ′1 at 1000, 100, 10, 1 and 0.1 ng/mL. The assay plates are incubated at about 37° C. for approximately 3 h to induce the SOS response.

A dye/substrate overlay is prepared by dissolving 87 mg Fast Blue RR salt and 13 mg BNG in 2 mL DMSO and mixing with 40 mL melted 1% soft agar. After the incubation period, 40 mL of the dye/substrate mixture (20 mL for the 150 mm dish) is evenly poured over the surface of the BIA plate, and allowed to solidify. The induction response is scored after 15-20 minutes at room temperature or after 10 min. at about 37° C.

The following exemplifies a scoring scale: 4+ circular 20-25 mm red/purple induction zone with or without a clear central area; 3+ circular 15-20 mm red/purple induction zone with or without a clear central area; 2+ circular 10-15 mm red/purple induction zone with or without a clear central area; 1+ faint red zone with or without a clear central area; +/−small diffuse red zone with or without a clear central area; T Toxic zone (clear area where bacteria have been killed and no color change); N/R No red/purple induction zone; no clear toxic zone

### Example 9

### Example 9-Chromoprotein Purification

This example provides a scaleable purification process for the isolation of approximately 90% pure, BIA active chromoprotein. The compound isolated by this process was analyzed by SDS-gel electrophoretic mobility, UV spectra and HPLC retention times using both size exclusion and hydrophobic interaction analytical columns. The fermentation conditions were not optimized for chromoprotein production.

Chromoprotein from 350 mL of a clarified fermentation broth was purified using a scaleable 3-step procedure consisting of DEAE Sepharose FF ion exchange chromatography, Phenyl Sepharose HP hydrophobic interaction chromatography and Superdex 75 size exclusion chromatography. Step yields of about 61, 69 and 82% respectively, result in 30% process recovery overall of chromoprotein that is about 90% pure, with the balance of the material being the inactive apoprotein.

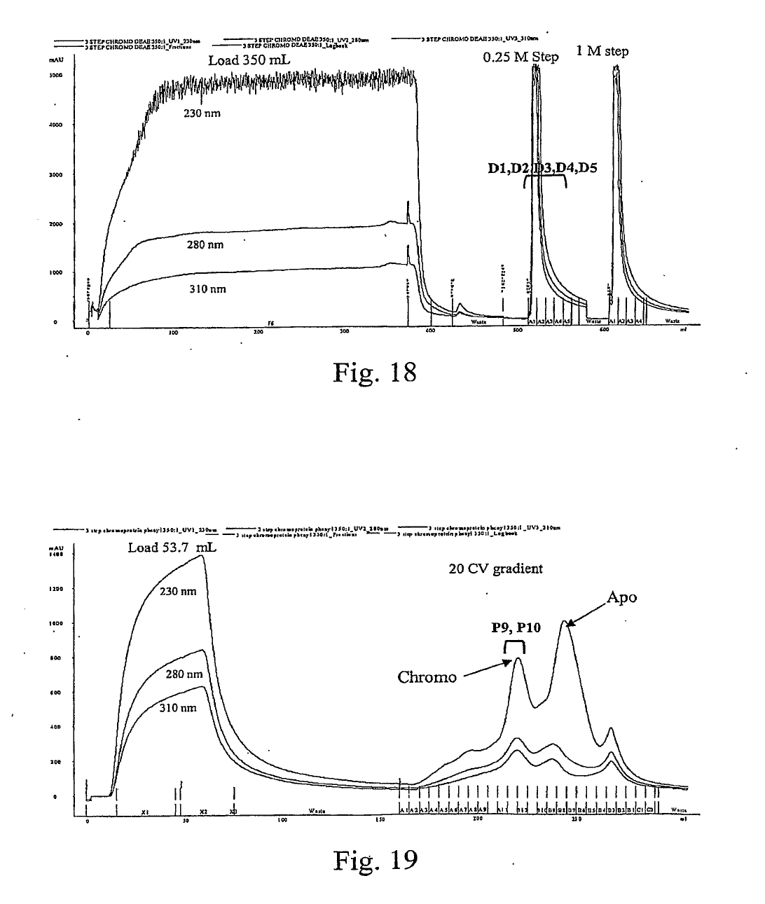

DEAE Sepharose FF anion exchange chromatography—The chromatography load was prepared by the addition of 7 mL of 1 M Tris pH 8.3 to 350 mL of clarified broth. The load was then applied to a 5 mL HiTrap DEAE Sepharose FF column equilibrated with 5 column volumes (CV) of 20 mM Tris pH 8.3 at a flow rate of 10 mL/min. Absorbance was monitored at 230, 280 and 310 nm. The effluent of the column was collected as a single fraction. The column was washed with 20 mM Tris pH 8.3 until the absorbance reached baseline. The chromoprotein species were eluted by stepping the mobile phase to 0.25 M NaCl, 20 mM Tris pH 8.3 in a 10 CV fraction (collected as 5 fractions of 2 CV; see Table 6, fractions D1-D5). A second elution step was performed using 1 M NaCl, 20 mM Tris pH 8.3 to wash the column. FIG. 18 shows the elution profile.

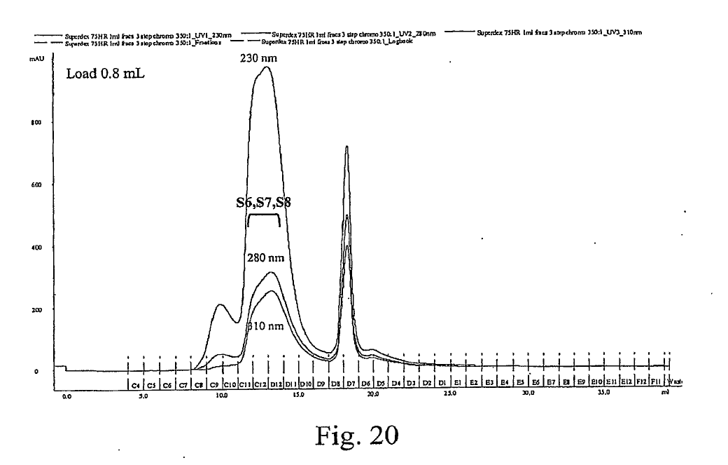

Phenyl Sepharose HP Chromatography—The chromatography load was prepared by adding 10.6 g of ammonium sulfate to 47.5 mL of the saved eluate from the first step to bring the ammonium sulfate concentration to 1.5 M. The volume of the load (after ammonium sulfate addition) was calculated to be 53.7 ml. The pH of the load was raised from 7.8 to 8.06 following the ammonium sulfate addition using 1 mL of 1 M Tris pH 8.3. The clear, brown solution was easily filtered through a 0.45 μm nylon syringe filter (2.3 cm dia.) and 52.7 mL applied to a 5 mL HiTrap Phenyl Sepharose HP column at 5 mL/min. The column had been previously equilibrated with 5 CV of 1.5M ammonium sulfate, 20 mM Tris pH 8.2. The absorbance was monitored at 230, 280 and 310 nm. Separation of the chromoprotein from the apoprotein occurs over a 20 CV linear gradient elution to 100 mM NaCl, 20 mM Tris pH 8.2. Single CV fractions were collected throughout the gradient and two fractions chosen as the chromoprotein peak by UV absorbance (denoted P9-P10 in Table 6) were pooled. The chromoprotein elutes prior to the apoprotein and the two peaks are separated by 2 CV. FIG. 19 shows the elution profile.

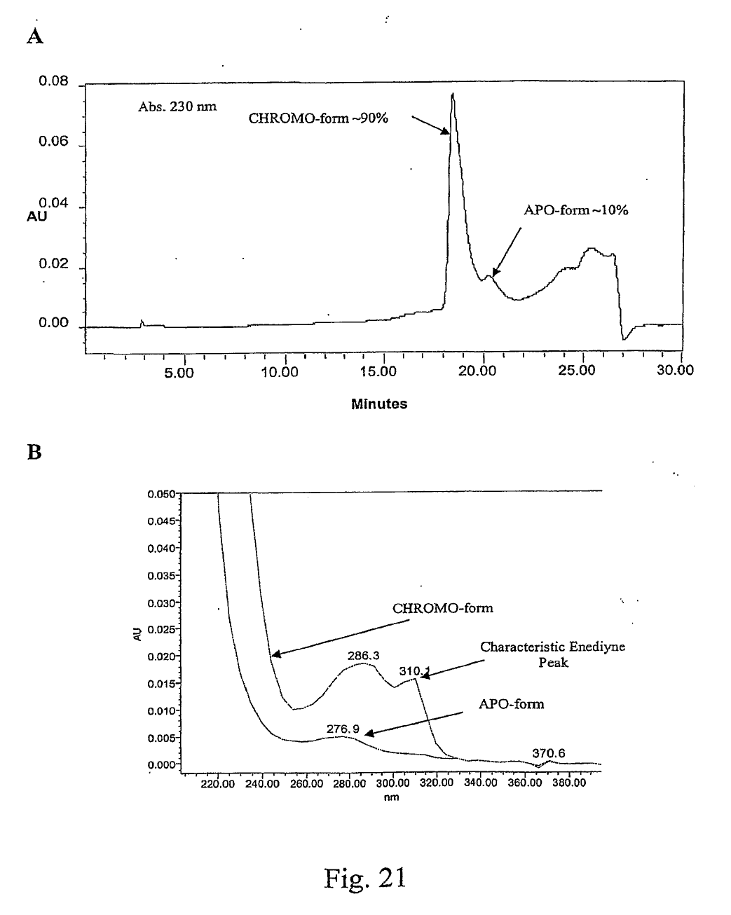

Superdex 75 Chromatography—Ammonium sulfate was added to 9.5 ml of the phenyl sepharose pool to a concentration of 80% (560 g solid/L of phenyl sepharose pool). The precipitate that formed following addition of the ammonium sulfate was captured on a 1 inch dia. 0.45 μm nylon syringe filter and recovered by repeated extraction of the solid with 1 mL of 20 mM Tris, 100 mM NaCl pH 8.2. A 0.8 mL portion of the extract was applied to a Superdex 75 column equilibrated in 2 CV of 40 mM Tris 200 mM NaCl and eluted over 1.5 CV with 1 mL fractions collected. The absorbance was monitored at 230, 280 and 310 nm at a flow rate of 0.5 mL/min. The chromoprotein pool (3 ml) was selected as the three fractions denoted S6-S8 in Table 6. FIG. 20 shows the elution profile.

TABLE 6
YIELDS OF CHROMOPROTEIN AND APOPROTEIN FROM
PILOT SCALE FERMENTATION BROTH.

| | Chromoprotein | Chromoprotein | Apoprotein |
|---|---|---|---|
| Sample | (mg/mL) | (mg) | (mg) |
| Broth (DEAE LOAD) | 0.037 | 13.20 | 26.40 |
| 350 mL | | | |
| D1 (pooled) | 0.848 | 8.40 | 25.80 |
| D2 (pooled) | 0.298 | 3.00 | 4.60 |
| D3 (pooled) | 0.014 | 0.14 | 0.14 |
| D4 (pooled) | 0 | 0 | 0 |
| D5 (pooled) | 0 | 0 | 0 |
| Phenyl HP LOAD | 0.151 | 8.10 | 25.90 |
| 52.7 mL | | | |
| P1-P7 | 0 | 0 | 0 |
| P8 | 0.073 | 0.36 | 0.22 |
| P9 (pooled) | 0.450 | 2.25 | 0.60 |
| P10 (pooled) | 0.680 | 3.40 | 0.32 |
| P11 | 0.340 | 1.70 | 0.11 |
| P12 | 0.090 | 0.45 | 1.39 |
| P13 | 0.151 | 0.76 | 2.07 |
| P14 | 0 | 0 | 7.21 |
| P15 | 0 | 0 | 7.85 |
| P16 | 0 | 0 | 4.83 |
| P17 | 0 | 0 | 1.58 |
| P18 | 0 | 0 | 0.09 |
| SEC LOAD 0.8 mL | 4.53 | 3.62 | 0.48 |
| S4 | 0 | 0 | 0 |
| S5 | 0.55 | 0.55 | 0.13 |
| S6 (to be pooled) | 1.36 | 1.36 | 0.17 |
| S7 (to be pooled) | 1.17 | 1.17 | 0.14 |
| S8 (to be pooled) | 0.43 | 0.43 | 0.07 |
| S9 | 0.04 | 0.04 | 0.01 |

TABLE 7
IN-PROCESS RECOVERIES OF THE CHROMOPROTEIN

| | |
|---|---|
| DEAE | 61% |
| PHENYL HP | 69% |
| SUPERDEX | 82% |
| OVERALL | 30.5% |

Recoveries are corrected for in-process sampling. Overall yield was calculated using a pool of Superdex fractions S6, S7 and S8.

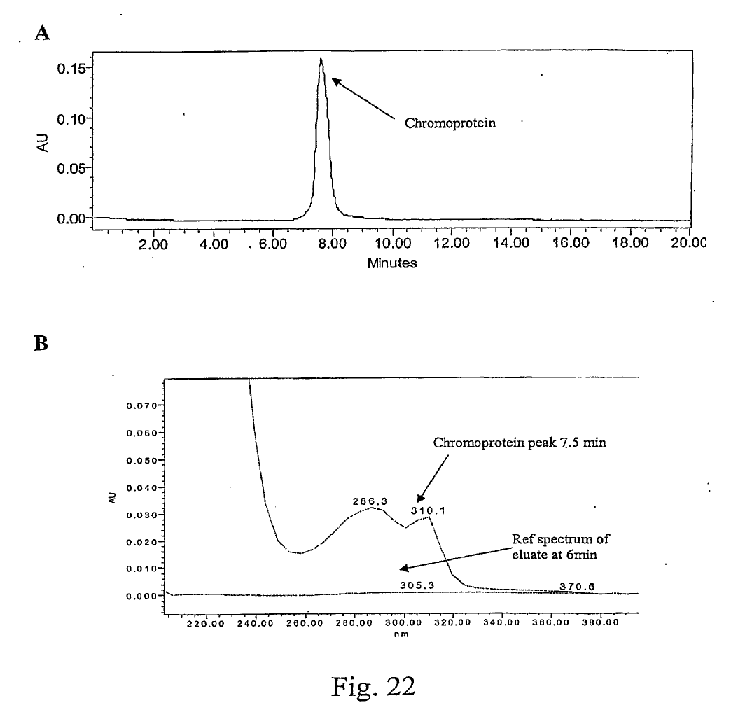

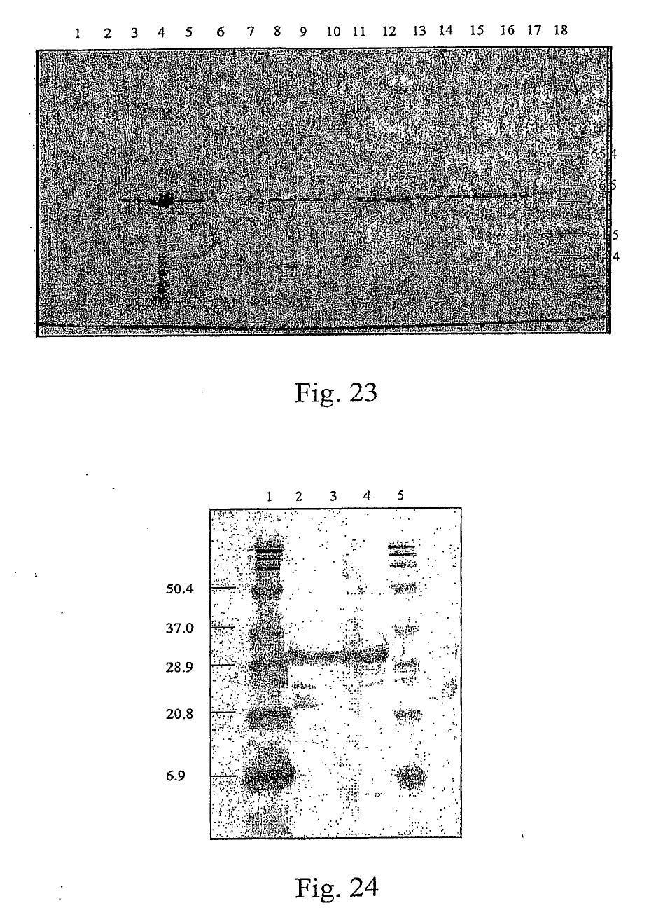

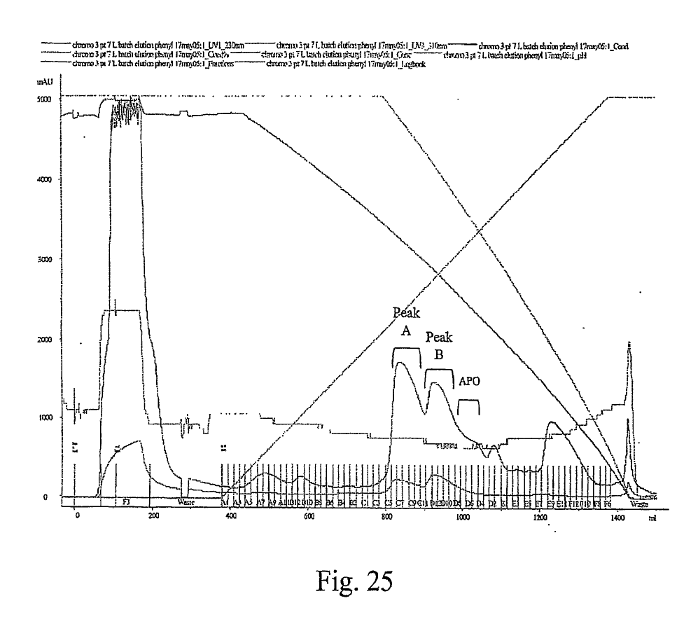

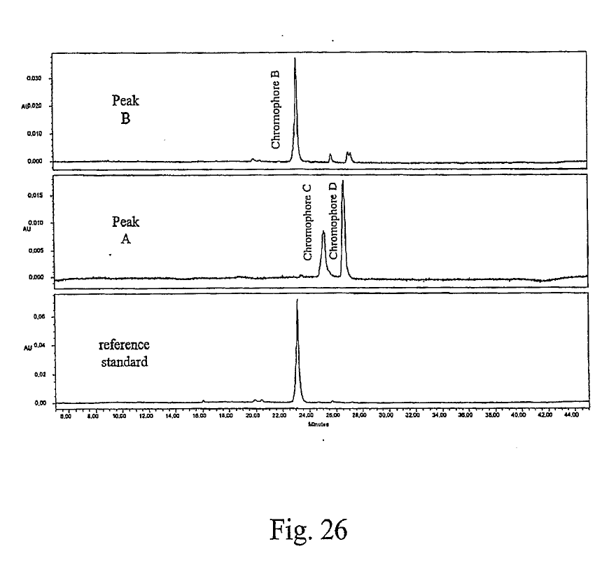

The amount of chromoprotein recovered in each purification step and for the overall process is shown in Table 7. Final purity was assayed by two HPLC techniques (Phenyl 5PW and BioSil SEC125) on fraction S6 (FIG. 20). The results are shown in FIGS. 21 and 22 for the chromoprotein preparation containing 10% apoprotein. Purity at each stage is also demonstrated by SDS-PAGE with Coomassie staining. (FIGS. 23 and 24). A single band is observed for the final pool (FIG. 24, lane 3).

### Example 10

### Example 10-Separation and Structural Elucidation of Chromophore Species.

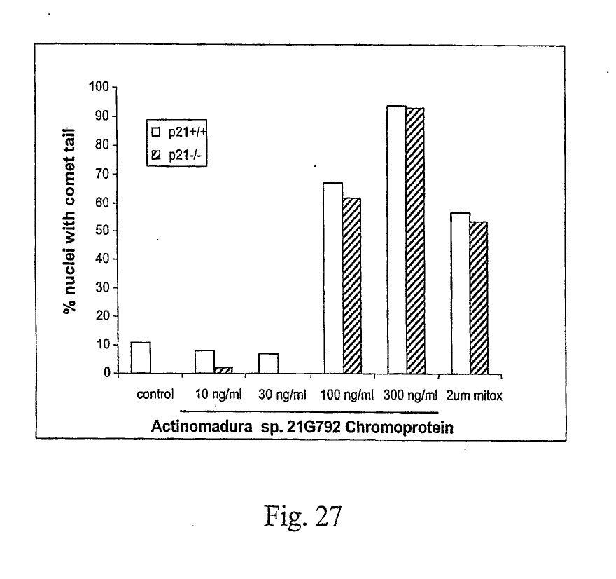

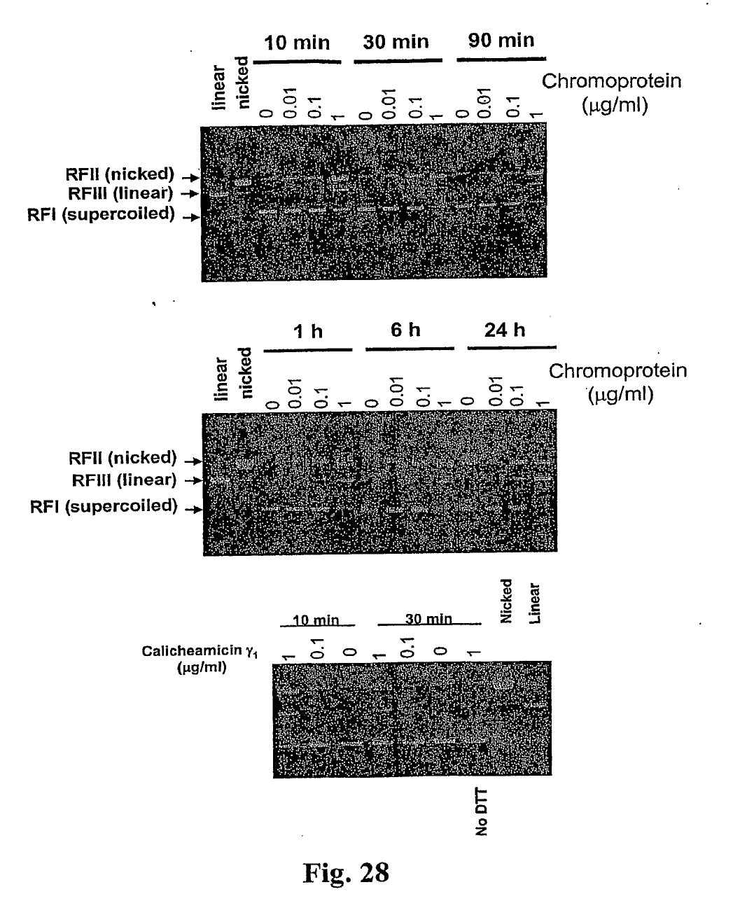

A fermentation culture of Actinomadura sp. 21G792 containing halide was prepared. Chromoprotein was obtained from clarified fermentation broth by sequential application and collection of active fractions from DEAE Sepharose FF, Phenyl Sepharose FF, and Superdex 75 chromatography columns. Separation of chromoproteins into active fraction peaks A and B and an inactive apoprotein peak was effected during the phenyl sepharose step using a binding buffer composed of 1.5 M ammonium sulfate and a programmed 20 CV gradient terminating in 0.1 M NaCl (FIG. 25). The three peaks were identified in the chromatograph (FIG. 25) and fractions corresponding to each of the peaks were pooled. The enediyne chromophore present in each fraction was analyzed by reversed phase chromatography using a Jupiter C4 300A 4.6×250 mm column (Phenomenex). The method relied on an in-line extraction of the enediyne from the apoprotein carrier component of the chromoprotein solution using an acetonitrile organic modifier (FIG. 26). Peak A yielded two chromophore species (chromophore-c and chromophore-d), whereas Peak B yielded a single chromophore species (chromophore-b).

All three chromophore species were isolated and their HRMS and NMR data were collected. Proposed structures were further confirmed by high resolution MS/MS fragmentation data. (FIG. 6). Chromophore-c and -d are potentially degradation products of chromophore-b that result from cyclo-aromatization to a diradical followed by protonation and dehydration (loss of water). To confirm that chromophore-c and -d were not artifacts produced during isolation, LC-NMR of the crude active fraction mixture was also conducted. The NMR data from LC-NMR accorded with the proposed structures.

### Example 11

### Actinomadura Sp. 21G792 Gene Cluster

### Example 11-DNA Isolation and Sequencing of the Actinomadura sp. 21G792 Apoprotein.

Genomic DNA was isolated from Actinomadura sp. 21G792 based on a modification to the procedure described in Hopwood et al. (1985), Genetic manipulations of Streptomyces. A Laboratory Manual. Norwich: John Innes Foundation. Approximately 1 ml of a frozen mycelia glycerol stock was inoculated into a 25 mm×150 mm seed tube containing 10 ml of MYM media (4 g/l maltose, 4 g/l yeast extract, 10 g/l malt extract, pH 7.0) and 2-6 mm glass beads. The culture was grown at 28° C. and 200 rpm for 5 days. The cells were then pelleted by centrifugation at 3000×g for 10 min. The supernatant was discarded and the pellet was suspended in 300 μl of T₅₀-E₂₀ (Tris 50 mM-EDTA-20 mM) containing 5 mg/ml lysozyme and 0.1 mg/ml RNase and incubated at 37° C. for 1 hr with gentle mixing every 15 min. 50 μl of 10% SDS was then added and the sample was thoroughly mixed. Next, 85 μl of 5 mM NaCl was added and the sample was again thoroughly mixed. The sample was then extracted with 400 μl phenol/chloroform/isoamyl alcohol (50/49/1). After vortexing the sample thoroughly, it was centrifuged at 10,000×g for 20 min at room temperature. Following centrifugation, the aqueous phase was removed and placed in a new microcentrifuge tube. An equal volume of room temperature isopropanol was added to the sample and thoroughly mixed by inversion. The sample was let stand at room temperature for 5 min. The sample was then centrifuged at 12,000×g for 30 min at 4° C. The isopropanol was carefully poured out of the tube and the DNA pellet rinsed with 1 ml of cold 70% ethanol. After being let stand in ice for 5 min, the 70% ethanol was poured out of the tube and the DNA was air dried for 10 minutes. The DNA was dissolved in 0.3 ml of sterile water. DNA integrity and concentration were estimated by agarose gel electrophoresis.

Escherichia coli; Plasmid and Small Scale Cosmid DNA preparations: Plasmid DNA and small-scale cosmid DNA preparations were performed using the Qiaprep Spin MiniPrep Kit (Qiagen Inc, Valencia, Calif., USA) according to the manufacturer's specifications. Cosmid: Cosmid DNA was isolated using the Qiagen Large Construct Kit (Qiagen Inc, Valencia, Calif., USA) according to the manufacturer's specifications.

An Actinomadura sp. 21G792 genomic library was constructed using the pWEB Cosmid Cloning Kit (Epicentre Technologies, Madison, Wis., USA) according to the manufacturer's specifications. The general library construction protocol was as follows. 10 μg of genomic DNA was randomly sheared into 30-45 kb fragments by passing the genomic DNA through a Hamilton HPLC/GC syringe. Following shearing, the fragmented DNA was end-repaired to produce blunt-ended fragments using the end-repair enzyme mix contained in the kit. The sheared and end-repaired DNA was then separated on a 1% low melting point agarose gel using linear T7 DNA (˜40 Kb) to serve as a molecular weight marker. Genomic DNA approximately equal in size to the T7 DNA was cut from the gel and the DNA was eluted from the agarose. The purified DNA was then ligated into the pWEB vector. Following ligation, the ligated insert DNA was packaged into lambda phage particles using the MaxPlax Lambda Packaging Extracts provided with the pWEB cosmid cloning kit. The phage extract was then titered to determine the colony-forming units per milliliter. Upon determining the titer of the phage extract, an appropriate amount of extract was used to infect E. coli EPI100 host cells and the infected cells were plated on Difco Luria agar plates containing 50 μg/ml of kanamycin to give a cell density of approximately 200 colonies per plate.

Library screening strategy and methodology; dNDP-glucose-4,6-dehydratase probe generation. Generally, the genes required to produce a particular antibiotic are clustered in the producing organism's genome. Further, there is precedence for clustering of an apoprotein gene with the genes encoding proteins involved in the biosynthetic pathway of the corresponding chromophore (Liu et al., 2002, Science 297:1170-3). The chromophore produced by Actinomadura sp. 21G792 contains the amino sugar 4-amino-4-deoxy-3-C-methyl-β-ribopyranose, which is attached to the enediyne core. Because a dNDP-D-glucose-4,6-dehydratase (DH) was expected to catalyze a step in the biosynthesis of this sugar, a DH probe was employed to isolate biosynthetic cluster.

To generate a DH probe, the polymerase chain reaction (PCR) was used to amplify a DH gene fragment from the genomic DNA of Actinomadura sp. 21G792. Primers for the expected ˜500 bp DH gene fragment (dehydra1: 5′-CSGGSGSSGCSGGSTTCATSGG (SEQ ID NO:152) and dehydra2: 5′-GGGWRCTGGYRSGGSCCGTAGTTG (SEQ ID NO:153)) were identical to those described by Decker et al., 1996, FEMS Microbiol. Lett. 141, 195-201. PCR was conducted using JumpStart REDTaq Ready Mix PCR Reaction Mix (Sigma-Aldrich Corp, St. Louis, Mo.) according to the manufacturer's specifications. The primers were used at a final concentration of 0.5 μM. PCR was performed on a Biometra T gradient thermocycler. The starting denaturing temperature was 96° C. for 4 min. The following 30 cycles were as follows: denaturing temperature 96° C. (45 sec), annealing temperature 66° C. (45 sec), extension temperature 72° C. (3 min). At the end, the final extension temperature was 72° C. for 10 min.

The ˜500 bp amplicon was cloned into pCR2.1 using the TOPO TA Cloning Kit (Invitrogen Corp, Carlsbad, Calif.) following the manufacturer's recommendations. A portion (2.5 μl) of the cloning reaction was used to transform E. coli TOP 10 cells (Invitrogen Corp, Carlsbad, Calif.) which were subsequently plated on Difco Luria Agar containing 50 μg/ml kanamycin, 40 μg/ml X-gal and 0.2 mM IPTG to facilitate blue/white screening of recombinant clones. Twenty white colonies were picked and their plasmid DNA was isolated. Sequencing of these clones revealed that two different DH gene fragments had been cloned. Comparison of the deduced amino acid sequences revealed that one of the DH fragments (contained in plasmid p34598) was most similar to a DH involved in calicheamicin biosynthesis. As the calicheamicin structure contains 2 amino sugars, it was predicted that the DH fragment contained in p34598 might also be involved in amino sugar production, and thus was chosen as the probe for the chromoprotein gene cluster.

Colony hybridization: The Actinomadura sp. 21G792 genomic library was screened by colony hybridization using the p34598 DH fragment. Recombinant colony DNA was transferred to Nytran SuPerCharge nylon membrane discs (Schleicher & Schuell BioScience, Inc., Keene, N.H.) as described by Sambrook and Russell (2001), Molecular Cloning, A Laboratory Manual, Cold Spring Harbor Laboratory Press (3^{rd} ed.). The DH probe was prepared using PCR and primers dehydra1 and dehydra2 to amplify the insert of p34598. The amplified PCR product was separated by agarose gel electrophoresis and the 530 bp fragment was isolated from the agarose. This fragment was then labeled with [α-³²P]dCTP (3000 Ci/mmol Amersham Bioscience, Piscataway, N.J.) using the Megaprime DNA Labeling kit according to the manufacturer's specifications (Amersham Bioscience, Piscataway, N.J.). The nylon membrane on which the DNA samples were immobilized was washed in 6×SSC, then placed in a hybridization bottle with prewarmed (65° C.) prehybridization solution (6×SSC/5×Denhardt's reagent/0.5% (w/v) SDS and 100 μg/ml of denatured, sheared herring sperm DNA) and “pre-hybridized” for 2 h. The denatured probe was then added, and hybridization proceeded overnight at 65° C. The following day the membrane was washed once with prewarmed (65° C.) 2×SSC/0.1% SDS (Wash Solution 1) for 1 h and once with prewarmed (65° C.) 1×SSC/0.1% SDS (Wash Solution 2) for 1 h. The nylon membrane was then wrapped in Saran wrap and exposed to Kodak X-omat AR film for 4 h. The exposed films were developed using a Kodak X-omat 2000A processor. Twenty-two colonies appeared to hybridize to the probe. These colonies were picked and grown in Difco Luria Broth containing 50 μg/ml kanamycin. The cosmid DNA was purified from the cultures and cut with Not I. The restriction digests were separated by agarose gel electrophoresis and the DNA was transferred to a Nytran SuPerCharge nylon membrane as described by Sambrook and Russell (2001). This membrane was probed using the same conditions used for the colony hybridization, again using the p34598 insert as a probe. Nine cosmids positively hybridized to the probe. The cosmids and approximate sizes of the fragments that hybridized to the probe were: 21gB: 15-20 kb, 21gC: 15-20 kb, 21gD: 8-12 kb, 21gF: 15-20 kb, 21gG: 3-4 kb, 21gI: 1.2-2.5 kb, 21gK: 15-20 kb, 21gL: 2.5-3 kb, 21gV: 2-2.5 kb.

Apoprotein—specific oligonucleotide probe hybridization: Edman protein sequencing was used to determine the first 38 amino acid residues of the apoprotein, N-terminus DTVTVNYDDVGYPSDIAVTIDAPATAGVGDTATFEVSV (SEQ ID NO:154). To definitively identify which cosmids might contain the apoprotein gene sequence, a hybridization experiment was conducted using, as a probe, a degenerate oligonucleotide that was based on residues 4-12 of the 38 amino acid (aa) sequence of the apoprotein N-terminus. Specifically, the sequence of the oligonucleotide was 5′-ACSGTSAACTACGACGACGTSGGNTAC (SEQ ID NO:155).

The cosmids that hybridized to the DH probe were digested with Not I and transferred to a Nytran SuPerCharge nylon membrane. The oligonucleotide was end labeled with [γ-³²P]dATP (6000 Ci/mmol; Amersham Bioscience, Piscataway, N.J.) using the KinaseMax 5′ End-Labeling Kit according to the manufacturer's recommendations (Ambion Inc., Austin, Tex.). Unincorporated radioactive nucleotides were removed using the NucAway Spin Column Kit according to the manufacturer's directions (Ambion Inc., Austin, Tex.). The DNA-carrying nylon membrane was “pre-hybridized” for 3 h at 50° C. in a solution containing 6×SSC, 5×Denhardt's reagent, 0.05% sodium pyrophosphate, 0.5% SDS and 100 μg/ml sheared and denatured salmon sperm DNA. Following this step, the pre-hybridization solution was replaced with 7 ml pre-warmed (50° C.) hybridization solution containing 6×SSC, 0.5% sodium phosphate, 1×Denhardt's reagent and 100 μg/ml yeast tRNA. The labeled probe was added to this solution and the hybridization was incubated at 50° C. for 22 h. Next, the hybridization solution was discarded and the membrane was rinsed briefly with 20 ml of room temperature TMACL wash buffer (3 M TMACL, 50 mM Tris, 0.2% SDS). It was then washed with an additional 50 ml of pre-warmed (67° C.) TMACL wash buffer for 55 min at 67° C. For the final wash, the membrane was washed with 50 ml of pre-warmed (50° C.) Wash Solution 1 for 10 min at 50° C. The membrane was then wrapped in Saran wrap and exposed to Kodak X-omat AR film for 24 h.

Cosmids 21gD, 21gG and 21gK hybridized to the probe. An ˜4.5 kb signal was observed in the lanes containing 21gD and 21gK DNA, while an ˜5.2 kb signal was observed in the lane containing 21gG DNA. To confirm this hybridization result, PCR was conducted using 21gD cosmid DNA as the template and degenerate PCR primers designed to amplify a 98 bp fragment from the apoprotein. The PCR primers CP-FWD3 (5′-ACSGTSAAYTAYGAYGAYGT; SEQ ID NO:156) and CP-REV4 (5′-ACYTCRAASGTSGCSGTRTC; SEQ ID NO:157) were designed using the reverse translated DNA sequence deduced from the 36 aa sequence of the apoprotein. PCR was performed using JumpStart REDTaq Ready Mix PCR Reaction Mix (Sigma-Aldrich Corp, St. Louis, Mo.) according to the manufacturer's specifications. The primers were used at a final concentration of 2.0 μM. The PCR was performed on a Biometra Tgradient thermocycler. The starting denaturing temperature was 96° C. for 4 min. The following 5 cycles were as follows: denaturing temperature 96° C. (45 sec), annealing temperature 40° C. (45 sec), extension temperature 72° C. (2 min). The next 30 cycles were as follows: denaturing temperature 96° C. (30 sec), annealing temperature 55.7-72.0° C. (45 sec; 8 temperatures tested within range), extension temperature 72° C. (2 min). At the end, the final extension temperature was 72° C. for 10 min. Several bands were generated by these conditions; however, using annealing temperatures 55.7° C., 58.6° C. and 61.4° C., an intense band of approximately 100 bp was generated. The ˜100 bp amplicon was cloned into pCR2.1 using the TOPO TA Cloning Kit (Invitrogen Corp, Carlsbad, Calif.) following the manufacturer's recommendations. A portion (2.5 μL) of the cloning reaction was used to transform E. coli TOP10 cells (Invitrogen Corp, Carlsbad, Calif.) which were subsequently plated on Difco Luria Agar containing 50 μg/ml kanamycin, 40 μg/ml X-gal and 0.2 mM IPTG to facilitate blue/white screening of recombinant clones. Ten white colonies were picked and their plasmid DNA isolated. Sequencing of these clones revealed that 4 clones (p35546, p35547, p35550, p35554) contained DNA whose deduced amino acid sequence matched that of the 36 aa apoprotein fragment exactly, thus confirming that the gene encoding the apoprotein was contained in cosmid 21gD.

Elucidation of complete apoprotein DNA sequence in cosmid 21gD. To determine the full sequence of the gene encoding the apoprotein, sequencing primers were designed from the DNA sequence of the 98 bp PCR product amplified above. The following primers were used for the initial round of sequencing using cosmid 21gD as a template: ApoSeqCode1: 5′-GGCTACCCGTCGGACATCG (SEQ ID NO:158); ApoSeqCode2: 5′-GGACATCGCCGTGACCATCG (SEQ ID NO:159); ApoSeqComp1: 5′-CCGGCGCGTCGATGGTCAC (SEQ ID NO:160); ApoSeqComp2: 5′-CTCGAAGGTGGCGGTGTC (SEQ ID NO:161).

The first round of sequencing generated ˜1440 bp of sequence. Using the CodonPreference program, a small 498 bp open reading frame (ORF) was identified. Comparison of the deduced amino acid sequence of this orf to the partial amino acid sequence of the Actinomadura sp. 21G792 apoprotein (determined by Edman protein sequencing) confirmed that the ORF did encode the apoprotein, as the two amino acid sequences were identical. Additionally, the molecular weight of the deduced amino acid sequence, 12926 Da, was in good agreement with the molecular weight of the apoprotein as determined by high resolution MALDI MS, 12924.09. Also, the DNA sequence of the apoprotein was confirmed further by extensive sequencing of both DNA strands using primers flanking the orf encoding the apoprotein (designated aseA).

The deduced amino acid sequence of the pre-apoprotein, which contains the leader peptide and the apoprotein, is provided in SEQ ID NO:64. The nucleotide sequence encoding the pre-apoprotein is provided in SEQ ID NO:63. The deduced amino acid sequence of the apoprotein is provided in SEQ ID NO:150. The nucleotide sequence encoding the apoprotein is provided in SEQ ID NO:149. Finally, a figure describing the DNA sequence of the pre-apoprotein, the corresponding amino acid sequence, the putative upstream ribosome binding site, and the splitting site between the leader peptide and apoprotein is provided in FIG. 7.

### Example 12

### Example 12-DNA Isolation and Sequencing of the Remainder of the Actinomadura sp. 21G792 Chromoprotein Biosynthetic Cluster

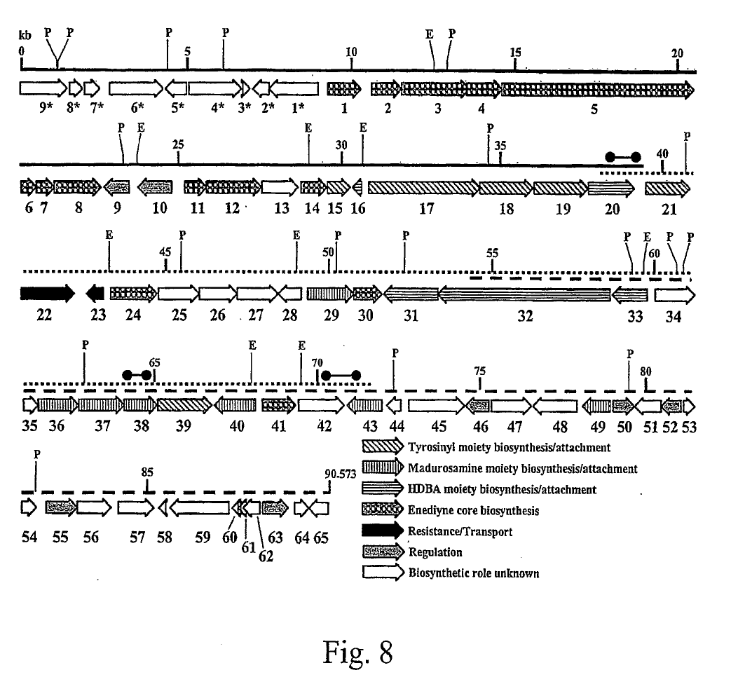

Identification of distal sequences of the Actinomadura sp. 21G792 apoprotein gene cluster. Sequences adjacent to the portion of the Actinomadura sp. 21G792 apoprotein gene cluster present in cosmid 21gD were identified as described below. Along with cosmid 21gD, these sequences are thought to constitute substantially the entire biosynthetic cluster of the Actinomadura sp. 21G792 chromoprotein—i.e. the genes responsible for assembling the chromoprotein Locations of the open reading frames are identified in Table 1. Functions of the encoded proteins were deduced by comparison with GenBank sequence deposits (Table 3). The arrangement of the open reading frames is depicted in FIG. 8.

First, a probe was generated from cosmid 21gD by amplifying a 904 bp fragment from the end of the cosmid containing the partial type II peptide synthetase condensation domain (orf20; FIG. 7) using primers 21gDpr1FWD (5′-GCTCGTCGGGTTCTTCTAC; SEQ ID NO:162) and 21gDpr1REV (5′-GACTTCGCGATAGCTCTC; SEQ ID NO:163). PCR amplification was conducted using KOD polymerase (Novagen) with 5% DMSO according to the manufacturers recommendations. Primers were used at a concentration of 0.5 mM. Cosmid 21gD was used as template DNA. The cycling conditions were as follows: 1 cycle of 96° C. for 2 min, followed by 30 cycles of 96° C. for 1 min, 61.2° C. for 1 min, and 72° C. for 2 min, followed by 1 cycle of 72° C. for 10 min. The PCR reaction was examined by agarose gel electrophoresis and the 904 bp band was eluted from the agarose as previously described. The 904 bp amplicon was used to probe the Actinomadura sp. 21G792 genomic cosmid library as previously described for the 4,6-dehydratase probe. 38 colonies that hybridized to the probe were cultured (5 ml Difco Luria Broth containing 50 μg/ml kanamycin) and cosmid DNA was purified. The purified cosmids were end sequenced using sequencing primer sites contained in the pWEB vector. Analysis of the DNA sequences indicated that one cosmid (41417) overlapped with cosmid 21gD by 1184 bp. Cosmid 41417 was subsequently sequenced in its entirety, open reading frames were identified, and functions of the encoded proteins were deduced.

The portion of the biosynthetic cluster distal to the other end of cosmid 21gD was identified by screening the cosmids previously identified as having hybridized to the putative dNDP-D-glucose-4,6-dehydratase fragment cloned in p34598 (used to identify cosmid 21gD). These cosmids were screened using PCR primers designed to amplify a 1043 bp product from the 5′ end of cosmid 21gD (product corresponds to nucleotides 70,572 to 71,614 of the complete biosynthetic cluster). The primers 21gDendFWD (5′-GCGACGAAGGACCCGAAGG; SEQ ID NO:164) and 21gDendREV (5′-CACGCTGGCCCGCCCCTTC; SEQ ID NO:165) were used to screen each of the cosmids using 10-100 ng of each cosmid as template in a standard 25 μl PCR reaction (KOD Hot Start polymerase; Novagen, San Diego, Calif., USA) along with 0.5 μM of each primer. The only cosmids that supported amplification of the expected 1043 bp DNA fragment were cosmids 21gB and 21gC. End sequencing of these cosmids revealed that cosmid 21gB overlapped cosmid 21gD by 17,411 nucleotides, while cosmid 21gC overlapped cosmid 21gD by 22,796 nucleotides. Since cosmid 21gB overlapped less with the known cluster sequence, and thereby represented a greater potential for yielding a longer sequence extension than cosmid 21gC, it was chosen for sequencing. Sequencing revealed that cosmid 21gB contained a 33,133 bp insert which represented a 18,442 bp sequence extension, bringing the total number of base pairs sequenced to 90,573 (FIG. 8). As before, the cosmid was sequenced, open reading frames were identified, and functions of the encoded proteins were deduced.

### Example 13

### Biological Properties of the 21G792 Chromoprotein

### Example 13-In Vitro Anti-Tumor Activity.

The p53/p21 checkpoint monitors the integrity of the genome and blocks cell cycle progression in the event of DNA damage. Disruption of the checkpoint by deletion of the p21 gene results in failure to arrest in response to DNA damage ultimately leading to cell death through apoptosis. Since loss of this checkpoint is a hallmark of cancer cells, an isogenic pair of cell lines, wherein one pair of the cell line (p21+/+) has an intact p21 gene and one member (p21−/−) has a deletion in the p21 gene, can be used to screen for potential anti-tumor compounds by identifying molecules that preferentially induce apoptosis in p21-deficient cells.

The Actinomadura sp. 21G792 chromoprotein was added to an isogenic pair of cell lines (p21+/+ and p21−/−). As shown in Table 8, the chromoprotein was highly selective for p 21−/− cells, as the IC₅₀ was 13-fold higher for p21+/+ cells. Also, as shown in Table 9, the chromoprotein showed excellent potency in a human tumor cell line panel, as the IC₅₀ ranged from 1 to 47 ng/ml. The apoprotein alone, however, was inactive.

TABLE 8
SENSITIVITY OF P21−/− CELLS TO ACTINOMADURA
SP. 21G792 CHROMOPROTEIN

| | |
|---|---|
| Isogenic cell lines | Selectivity |

| | p21+/+ | p21−/− | Ratio |
|---|---|---|---|
| IC₅₀ (μg/ml) | 90 + 32 | 7 + 2 | 13 |

Mean + SD, n = 3

TABLE 9
POTENCY OF ACTINOMADURA SP. 21G792 CHROMOPROTEIN
AGAINST HUMAN TUMOR CELL LINES

| Tumor Cell Line | Tissue | IC₅₀ (μg/ml) |
|---|---|---|
| DLD1 | Colon | 8 |
| HCT116 | Colon | 1 |
| HT29 | Colon | 8 |
| LoVo | Colon | 2 |
| SW620 | Colon | 2 |
| BT474 | Breast | 47 |
| MCF-7 | Breast | 2 |
| MDA-MB-361 | Breast | 5 |
| HN5 | Head & Neck | 4 |
| LOX | Melanoma | 1 |
| PC3 | Prostate | 22 |

### Example 14

### Example 14-DNA Damage Induced by the Chromoprotein.

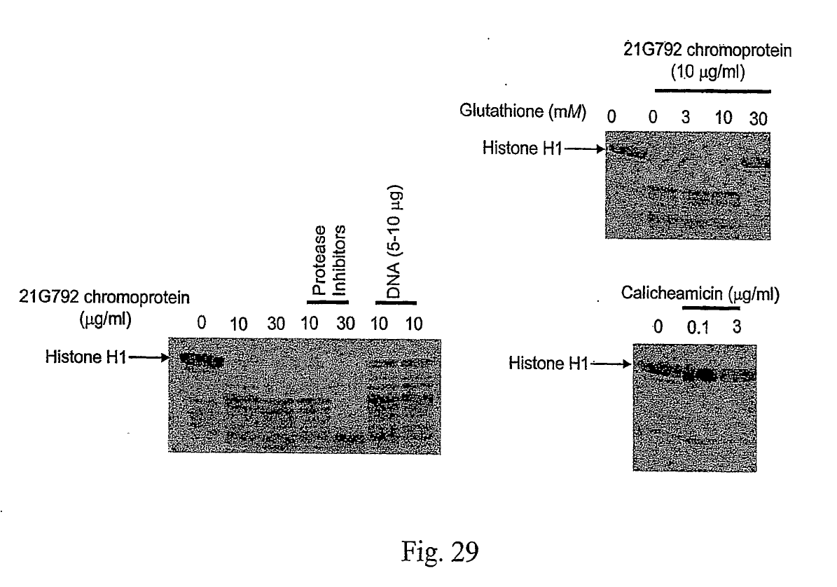

A COMET assay obtained from Trevigen, Inc. was used to detect DNA damage. HCT116 p21+/+ and −/− cells were subjected to various amounts of the 21G792 chromoprotein and mitoxantrone. As shown in FIG. 27, the chromoprotein induced dose-dependent DNA strand breaks occur in both p21-proficient and p21-deficient cells at >100 ng/ml concentrations.

### Example 15

### Example 15-DNA Cleavage Induced by the Chromoprotein.

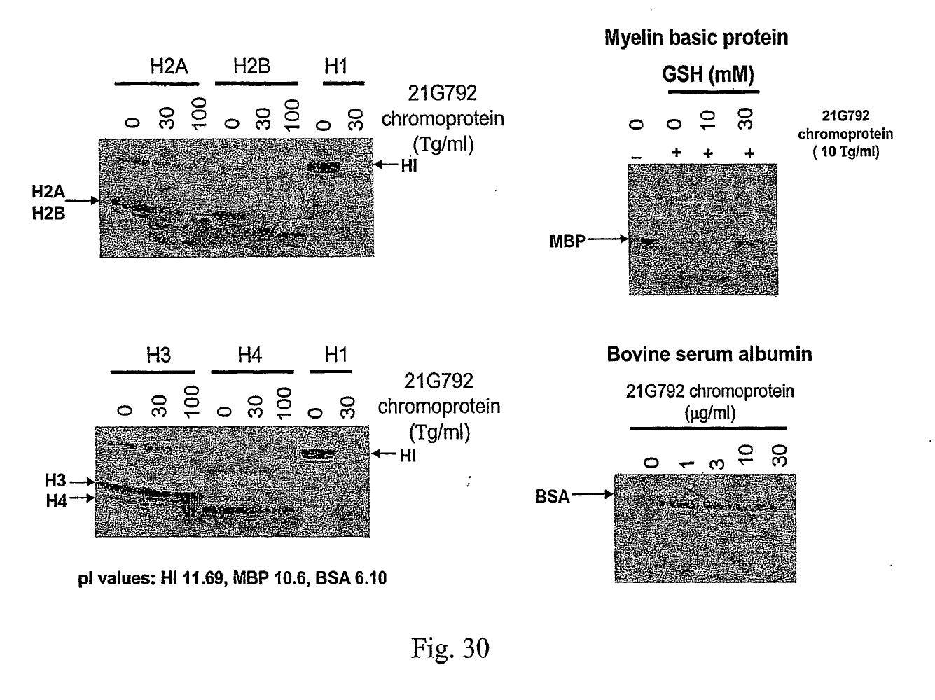

Supercoiled φX174 DNA was incubated with various concentrations of the 21G792 chromoprotein and analyzed by gel electrophoresis. It was observed that the chromoprotein induced single strand breaks and double strand breaks, the reaction continued to progress over 24 hours, and DNA cleavage did not require a reducing agent (dithiothreitol, DTT), unlike calicheamicin. The gel electrophoresis is shown in FIG. 28. Nicked refers to single strand breaks in the DNA and linear refers to double strand breaks.

### Example 16

### Example 16-Digestion of Histone H1 by the Chromoprotein.

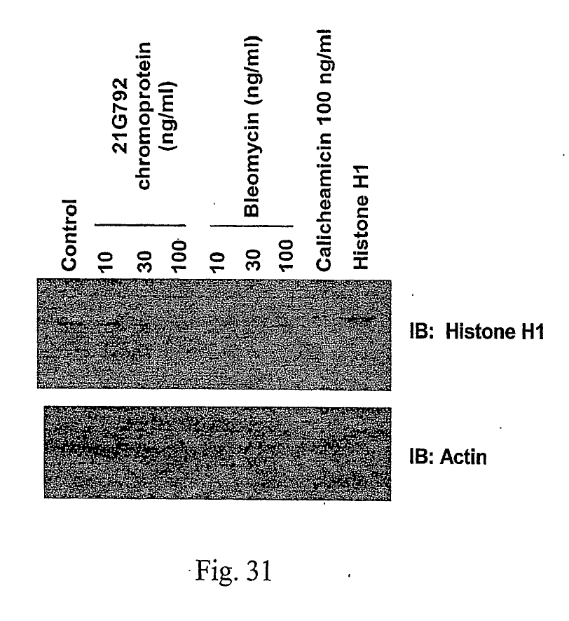

Chromoprotein enediynes have previously been shown to cleave histones (Zein et al., 1993, Proc. Natl. Acad. Sci. USA 90, 8009-12; Zein et al, 1995, Chem & Biol 2, 451-5; Zein et al., 1995, Biochem 34, 11591-7), and although this activity is controvorsial (Heyd et al., 2000, J. Bacteriol. 182, 1812-8), it was presumed to be due to a proteolytic activity of the apoprotein. Histone H1 was incubated with various concentrations of the chromoprotein in 50 mM Tris-Cl, pH 7.5 overnight at 37° C. (FIG. 29) Digestions of histone were assessed by SDS-polyacrylamide gel electrophoresis (SDS-PAGE), followed by staining of the gel with GelCode Blue (Pierce Biotechnology, Inc, Rockford, Ill.). Digestion of histone HI was inhibited by addition of DNA, indicating that the same mechanisms required for DNA cleavage (e.g., a free-radical based mechanism) are also involved in digesting proteins. Consistent with this, digestion of histones was inhibited by the addition of free radical scavengers, 30 mM glutathione or N-acetyl cysteine (not shown), but not by protease inhibitors. Calicheamicin, a non-protein-containing enediyne, did not cleave histone H1, indicating the requirement of an intact chromophore-protein complex for this activity.

### Example 17

### Example 17-Specificity of Digestion by the Chromoprotein.

The order of preference of digestion of histones by the chromoprotein is H1>H2A>H2B>H3>H4 (FIG. 30). The chromoprotein also cleaves other basic proteins such as myelin basic protein, but not neutral/acidic proteins such as bovine serum albumin. This can explain the requirement of the apoprotein component of the chromophore for histone cleaving activity: the acidic apoprotein may deliver the chromophore to histones and other basic proteins by electrostatic interaction, allowing the chromophore to cleave the basic proteins by a free-radical based mechanism.

### Example 18

### Example 18-Digestion of Histone H1 in Hela Cells by the Chromoprotein.

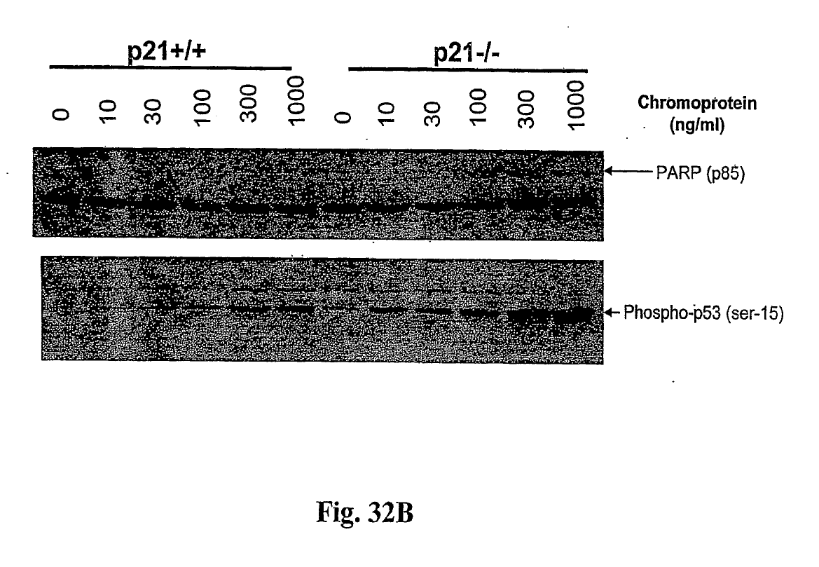

To study whether the digestion of histones by the chromoprotein occurs in intact cells, HeLa cells were incubated with compounds overnight at 37° C. Cell lysates were analysed by SDS-PAGE and protein immunoblotting using anti-histone H1 antibodies (Santa Cruz Biotechnologies). Incubation of cells with the chromoprotein resulted in reduced histone H1 in cells (FIG. 31). No effect was observed with bleomycin, another DNA damaging agent, or with calicheamicin. This demonstrates that the chromoprotein is capable of digesting histones within intact cells. This activity can contribute to antitumor effects by digesting histones in chromatin, making the DNA more accessible for cleavage. This appears to be a unique activity of the chromoprotein enediynes.

### Example 19

### Example 19-Chromoprotein Induction of the G1/S Checkpoint.

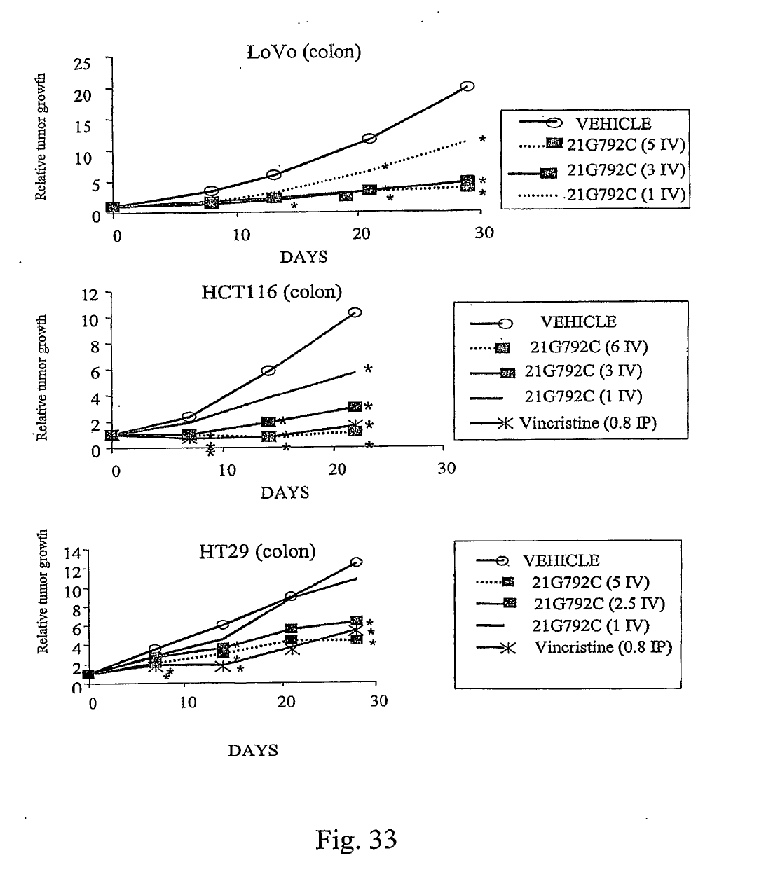

HCT116 (p21+/+ and p21−/−) cells were exposed to the chromoprotein at various concentrations. As shown in FIG. 32A, exposure to the chromoprotein resulted in the activation of the p53 checkpoint for all tested concentrations. Induction of the p21 protein was seen in the p21+/+ cells only. Activation of the DNA damage checkpoint by the Actinomadura sp. 21G792 chromoprotein was confirmed by demonstrating phosphorylation of the serine-15 amino acid residue in p53, which is known to be important for the transcriptional activation of the p53 protein (FIG. 32B). Furthermore, induction of apoptosis was preferentially observed in p21−/− cells compared with p21+/+ cells, when treated with the Actinomadura sp. 21G792 chromoprotein as shown by the cleavage of poly ADP ribose phosphorylase (PARP) (FIG. 23B). This is consistent with the lower IC50 value in the p21−/− cells.

### Example 20

### Example 20-Activity of Chromoprotein Species.

In two experiments, the activities of peak “A” (chromoproteins comprising chromophore-c and -d) peak “B” (chromoprotein comprising chromophore-b) and preparations of chromoproteins comprising all chromophore isoforms were determined in HCT116 colon carcinoma cells and in 80S14 cells (a p21−/− variant of HCT116). The potency of chromoprotein c (chromoprotein-d does not have significant activity) was consistently about one third the potency of chromoprotein b. For both chromoprotein-b and -c induction of apoptosis was preferentially observed in p21−/− cells compared with p21+/+ cells; see Table 10.

TABLE 10
ACTIVITIES OF CHROMOPROTEIN PREPARATIONS

| | Expt. 1 | | | Expt. 2 | | |
|---|---|---|---|---|---|---|
| | IC₅₀ (μg/ml) | | | IC₅₀(μg/ml) | | |
| Chromophore | HCT116 | 80S14 | | HCT116 | 80S14 | |
| preparation | p21+/+ | p21−/− | Ratio | p21+/+ | p21−/− | Ratio |
| unseparated | 0.059 | 0.014 | 4 | 0.044 | 0.005 | 9 |
| b | 0.172 | 0.048 | 4 | 0.210 | 0.033 | 6 |
| c | 0.064 | 0.016 | 4 | 0.066 | 0.011 | 6 |
| unseparated | | | | 1.020 | 0.024 | 43 |
| unseparated | | | | 0.097 | 0.008 | 12 |

### Example 21

### Example 21-In Vivo Anti-Tumor Activity

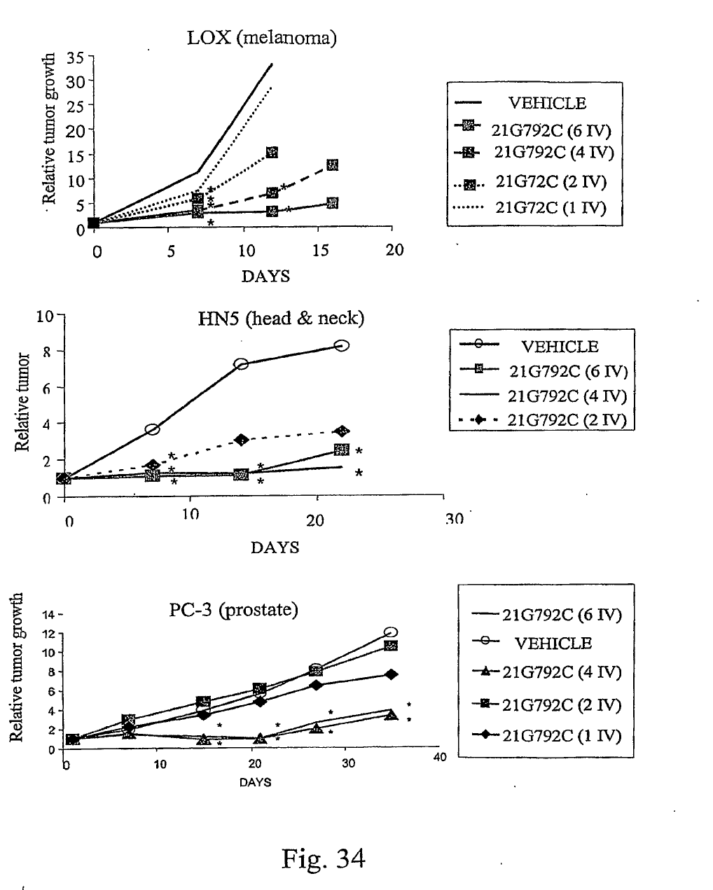

The human tumor cell lines or fragments LoVo (colon cancer); HCT116 (colon); HT29 (colon); LOX (melanoma); HN5 (head & neck); and PC-3 (prostate) were implanted under the skin of athymic (nude) mice and allowed to form a tumor mass. When the tumors reached a size of 90-200 mg, the saline control vehicle or various concentrations of the Actinomadura sp. 21G792 chromoprotein formulated in saline was administered intravenously to the mice. The mice received subsequent doses on days 5 and 9 and the relative tumor growth was observed. The results are shown in the graphs in FIG. 33 and FIG. 34. Inhibition of tumor growth of up to 80% for mice receiving the chromoprotein was observed.

### Example 22

### Example 22-Toxicity of the Chromoprotein

Toxicology studies suggest that, except for bone marrow suppression, the Actinomadura sp. 21G792 chromoprotein is well-tolerated in nude mice. Specifically, saline control vehicle or the chromoprotein in various doses was administered intravenously to six nude mice on days 1, 5, and 9. Microscopic studies of the mice showed that all mice receiving the chromoprotein exhibited bone marrow necrosis, with the mice receiving the most chromoprotein exhibiting the most severe lesions. A clinical pathology experiment revealed that mice receiving the most chromoprotein exhibited the lowest number of white blood cells and lymphocytes. No adverse effects, however were observed in the intestine, nerves, spinal cord, liver, or at the site of injection. The microscopic finding and clinical pathology summaries are provided in Tables 11 and 12.

TABLE 11
MICROSCOPIC FINDING SUMMARY

| | | Dose | Bone Marrow |
|---|---|---|---|
| Group | Treatment | (mg/kg) | Necrosis^{a} |
| 1 | Vehicle | 0 | 0/6 |
| 2 | 21G792 | 3 | 6/6 (1.7) |
| 3 | 21G792 | 6 | 6/6 (3) |

^{a}number with lesion/total number examined(x): average lesion severity where 0 = WNL, 1 = slight, 2 = mild, 3 = moderate, 4 = marked, 5 = severe

TABLE 12
CLINICAL PATHOLOGY

| | | Dose | WBC | Lymphocytes |
|---|---|---|---|---|
| Group | Treatment | (mg/kg) | (cells/μl) | (cells/μl) |
| 1 | Vehicle | 0 | 5100 | 3900 |
| 2 | 21G792 | 3 | 1430 | 290 |
| 3 | 21G792 | 6 | 1280 | 40 |

### Example 23

### Example 23-Transport of the Chromoprotein by P-GP (MDR-1)

Human PGP (MDR1) is an ATP-dependent efflux pump which is capable of transporting many drugs across cell membranes. High level expression of this protein has been linked to multiple drug resistance of tumors. As shown in Table 13, the Actinomadura sp. 21G792 chromoprotein is a poor MDR1 substrate, and cells expressing clinically relevant levels of MDR1 (KB-8-5 cells) remain sensitive to the complex. Notably, calicheamicin, which does not have a protein component, is a good substrate for MDR1. The protein component of the chromoprotein probably protects the chromophore from drug efflux mediated by MDR1, and may be responsible for the beneficial antitumor effects in colon cell lines which often express MDR1.

TABLE 13
IC₅₀ OF ACTINOMADURA SP. 21G792 CHROMOPHORE AND
CALICHEAMICIN AGAINST P-GP EXPRESSING CELLS

| | |
|---|---|
| Cell | IC₅₀ (ng/ml)^{a} |

| Line | P-GP Levels | 21G792 | Calicheamicin |
|---|---|---|---|
| KB | − | 10 | 3 |
| KB-8-5 | + | 6 | 21 |
| KB-V-1 | +++ | 142 | >1000 |

^{a}mean of two independent experiments

### Example 24

### Example 24-Uptake of FITC-Tagged Chromoprotein in HCT116 Cells

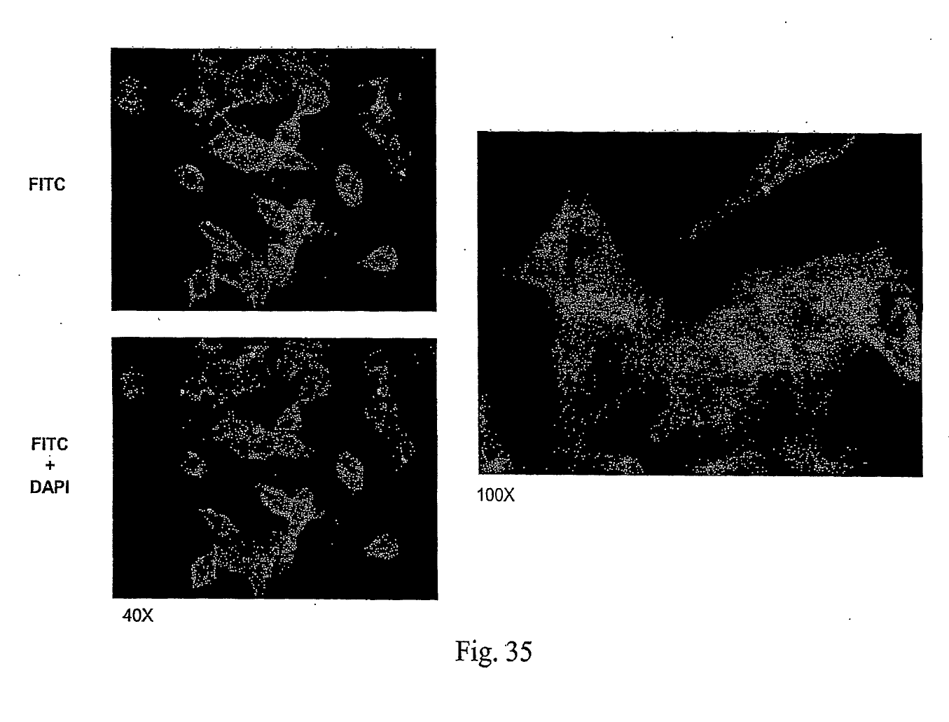

To determine the mechanism by which the chromoprotein enters cells and exerts its biological activity, the chromoprotein was labeled with a fluorescent tag (FITC) using EZ-Label fluorescent labeling kit (Pierce Biotechnology), according to the manufacturer's recommendation. No loss of biological activity was observed upon labeling. Uptake of labeled material by HCT116 colon carcinoma cells was studied by fluorescent microscopy. Optimum incubation time with cells was 3-6 hours. Most of the label appeared in the cytoplasm, although weak staining was also observed in the nucleus (FIG. 35). Even though nuclear accumulation is low, the amount is most likely sufficient for biological activity given the potency of the complex.

### Example 25

### Example 25-Uptake of FITC-Tagged Apoprotein and Chromoprotein in HCT116 Cells

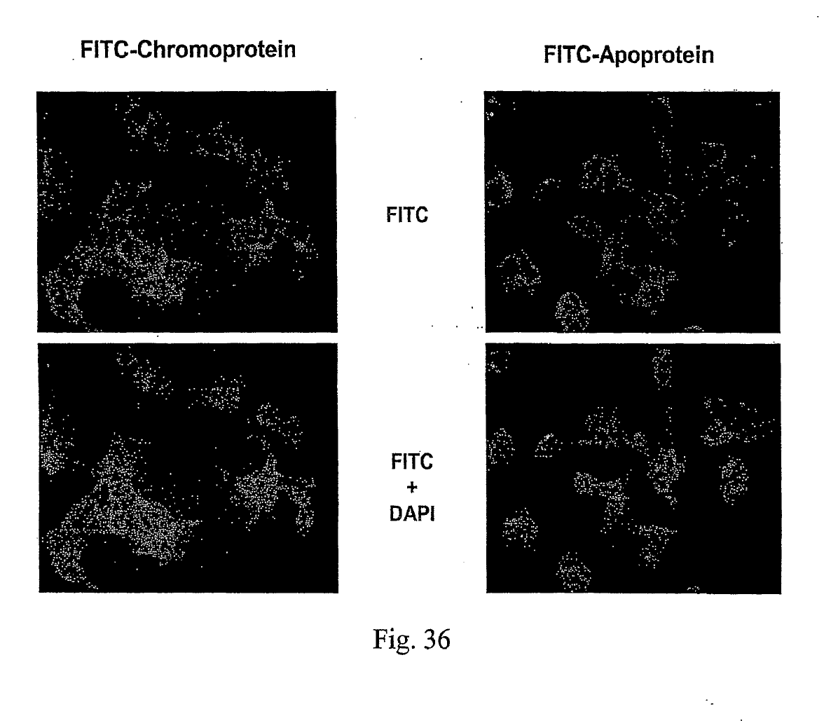

To determine whether an intact complex of chromophore and apoprotein is required for cellular and nuclear entry, the chromoprotein and apoprotein were labeled with FITC. Uptake of labeled material was studied by fluorescent microscopy. Uptake was similar for both apoprotein and chromoprotein. (FIG. 36), suggesting that cellular entry is not dependent on an intact chromophore-protein complex.

### Example 26

Example 26-Uptake of FITC-Tagged Chromoprotein: Competition with Unlabeled Complex

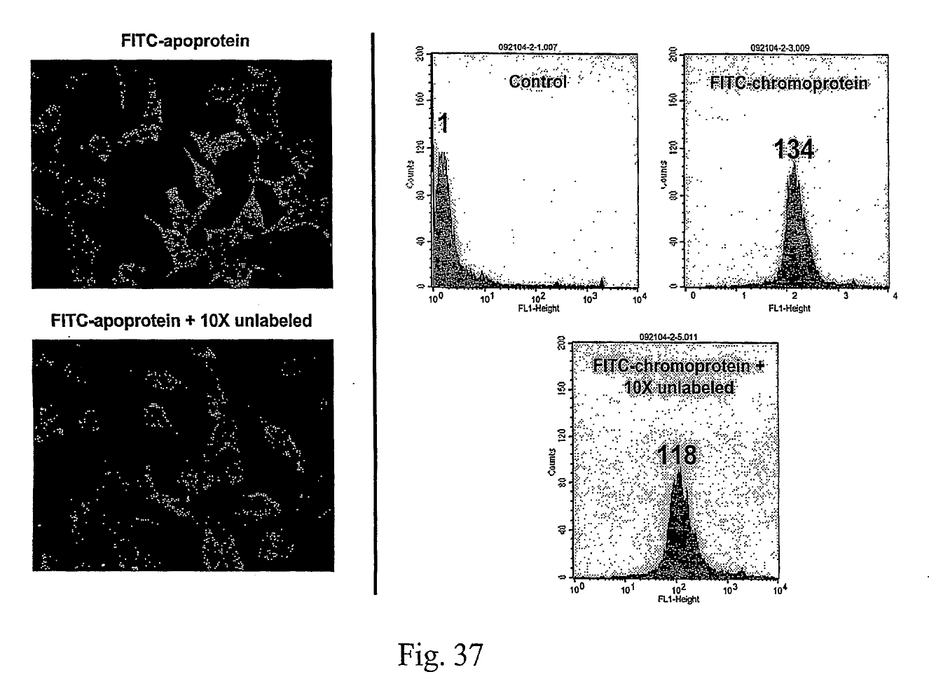

To determine whether the entry of chromoprotein into cells is mediated by a saturable (e.g. cell surface receptor-dependent) process, HCT116 cells were incubated with FITC-labeled chromoprotein (FIG. 37, right panel) or apoprotein (FIG. 37, left panel) in the absence or presence of 10-fold excess of unlabeled reagent (unlabelled chromoprotein or apoprotein, respectively). Cells were analysed by fluorescent microscopy (left) or flow cytometry (right). No competition of label was observed, suggesting that uptake of labeled material was not a receptor-mediated process. Furthermore, a single homogeneous peak observed in flow cytometry histograms indicated uniform uptake of labeled reagent by all cells. Numbers in the histograms are mean channel numbers (FITC fluorescence).

### Example 27

### Example 27-Effect of Energy Depletion and Microtubule Disruption on Uptake of FITC-Tagged Apoprotein by HCT116 Cells.

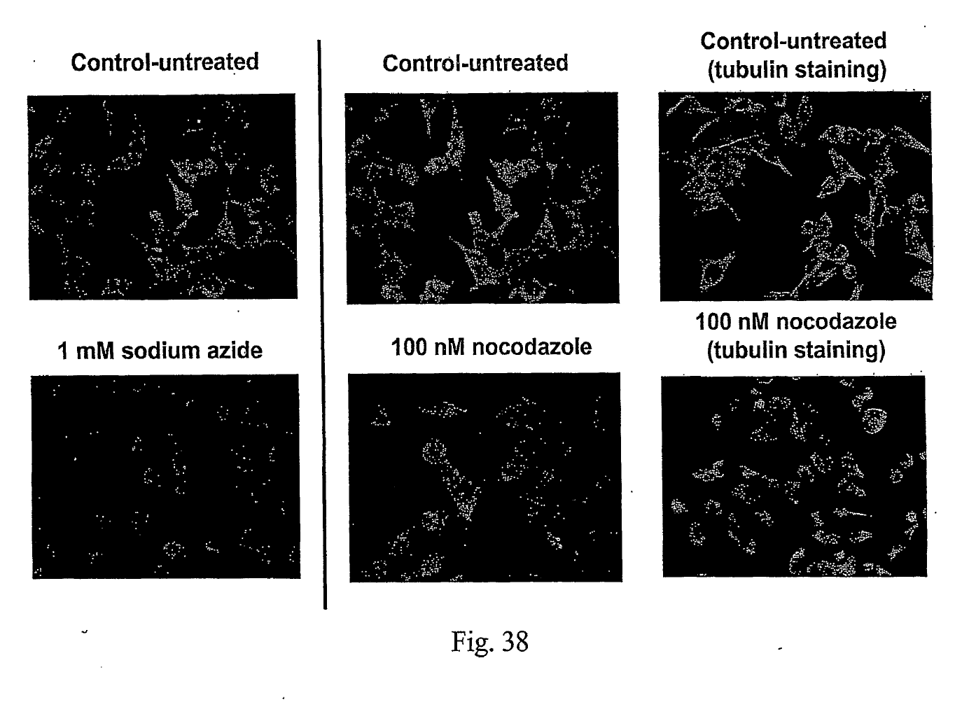

The above experiments suggest that entry of chromoprotein into cells is not a receptor-mediated process. Other means by which a protein complex can enter cells is pinocytosis, where caveolae in the surface of the cell pinch off to form pinosomes that are free within the cytoplasm of the cell. Since pinocytosis is an energy-dependent process that requires a functional tubulin cytoskeletal network, we examined the effect of sodium azide, an energy uncoupling agent and nocodazole, an agent which disrupts the tubulin cytoskeleton on cellular uptake. HCT116 cells were treated with FITC-labeled apoprotein in the absence or presence of sodium azideor nocodazole. Both treatments inhibited uptake of label (FIG. 38). The concentration of nocodazole (100 nM) was shown to be sufficient to disrupt microtubules (right panels). These data suggest that uptake of apoprotein is an energy-dependent process utilizing the microtubule network. Since the data appears to rule out a receptor-mediated process, pinocytosis is most likely involved.

1. A fermentation culture comprising an actinomycete that produces a chromoprotein, a halide selected from bromide and iodide, and a surfactant selected from Pluronic L-61, Pluracol P2000 (polypropylene glycols) and Antarox 17-R2.

2. The fermentation culture of claim 1, wherein the actinomycete is Actinomadura sp. 21G792.

3. The fermentation culture of claim 1, wherein the surfactant is Pluronic L-61.

4. The fermentation culture of claim 1, wherein the actinomycete is Actinomadura sp. 21G792 and the surfactant is Pluronic L-61.

5. The fermentation culture of claim 4, wherein Pluronic L-61 is present in an amount from about 0.1 to 10 g/L.

6. The fermentation culture of claim 5, wherein Pluronic L-61 is present at about 5 g/L.

7. The fermentation culture of claim 1, wherein the halide is present at a concentration of about 1-15 mM.

8. The fermentation culture of claim 1, wherein the halide is present at a concentration of about 3-5 mM.

9. A method of producing a chromoprotein comprising fermenting Actinomadura sp. 21G792 in a medium comprising a halide selected from bromide and iodide, and a surfactant selected from Pluronic L-61, Pluracol P2000 (polypropylene glycols) and Antarox 17-R2.

10. A method of purifying a chromoprotein of Actinomadura sp. 21G792 comprising obtaining a fluid comprising the Actinomadura sp. 21G792 chromoprotein, and contacting the fluid with one or more of
a) an anion exchange chromatography matrix,
b) a hydrophobic interaction chromatography matrix, or
c) a size exclusion chromatography matrix,
and recovering the purified chromoprotein.

11. A method of purifying a chromoprotein of Actinomadura sp. 21G792 comprising: (a) obtaining a fluid comprising the Actinomadura sp. 21G792 chromoprotein, (b) contacting the fluid with an anion exchange chromatography matrix, (c) contacting the fluid with a hydrophobic interaction chromatography matrix, (d) contacting the fluid with a size exclusion chromatography matrix, and (e) recovering the purified chromoprotein.

12. The method of claim 11, wherein said chromoprotein is recovered at a purity of about 80% or greater based on total protein.

13. The method of claim 11, wherein said chromoprotein is recovered at a purity of at about 90% or greater based on total protein.

14. The method of claim 11, wherein the anion exchange chromatography matrix is DEAE sepharose.

15. The method of claim 11, wherein the hydrophobic interaction chromatography matrix is phenyl sepharose.

16.-20. (canceled)

21. The method of claim 11, wherein the size exclusion chromatography matrix has a separation range from about 3×10³ to about 7×10⁵.
